(19) United States
(12) Patent Application Publication
(10) Pub. No.: US 2023/0114304 A1
(43) Pub. Date: Apr. 13, 2023

(54) DEPALMITOYLATING COMPOSITIONS AND THE USE THEREOF

(71) Applicant: THE REGENTS OF THE UNIVERSITY OF CALIFORNIA, Oakland, CA (US)

(72) Inventors: Neal Devaraj, La Jolla, CA (US); Andrew Rudd, La Jolla, CA (US); Roberto Javier Brea Fernandez, San Diego, CA (US)

(21) Appl. No.: 17/634,645

(22) PCT Filed: Aug. 14, 2020
(86) PCT No.: PCT/US2020/046411

§ 371 Date: Feb. 11, 2022

Related U.S. Application Data
(60) Provisional application No. 62/887,195, filed on Aug. 15, 2019.

(51) Int. Cl. C07D 207/09; C07D 487/04; C07D 233/64; C07D 311/80; C07D 277/04; C07C 309/54

(52) U.S. Cl. CPC C07D 207/09; C07D 487/04; C07D 233/64; C07D 311/80; C07D 277/04; C07C 309/54

(57) ABSTRACT

Disclosed herein, inter alia, are depalmitoylating compounds, compositions, and methods of use thereof.

### CROSS-REFERENCES TO RELATED APPLICATIONS

This application claims the benefit of U.S. Provisional Application No. 62/887,195, filed Aug. 15, 2019, which is incorporated herein by reference in its entirety and for all purposes.

### BACKGROUND

Post-translational S-palmitoylation plays a central role in protein localization, trafficking, stability, aggregation, and cell signaling. Dysregulation of palmitoylation pathways in cells can alter protein function and is the cause of several diseases. Considering the biological and clinical importance of S-palmitoylation, tools for direct, in vivo modulation of this lipid modification would be extremely valuable. Disclosed herein, inter alia, are solutions to these and other problems in the art.

### BRIEF SUMMARY

In an aspect is provided a compound, or a pharmaceutically acceptable salt thereof, having the formula:

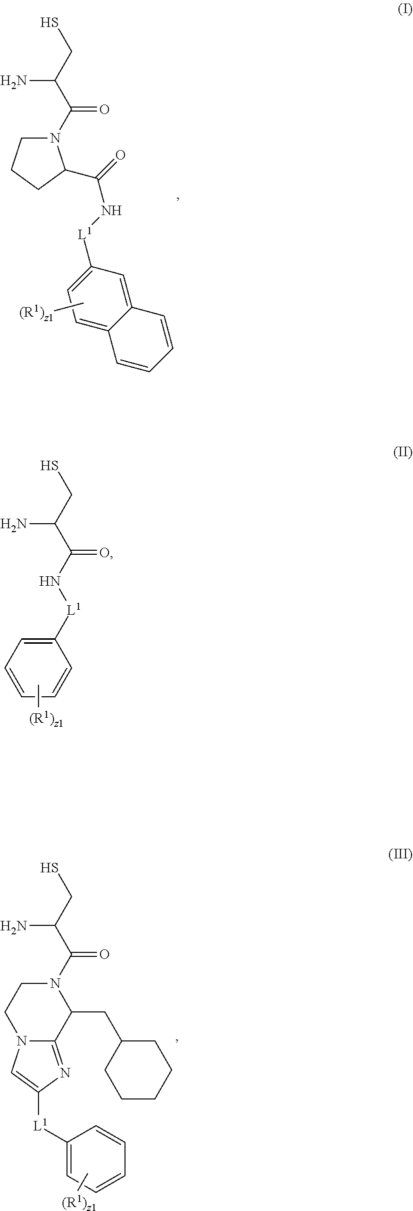

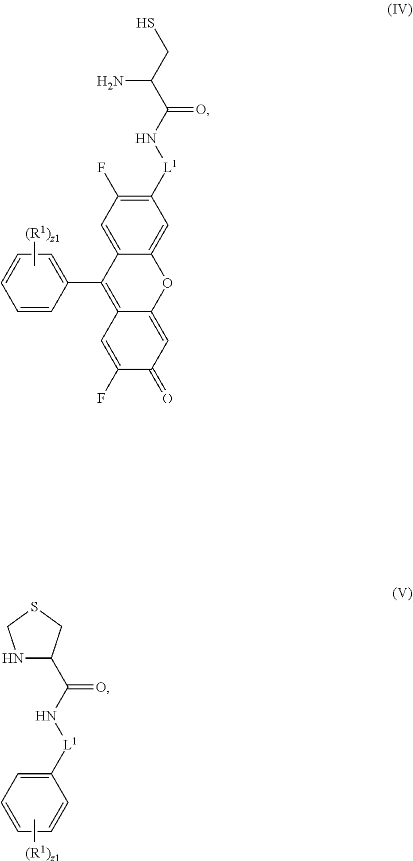

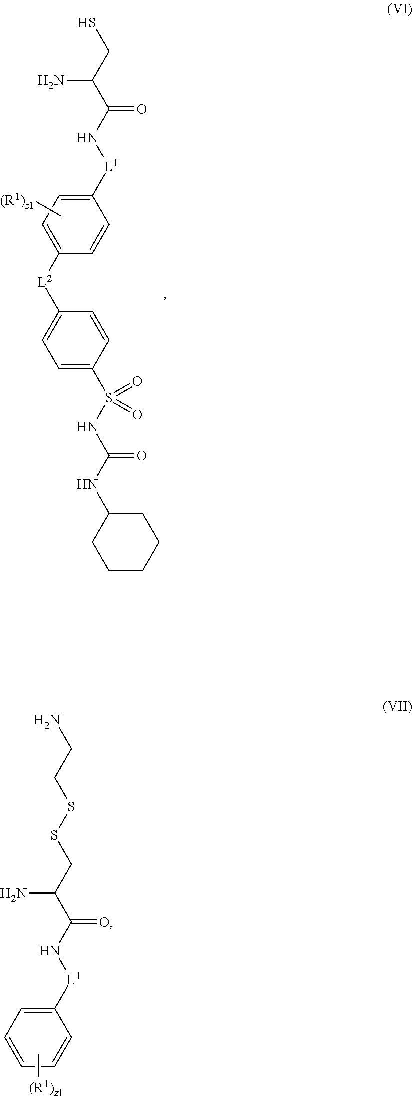

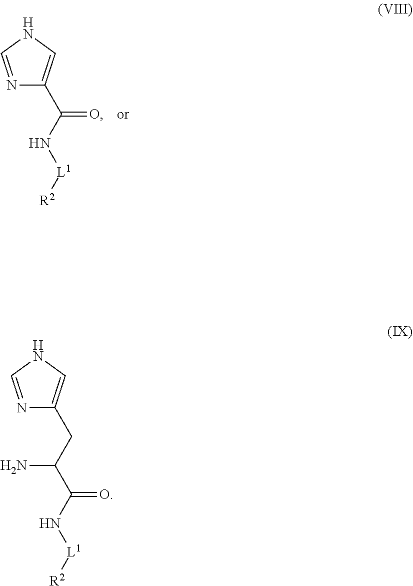

L¹ is a bond, substituted or unsubstituted C₁-C₁₀ alkylene, or substituted or unsubstituted 2 to 10 membered heteroalkylene. L² is a bond, substituted or unsubstituted C₁-C₁₀ alkylene, or substituted or unsubstituted 2 to 10 membered heteroalkylene. R¹ is independently a halogen, substituted or unsubstituted C₁-C₂₀ alkyl, or substituted or unsubstituted 2 to 20 membered heteroalkyl. R² is hydrogen, halogen, substituted or unsubstituted C₁-C₂₀ alkyl, or substituted or unsubstituted 2 to 20 membered heteroalkyl. The variable z1 is an integer from 0 to 7.

In an aspect is provided a compound, or salt thereof, having the formula:

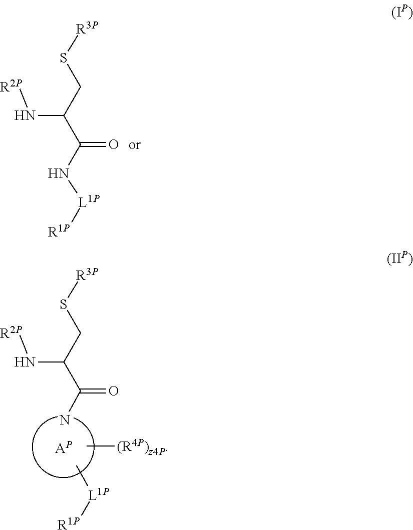

Ring A^{P} is a heterocycloalkyl or heteroaryl.

L^{1P} is L^{101P}-L^{102P}-L^{103P}.

L^{101P} is a bond, —S(O)₂—, —N(R^{101P})—, —O—, —S—, —C(O)—, —C(O)N(R^{101P})—, —N(R^{101P})C(O)—, —N(R^{101P})C(O)NH—, —NHC(O)N(R^{101P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{102P} is a bond, —S(O)₂—, —N(R^{102P})—, —O—, —S—, —C(O)—, —C(O)N(R^{102P})—, —N(R^{102P})C(O)—, —N(R^{102P})C(O)NH—, —NHC(O)N(R^{102P})—, —C(O)—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{03P} is a bond, —S(O)₂—, —N(R^{103P})—, —O—, —S—, —C(O)—, —C(O)N(R^{103P})—, —N(R^{103P})C(O)—, —N(R^{103P})C(O)NH—, —NHC(O)N(R^{103P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R^{101P}, R^{102P}, and R^{103P} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl.

R^{1P} is hydrogen, halogen, —CX^{1P}₃, —CHX^{1P}₂, —CH₂X^{1P}, —OCX^{1P}₃, —OCH₂X^{1P}, —OCHX^{1P}₂, —CN, —SO_{n1P}R^{1DP}, —SO_{vIP}NR^{1AP}R^{1BP}, —NHC(O)NR^{1AP}R^{1BP}, —N(O)_{m1P}, —NR^{1AP}R^{1BP}, —C(O)R^{1CP}, —C(O)OR^{1CP}, —C(O)NR^{1AP}R^{1BP}, —OR^{1DP}, —NR^{1AP}SO₂R^{1DP}, —NR^{1AP}C(O)R^{1CP}, —NR^{1AP}C(O)OR^{1CP}, —NR^{1AP}OR^{1CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2P} is hydrogen, halogen, —CX^{2P}₃, —CHX^{2P}₂, —CH₂X^{2P}, —OCX^{2P}₃, —OCH₂X^{2P}, —OCHX^{2P}₂, —CN, —SO_{n2P}R^{2DP}, —SO_{v2P}NR^{2AP}R^{2BP}, —NHC(O)NR^{2AP}R^{2BP}, —N(O)_{m2P}, —NR^{2AP}R^{2BP}, —C(O)R^{2CP}, —C(O)OR^{2CP}, —C(O)NR^{2AP}R^{2BP}, —OR^{2DP}, —NR^{2AP}SO₂R^{2DP}, —NR^{2AP}C(O)R^{2CP}, —NR^{2AP}C(O)OR^{2CP}, —NR^{2AP}OR^{2CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3P} is hydrogen, halogen, —CX^{3P}₃, —CHX^{3P}₂, —CH₂X^{3P}, —OCX^{3P}₃, —OCH₂X^{3P}, —OCHX^{3P}₂, —CN, —SO_{n3P}R^{3DP}, —SO_{v3P}NR^{3AP}R^{3BP}, —NHC(O)NR^{3AP}R^{3BP}, —N(O)_{m3P}, —NR^{3AP}R^{3BP}, —C(O)R^{3CP}, —C(O)OR^{3CP}, —C(O)NR^{3AP}R^{3BP}, —OR^{3DP}, —NR^{3AP}SO₂R^{3DP}, —NR^{3AP}C(O)R^{3CP}, —NR^{3AP}C(O)OR^{3CP}, —NR^{3AP}OR^{3CP}, —N₃, —SR^{3AP}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1AP}, R^{1BP}, R^{1CP}, R^{1DP}, R^{2AP}, R^{2BP}, R^{2CP}, R^{2DP}, R^{3AP}, R^{3BP}, R^{3CP}, and R^{3DP} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1AP} and R^{1BP} substituents bonded to the same nitrogen atom may be joined to form a substituted or unsubstituted heterocycloalkyl or substituted or unsubstituted heteroaryl; R^{2AP} and R^{2BP} substituents bonded to the same nitrogen atom may be joined to form a substituted or unsubstituted heterocycloalkyl or substituted or unsubstituted heteroaryl; R^{3AP} and R^{3BP} substituents bonded to the same nitrogen atom may be joined to form a substituted or unsubstituted heterocycloalkyl or substituted or unsubstituted heteroaryl.

R^{2P} and R^{3P} substituents may be joined to form a substituted or unsubstituted heterocycloalkyl, or substituted or unsubstituted heteroaryl.

R^{4P} is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

n1P, n2P, and n3P are independently an integer from 0 to 4.

m1P, m2P, m3P, v1P, v2P, and v3P are independently 1 or 2.

X^{1P}, X^{2P}, and X^{3P} are independently —F, —Cl, —Br, or —I.

z4P is an integer from 0 to 6.

In an aspect is provided a pharmaceutical composition including a compound described herein and a pharmaceutically acceptable excipient.

In an aspect is provided a method of treating a depalmitoylation-associated disease in a subject in need thereof, the method including administering to the subject an effective amount of a compound described herein.

In an aspect is provided a method of treating a disease, the method including administering to a subject in need thereof an effective amount of a compound described herein.

In another aspect, a method of depalmitoylating a protein in a cell is provided.

### BRIEF DESCRIPTION OF THE DRAWINGS

FIG. 1. Examples of selective depalmitoylating compounds.

FIG. 2. Identified DPALM hits with improved HRas depalmitoylation activity.

FIG. 3. HRas selective DPALM hits.

FIG. 4. General strategy for synthesis of new DPALMs.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. In embodiments, the heteroalkyl is monounsaturated. In embodiments, the heteroalkyl is polyunsaturated. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl”, by themselves or in combination with other terms, mean, unless otherwise stated, cyclic versions of “alkyl” and “heteroalkyl”, respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene”, alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen”, by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

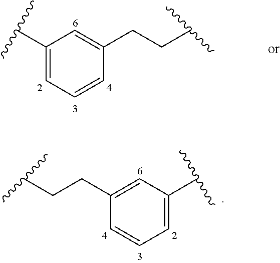 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A3}, R^{4A3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

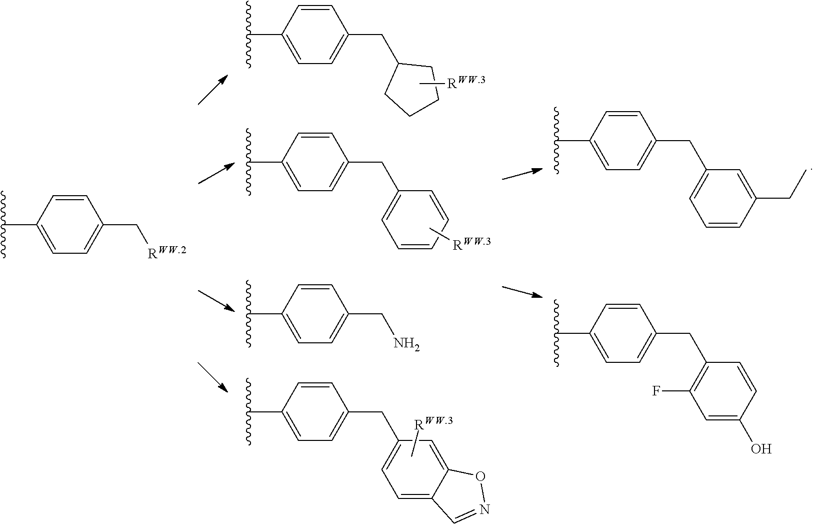

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.2}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The term “bioconjugate reactive moiety” or “bioconjugate reactive group” refers to a chemical moiety which participates in a reaction to form bioconjugate linker (e.g., covalent linker) or the resulting association between atoms or molecules of bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.;
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g., phosphines) to form, for example, phosphate diester bonds;
(n) azides coupled to alkynes using copper catalyzed cycloaddition click chemistry; and
(o) biotin conjugate can react with avidin or strepavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound. A “derivative” is a compound derived from a chemical compound via a chemical reaction. A derivative of a compound described herein may refer to the compound described herein with the addition or removal of a substituent.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. When a substituent or linker (e.g., an R group or an L linker) appears multiple times, each appearance of that substituent or linker can be different, i.e., each occurrence of the substituent or the linker may be independently a member of the Markush group for that variable, wherein each occurrence may be optionally different.

A “detectable agent” or “detectable moiety” is an atom, molecule, substance, or composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Ph, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances. The compositions of the present invention can be delivered transdermally, by a topical route, or formulated as applicator sticks, solutions, suspensions, emulsions, gels, creams, ointments, pastes, jellies, paints, powders, and aerosols.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compounds described herein can be co-administered with conventional neurodegenerative disease treatments including, but not limited to, Parkinson's disease treatments such as levodopa, carbidopa, selegiline, amantadine, donepezil, galanthamine, rivastigmine, tacrine, dopamine agonists (e.g., bromocriptine, pergolide, pramipexole, ropinirole), anticholinergic drugs (e.g., trihexyphenidyl, benztropine, biperiden, procyclidine), and catechol-O-methyl-transferase inhibitors (e.g., tolcapone, entacapone).

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, melphalan), ethylenimine and methylmelamines (e.g., hexamethylmelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

In this disclosure, “comprises,” “comprising,” “containing” and “having” and the like can have the meaning ascribed to them in U.S. patent law and can mean “includes,” “including,” and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “electrophilic” as used herein refers to a chemical group that is capable of accepting electron density. An “electrophilic substituent,” “electrophilic chemical moiety,” or “electrophic moiety” refers to an electron-poor chemical group, substituent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, substituted or unsubstituted piperazinyl, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups are allowed to contact such that the leaving groups are sufficiently proximal to react, interact or physically touch. In embodiments, the leaving groups is designed to facilitate the reaction. In embodiments, the leaving group is a substituent group.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-fluorenylmethyloxycarbonyl (FMOC), acetyl, benzoyl, benzyl, carbamate, p-methoxybenzyl ether (PMB), and tosyl (Ts). In embodiments, the protecting group is —PO₃H or —SO₃H. In embodiments, the protecting group is a substituent group.

The terms “polypeptide,” “peptide,” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g., non-natural or not wild-type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

The term “prevent” refers to a decrease in the occurrence of disease symptoms in a patient. The prevention may be complete (no detectable symptoms) or partial, such that fewer symptoms are observed than would likely occur absent treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition). An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation”, “activate”, “activating”, “activator” and the like in reference to a protein-compound interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor,” “antagonist,” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the composition.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The disease may be a CNS disease. The disease may be a developmental disease. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The term “depalmitoylation-associated disease” is used to broadly refer to disorders or symptoms of diseases associated with a level of depalmitoylation of a protein. In embodiments, the disease is caused by, or a symptom of the disease is caused by, aberrant depalmitoylation (e.g., less or more compared to a standard control). In embodiments, the disease is caused by, or a symptom of the disease is caused by, less depalmitoylation compared to a standard control. In embodiments, the disease is caused by, or a symptom of the disease is caused by, more depalmitoylation compared to a standard control.

The term “associated” or “associated with” in the context of a substance or substance activity (e.g., depalmitoylation activity) or function associated with a disease (e.g., a protein associated disease, a cancer (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity (e.g., depalmitoylation activity) or function. For example, a disease associated with depalmitoylation or a symptom of a depalmitoylation-associated disease or condition associated with an increase or decrease in depalmitoylation activity may be a disease or symptom that results (entirely or partially) from an increase or decrease in depalmitoylation activity (e.g., increase or decrease in depalmitoylation of a protein).

Non-limiting examples of depalmitoylation-associated diseases include cancer and neurodegenerative diseases, e.g., bladder cancer, head and neck cancer, Costello's Syndrome, melanoma, acute myeloid lymphoma (AML), non-small cell lung carcinoma, Alzheimer's disease, infantile neuronal ceroid lipofuscinosis, or glioma.

A “standard control” as referred to herein refers to a sample that serves as a reference, usually a known reference, for comparison to a test sample. For example, a test sample can be taken from a patient suspected of having a depalmitoylation-associated disease (e.g., cancer) and compared to samples from a known depalmitoylation-associated disease (e.g., cancer) patient, or a known normal (non-disease) individual. A control can also represent an average value gathered from a population of similar individuals, e.g., depalmitoylation-associated disease (e.g., cancer) patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One having ordinary skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In some examples of the disclosed methods, when the level of depalmitoylation of a protein (e.g., HRas) is assessed, the level is compared with a control level of depalmitoylation of the same or a different protein. By control level is meant the level of depalmitoylation from a sample or subject lacking a depalmitoylation-associated disease (e.g., cancer), a sample or subject at a selected stage of a depalmitoylation-associated disease (e.g., cancer), or in the absence of a particular variable such as a therapeutic agent (e.g., chemotherapeutic agent). Alternatively, the control level comprises a known amount of depalmitoylation of the protein. Such a known amount correlates with an average level of subjects lacking the depalmitoylation-associated disease (e.g., cancer), at a selected stage of the depalmitoylation-associated disease (e.g., cancer), or in the absence of a particular variable such as a therapeutic agent. A control level also includes the level of depalmitoylation of a protein from one or more selected samples or subjects as described herein. For example, a control level includes an assessment of the level of depalmitoylation of a protein in a sample from a subject that does not have a depalmitoylation-associated disease (e.g., cancer), is not at a selected stage of a depalmitoylation-associated disease (e.g., cancer), or has not received treatment for a depalmitoylation-associated disease (e.g., cancer). Another exemplary control level includes an assessment of the level of depalmitoylation of a protein in samples taken from multiple subjects that do not have a depalmitoylation-associated disease (e.g., cancer), are at a selected stage of a depalmitoylation-associated disease (e.g., cancer), or have not received treatment for a depalmitoylation-associated disease (e.g., cancer).

When the control level includes the level of depalmitoylation of a protein in a sample or subject in the absence of a chemotherapeutic agent, the control sample or subject is optionally the same sample or subject to be tested before or after treatment with a chemotherapeutic agent or is a selected sample or subject in the absence of the therapeutic agent. Alternatively, a control level is an average level calculated from a number of subjects without a particular disease. A control level also includes a known control level or value known in the art.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “central nervous system disease” or “CNS disease” or “neurodegenerative disease” refers to a disease or condition in which the function of a subject's nervous system becomes impaired. Examples of CNS diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “developmental disease” refers to a disease including psychiatric conditions that involve impairment in different areas. Examples of developmental diseases include developmental language disorder, learning disorder, motor disorder, and autism spectrum disorder.

As used herein, the term “inflammatory disease” refers to a disease or condition characterized by aberrant inflammation (e.g., an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The term “infection” or “infectious disease” refers to a disease or condition that can be caused by organisms such as a bacterium, virus, fungi, or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the infectious disease is a viral disease. In embodiments, the infectious disease is a coronavirus infection.

The term “coronavirus” is used in accordance with its plain ordinary meaning and refers to an RNA virus that in humans causes respiratory tract infections. Coronaviruses constitute the subfamily Orthocoronavirinae, in the family Coronaviridae, order Nidovirales, and realm Riboviria. In embodiments, the coronavirus is an enveloped viruses with a positive-sense single-stranded RNA genome.

The term “HRas” or “HRas protein” as used herein refers to any of the recombinant or naturally-occurring forms of the GTPase HRas, also known as transforming protein p21, or variants or homologs thereof that maintain HRas activity (e.g., within at least 50%, 80%, 90%, 95%, 96%, 97%, 98%, 99%, or 100% activity compared to HRas). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring HRas protein. In embodiments, the HRas protein is substantially identical to the protein identified by the NCBI reference number GI: 4885425, or a variant or homolog having substantial identity thereto. In embodiments, the HRas protein is substantially identical to the protein identified by the NCBI reference number GI: 34222246, or a variant or homolog having substantial identity thereto. In embodiments, the HRas protein is substantially identical to the protein identified by the NCBI reference number GI: 194363762, or a variant or homolog having substantial identity thereto. In embodiments, the HRas protein is substantially identical to the protein identified by the NCBI reference number GI: 968121903, or a variant or homolog having substantial identity thereto.

The term “NRas” or “NRas protein” as used herein refers to any of the recombinant or naturally-occurring forms of the GTPase NRas, or variants or homologs thereof that maintain NRas activity (e.g., within at least 50%, 80%, 90%, 95%, 96%, 97%, 98%, 99%, or 100% activity compared to NRas). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring NRas protein. In embodiments, the NRas protein is substantially identical to the protein identified by the NCBI reference number GI: 4505451, or a variant or homolog having substantial identity thereto.

The term “EGFR” or “EGFR protein” as used herein refers to any of the recombinant or naturally-occurring forms of the Epidermal Growth Factor Receptor (EGFR) tyrosine kinase, also known as epidermal growth factor receptor, ErbB-1 or HER1 in humans, or variants or homologs thereof that maintain EGFR activity (e.g., within at least 50%, 80%, 90%, 95%, 96%, 97%, 98%, 99%, or 100% activity compared to EGFR). In some aspects, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring EGFR protein. In embodiments, the EGFR protein is substantially identical to the protein identified by the NCBI reference number GI: 1101020101, or a variant or homolog having substantial identity thereto. In embodiments, the EGFR protein is substantially identical to the protein identified by the NCBI reference number GI: 1100832916, or a variant or homolog having substantial identity thereto. In embodiments, the EGFR protein is substantially identical to the protein identified by the NCBI reference number GI: 1100818978, or a variant or homolog having substantial identity thereto. In embodiments, the EGFR protein is substantially identical to the protein identified by the NCBI reference number GI: 1100818972, or a variant or homolog having substantial identity thereto.

The term “amyloid precursor protein” or “APP” as used herein refers to any of the recombinant or naturally-occurring forms of the amyloid precursor protein, also known as APP, or variants or homologs thereof that maintain amyloid precursor protein activity (e.g., within at least 50%, 80%, 90%, 95%, 96%, 97%, 98%, 99%, or 100% activity compared to amyloid precursor protein). In embodiments, the variants or homologs have at least 90%, 95%, 96%, 97%, 98%, 99% or 100% amino acid sequence identity across the whole sequence or a portion of the sequence (e.g., a 50, 100, 150, or 200 continuous amino acid portion) compared to a naturally occurring amyloid precursor protein. In embodiments, the amyloid precursor protein is substantially identical to the protein identified by the NCBI reference number GI: 4502167, or a variant or homolog having substantial identity thereto. In embodiments, the amyloid precursor protein is substantially identical to the protein identified by the NCBI reference number GI: 41406055, or a variant or homolog having substantial identity thereto. In embodiments, the amyloid precursor protein is substantially identical to the protein identified by the NCBI reference number GI: 41406057, or a variant or homolog having substantial identity thereto. In embodiments, the amyloid precursor protein is substantially identical to the protein identified by the NCBI reference number GI: 209862833, or a variant or homolog having substantial identity thereto.

The term “BACE1” or “BACE1 protein” as used herein refers to any of the recombinant or naturally-occurring forms of the Beta-secretase 1 (BACE1) aspartic-acid protease, also known as beta-site amyloid precursor protein cleaving enzyme 1, beta-site APP cleaving enzyme 1, membrane-associated aspartic protease 2, memapsin-2, aspartyl protease 2, and ASP2, or variants or homologs thereof that maintain BACE1 activity (e.g., within at least 50%, 80%, 90%, 95%, 96%, 97%, 98%, 99%, or 100% activity compared to BACE1). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring BACE1 protein. In embodiments, the BACE1 protein is substantially identical to the protein identified by the NCBI reference number GI: 6912266, or a variant or homolog having substantial identity thereto. In embodiments, the BACE1 protein is substantially identical to the protein identified by the NCBI reference number GI: 21040364, or a variant or homolog having substantial identity thereto. In embodiments, the BACE1 protein is substantially identical to the protein identified by the NCBI reference number GI: 21040366, or a variant or homolog having substantial identity thereto. In embodiments, the BACE1 protein is substantially identical to the protein identified by the NCBI reference number GI: 21040368, or a variant or homolog having substantial identity thereto.

The term “EZH2” or “EZH2 protein” as used herein refers to any of the recombinant or naturally-occurring forms of the Enhancer of Zeste Homolog 2 (EZH2) histone-lysine N-methyltransferase, or variants or homologs thereof that maintain EZH2 activity (e.g., within at least 50%, 80%, 90%, 95%, 96%, 97%, 98%, 99%, or 100% activity compared to EZH2). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring EZH2 protein. In embodiments, the EZH2 protein is substantially identical to the protein identified by the NCBI reference number GI: 21361095, or a variant or homolog having substantial identity thereto. In embodiments, the EZH2 protein is substantially identical to the protein identified by the NCBI reference number GI: 23510384, or a variant or homolog having substantial identity thereto. In embodiments, the EZH2 protein is substantially identical to the protein identified by the NCBI reference number GI: 322506097, or a variant or homolog having substantial identity thereto. In embodiments, the EZH2 protein is substantially identical to the protein identified by the NCBI reference number GI: 322506099, or a variant or homolog having substantial identity thereto.

The term “PD-L1” or “PD-L1 protein” as used herein refers to any of the recombinant or naturally-occurring forms of Programmed Death Ligand 1 (PD-L1), also known as cluster of differentiation 274 (CD274) or B7 homolog 1 (B7-H1), or variants or homologs thereof that maintain PD-L1 activity (e.g., within at least 50%, 80%, 90%, 95%, 96%, 97%, 98%, 99%, or 100% activity compared to PD-L1). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring PD-L1 protein. In embodiments, the PD-L1 protein is substantially identical to the protein identified by the NCBI reference number GI: 7661534, or a variant or homolog having substantial identity thereto. In embodiments, the PD-L1 protein is substantially identical to the protein identified by the NCBI reference number GI: 390979639, or a variant or homolog having substantial identity thereto. In embodiments, the PD-L1 protein is substantially identical to the protein identified by the NCBI reference number GI: 930425329, or a variant or homolog having substantial identity thereto.

The term “flotillin-1” or “flotillin-1 protein” as used herein refers to any of the recombinant or naturally-occurring forms of flotillin-1, or variants or homologs thereof that maintain flotillin-1 activity (e.g., within at least 50%, 80%, 90%, 95%, 96%, 97%, 98%, 99%, or 100% activity compared to flotillin-1). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring flotillin-1 protein. In embodiments, the flotillin-1 protein is substantially identical to the protein identified by the NCBI reference number GI: 5031699, or a variant or homolog having substantial identity thereto. In embodiments, the flotillin-1 protein is substantially identical to the protein identified by the NCBI reference number GI: 974141105, or a variant or homolog having substantial identity thereto.

The term “flotillin-2” or “flotillin-2 protein” as used herein refers to any of the recombinant or naturally-occurring forms of flotillin-2, or variants or homologs thereof that maintain flotillin-2 activity (e.g., within at least 50%, 80%, 90%, 95%, 96%, 97%, 98%, 99%, or 100% activity compared to flotillin-2). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring flotillin-2 protein. In embodiments, the flotillin-2 protein is substantially identical to the protein identified by the NCBI reference number GI: 94538362, or a variant or homolog having substantial identity thereto.

The term “calnexin” or “calnexin protein” as used herein refers to any of the recombinant or naturally-occurring forms of calnexin, or variants or homologs thereof that maintain calnexin activity (e.g., within at least 50%, 80%, 90%, 95%, 96%, 97%, 98%, 99%, or 100% activity compared to calnexin). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring calnexin protein. In embodiments, the calnexin protein is substantially identical to the protein identified by the NCBI reference number GI: 10716563, or a variant or homolog having substantial identity thereto. In embodiments, the calnexin protein is substantially identical to the protein identified by the NCBI reference number GI: 66933005, or a variant or homolog having substantial identity thereto. In embodiments, the calnexin protein is substantially identical to the protein identified by the NCBI reference number GI: 1395168545, or a variant or homolog having substantial identity thereto. In embodiments, the calnexin protein is substantially identical to the protein identified by the NCBI reference number GI: 1395168466, or a variant or homolog having substantial identity thereto.

The term “Gα(i)” or “Gα(i) protein” as used herein refers to any of the recombinant or naturally-occurring forms of Gᵢ alpha subunit (Gα(i)), also known as Gᵢ/G₀ or Gᵢ protein, or variants or homologs thereof that maintain Gα(i) activity (e.g., within at least 50%, 80%, 90%, 95%, 96%, 97%, 98%, 99%, or 100% activity compared to Gα(i)). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring Gα(i) protein. In embodiments, the Gα(i) protein is substantially identical to the protein identified by the NCBI reference number GI: 33946324, or a variant or homolog having substantial identity thereto. In embodiments, the Gα(i) protein is substantially identical to the protein identified by the NCBI reference number GI: 374081863, or a variant or homolog having substantial identity thereto.

The term “metadherin” or “metadherin protein” as used herein refers to any of the recombinant or naturally-occurring forms of metadherin, also known as protein LYRIC or astrocyte elevated gene-1 protein (AEG-1), or variants or homologs thereof that maintain metadherin activity (e.g., within at least 50%, 80%, 90%, 95%, 96%, 97%, 98%, 99%, or 100% activity compared to metadherin). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring metadherin protein. In embodiments, the metadherin protein is substantially identical to the protein identified by the NCBI reference number GI: 223555917, or a variant or homolog having substantial identity thereto. In embodiments, the metadherin protein is substantially identical to the protein identified by the NCBI reference number GI: 1034661969, or a variant or homolog having substantial identity thereto.

The term “CD44” or “CD44 protein” as used herein refers to any of the recombinant or naturally-occurring forms of Cluster of Differentiation 44 (CD44), also known as HCAM (homing cell adhesion molecule), Pgp-1 (phagocytic glycoprotein-1), Hermes antigen, lymphocyte homing receptor, ECM-III, and HUTCH-1, or variants or homologs thereof that maintain CD44 activity (e.g., within at least 50%, 80%, 90%, 95%, 96%, 97%, 98%, 99%, or 100% activity compared to CD44). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring CD44 protein. In embodiments, the CD44 protein is substantially identical to the protein identified by the NCBI reference number GI: 48255941, or a variant or homolog having substantial identity thereto. In embodiments, the CD44 protein is substantially identical to the protein identified by the NCBI reference number GI: 48255935, or a variant or homolog having substantial identity thereto. In embodiments, the CD44 protein is substantially identical to the protein identified by the NCBI reference number GI: 48255937, or a variant or homolog having substantial identity thereto. In embodiments, the CD44 protein is substantially identical to the protein identified by the NCBI reference number GI: 321400138, or a variant or homolog having substantial identity thereto.

The term “SNAP25” or “SNAP25 protein” as used herein refers to any of the recombinant or naturally-occurring forms of Synaptosomal Nerve-Associated Protein 25 (SNAP25), or variants or homologs thereof that maintain SNAP25 activity (e.g., within at least 50%, 80%, 90%, 95%, 96%, 97%, 98%, 99%, or 100% activity compared to SNAP25). In embodiments, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring SNAP25 protein. In embodiments, the SNAP25 protein is substantially identical to the protein identified by the NCBI reference number GI: 18765735, or a variant or homolog having substantial identity thereto. In embodiments, the SNAP25 protein is substantially identical to the protein identified by the NCBI reference number GI: 18765733, or a variant or homolog having substantial identity thereto. In embodiments, the SNAP25 protein is substantially identical to the protein identified by the NCBI reference number GI: 1018443229, or a variant or homolog having substantial identity thereto. In embodiments, the SNAP25 protein is substantially identical to the protein identified by the NCBI reference number GI: 1018443211, or a variant or homolog having substantial identity thereto.

### II. Compounds

In an aspect is provided a compound, or a pharmaceutically acceptable salt thereof, having the formula:

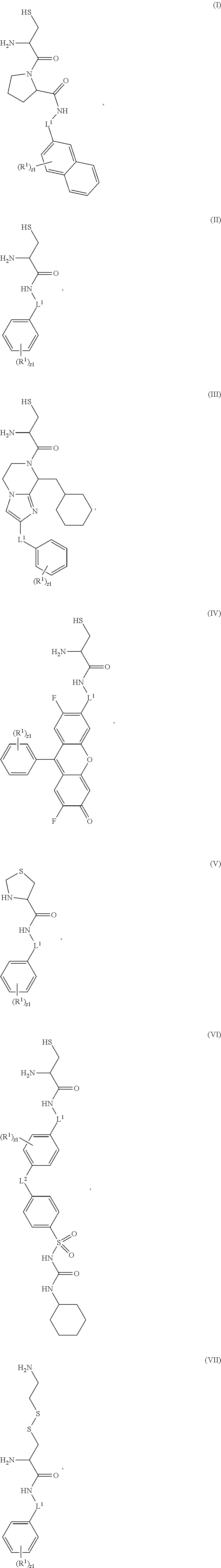

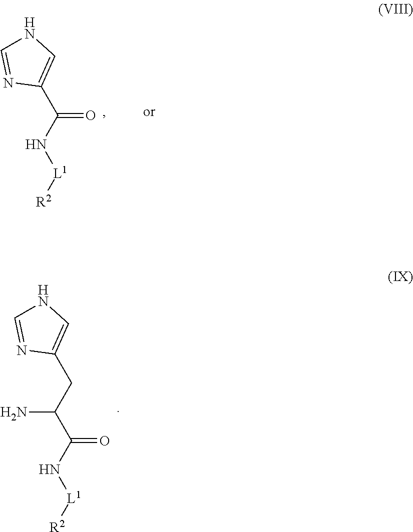

L¹ is a bond, substituted or unsubstituted C₁-C₁₀ alkylene, or substituted or unsubstituted 2 to 10 membered heteroalkylene.

L² is a bond, substituted or unsubstituted C₁-C₁₀ alkylene, or substituted or unsubstituted 2 to 10 membered heteroalkylene.

R¹ is independently a halogen, substituted or unsubstituted C₁-C₂₀ alkyl, or substituted or unsubstituted 2 to 20 membered heteroalkyl.

R² is hydrogen, halogen, substituted or unsubstituted C₁-C₂₀ alkyl, or substituted or unsubstituted 2 to 20 membered heteroalkyl.

The variable z1 is an integer from 0 to 7.

In embodiments, the compound has the formula:

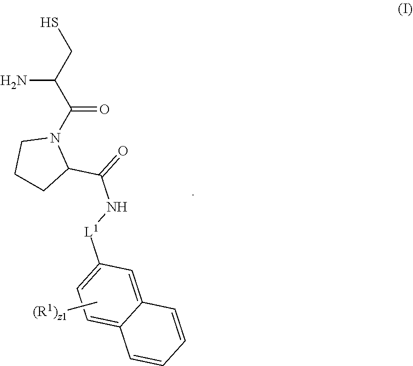

L¹, R¹, and z1 are as described herein, including in embodiments.

In embodiments, the compound has the formula:

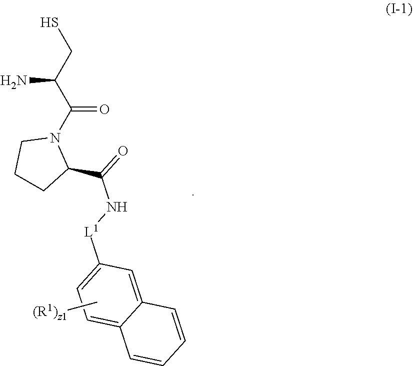

L¹, R¹, and z1 are as described herein, including in embodiments.

In embodiments, the compound has the formula:

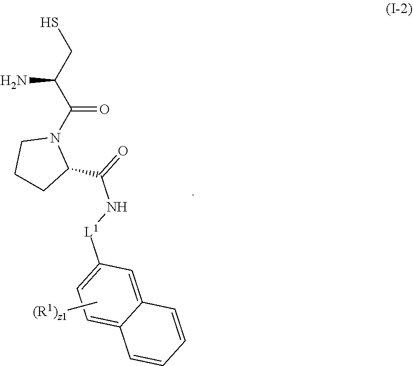

L¹, R¹, and z1 are as described herein, including in embodiments.

In embodiments, the compound has the formula:

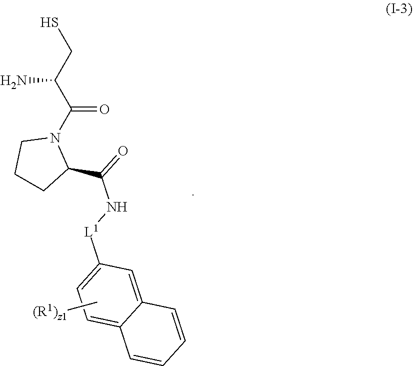

L¹, R¹, and z1 are as described herein, including in embodiments.

In embodiments, the compound has the formula:

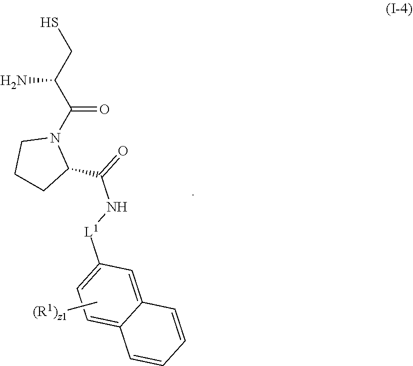

L¹, R¹, and z1 are as described herein, including in embodiments.

In embodiments, the compound has the formula:

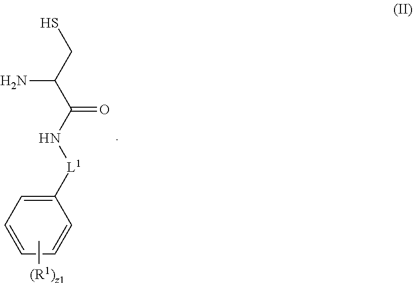

L¹, R¹, and z1 are as described herein, including in embodiments.

In embodiments, the compound has the formula:

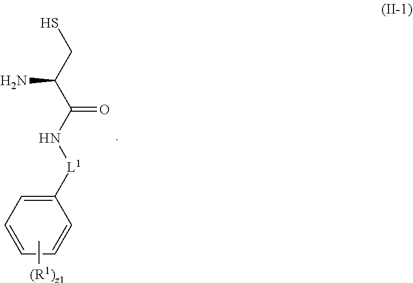

L¹, R¹, and z1 are as described herein, including in embodiments.

In embodiments, the compound has the formula:

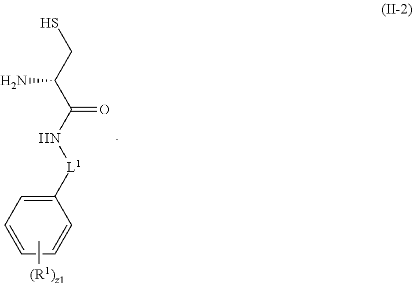

L¹, R¹, and z1 are as described herein, including in embodiments.

In embodiments, the compound has the formula:

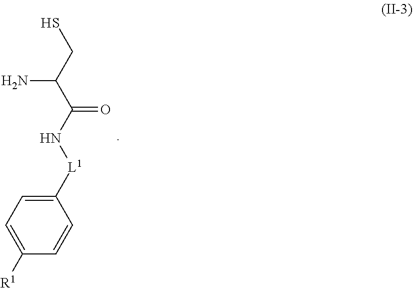

L¹ and R¹ are as described herein, including in embodiments.

In embodiments, the compound has the formula:

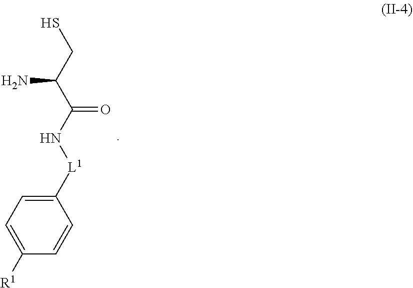

L¹ and R¹ are as described herein, including in embodiments.

In embodiments, the compound has the formula:

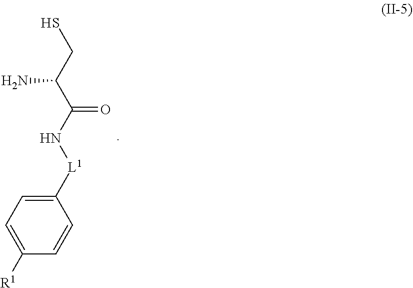

L¹ and R¹ are as described herein, including in embodiments.

In embodiments, the compound has the formula:

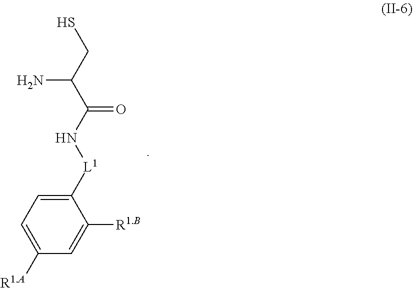

L¹ is as described herein, including in embodiments. R^{1.A} and R^{1.B} may each independently be hydrogen or any value of R¹ as described herein, including in embodiments.

In embodiments, the compound has the formula:

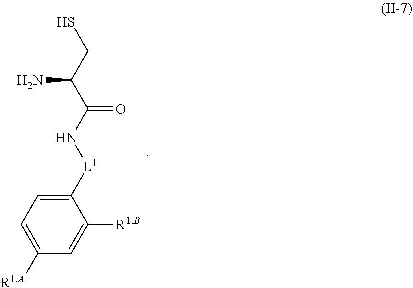

L¹ is as described herein, including in embodiments. R^{1.A} and R^{1.B} may each independently be hydrogen or any value of R¹ as described herein, including in embodiments.

In embodiments, the compound has the formula:

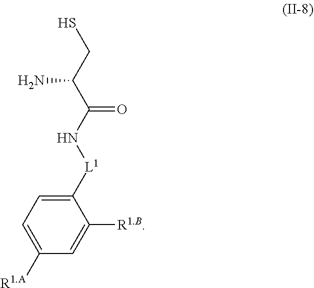

L¹ is as described herein, including in embodiments. R^{1.A} and R^{1.B} may each independently be hydrogen or any value of R¹ as described herein, including in embodiments.

In embodiments, the compound has the formula:

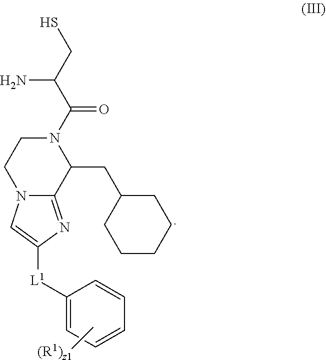

L¹, R¹, and z1 are as described herein, including in embodiments.

In embodiments, the compound has the formula:

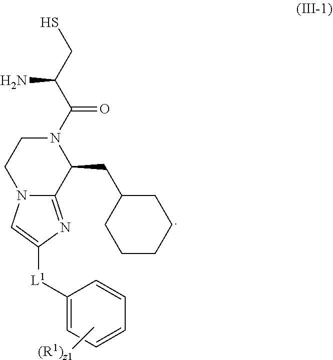

L¹, R¹, and z1 are as described herein, including in embodiments.

In embodiments, the compound has the formula:

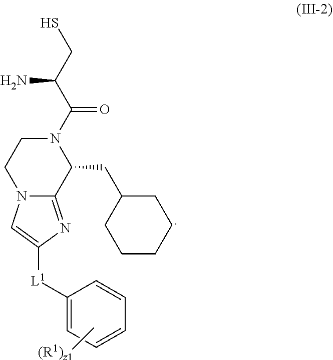

L¹, R¹, and z1 are as described herein, including in embodiments.

In embodiments, the compound has the formula:

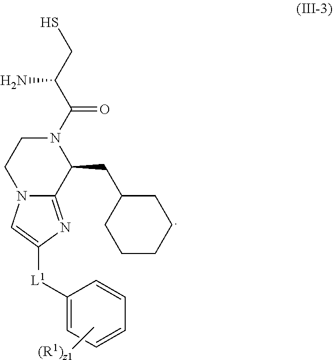

L¹, R¹, and z1 are as described herein, including in embodiments.

In embodiments, the compound has the formula:

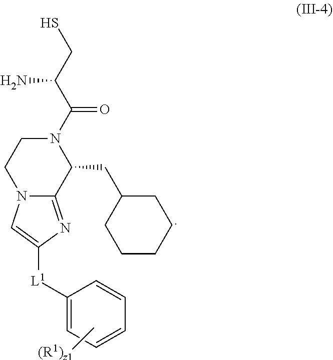

L¹, R¹, and z1 are as described herein, including in embodiments.

In embodiments, the compound has the formula:

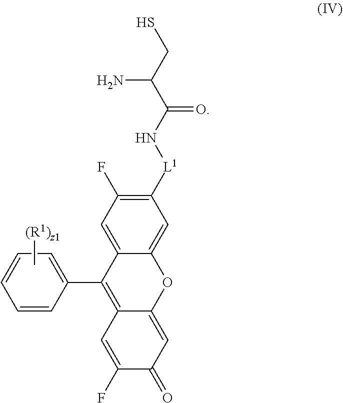

L¹, R¹, and z1 are as described herein, including in embodiments.

In embodiments, the compound has the formula:

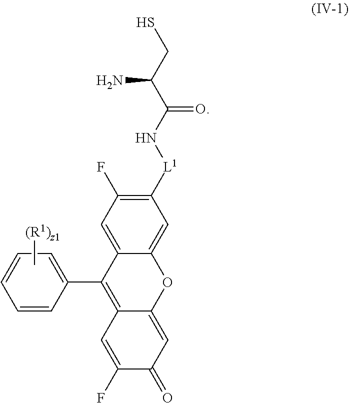

L¹, R¹, and z1 are as described herein, including in embodiments.

In embodiments, the compound has the formula:

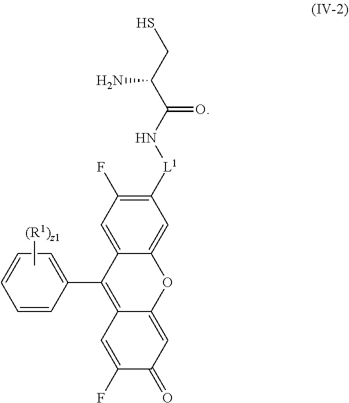

L¹, R¹, and z1 are as described herein, including in embodiments.

In embodiments, the compound has the formula:

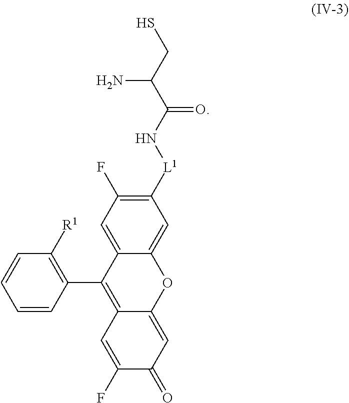

L¹ and R¹ are as described herein, including in embodiments.

In embodiments, the compound has the formula:

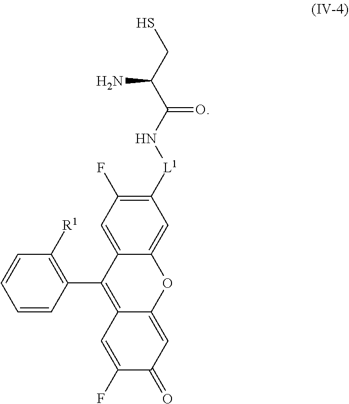

L¹ and R¹ are as described herein, including in embodiments.

In embodiments, the compound has the formula:

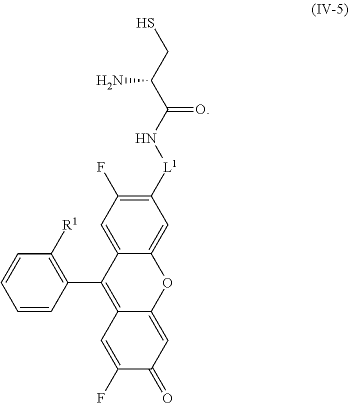

L¹ and R¹ are as described herein, including in embodiments.

In embodiments, the compound has the formula:

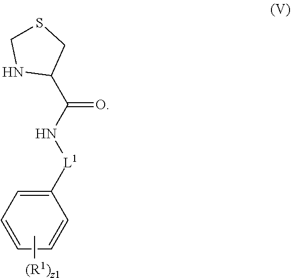

L¹, R¹, and z1 are as described herein, including in embodiments.

In embodiments, the compound has the formula:

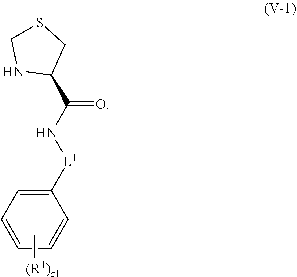

L¹, R¹, and z1 are as described herein, including in embodiments.

In embodiments, the compound has the formula:

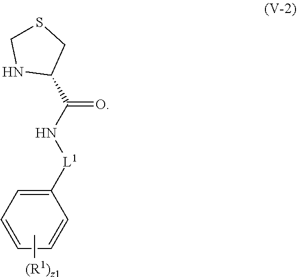

L¹, R¹, and z1 are as described herein, including in embodiments.

In embodiments, the compound has the formula:

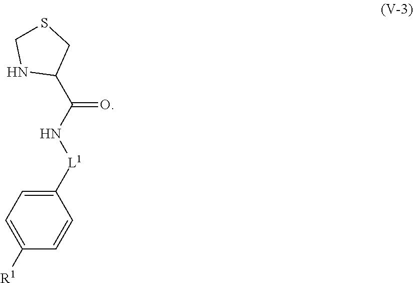

L¹ and R¹ are as described herein, including in embodiments.

In embodiments, the compound has the formula:

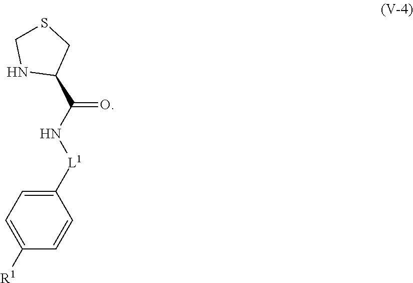

L¹ and R¹ are as described herein, including in embodiments.

In embodiments, the compound has the formula:

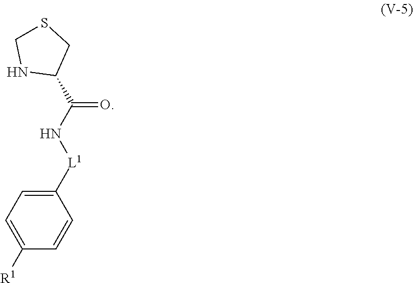

L¹ and R¹ are as described herein, including in embodiments.

In embodiments, the compound has the formula:

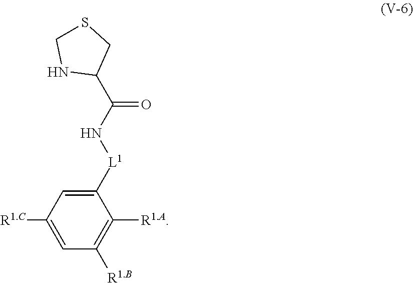

L¹ is as described herein, including in embodiments. R^{1.A}, R^{1.B}, and R^{1.C} may each independently be hydrogen or any value of R¹ as described herein, including in embodiments.

In embodiments, the compound has the formula:

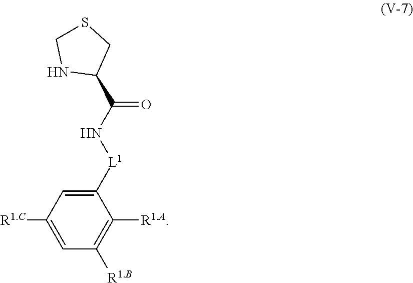

L¹ is as described herein, including in embodiments. R^{1.A}, R^{1.B}, and R^{1.C} may each independently be hydrogen or any value of R¹ as described herein, including in embodiments.

In embodiments, the compound has the formula:

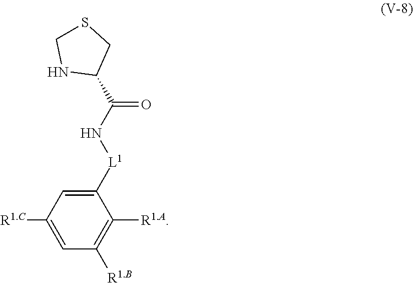

L¹ is as described herein, including in embodiments. R^{1.A}, R^{1.B}, and R^{1.C} may each independently be hydrogen or any value of R¹ as described herein, including in embodiments.

In embodiments, the compound has the formula:

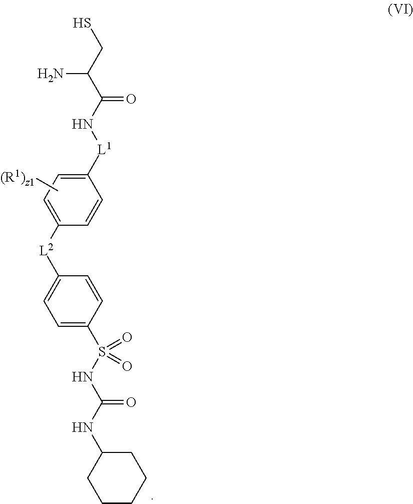

L¹, L², R¹, and z1 are as described herein, including in embodiments.

In embodiments, the compound has the formula:

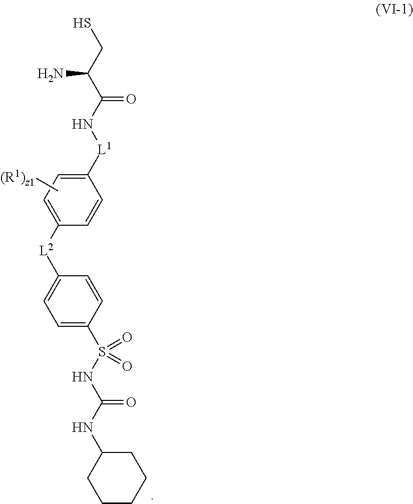

L¹, L², R¹, and z1 are as described herein, including in embodiments.

In embodiments, the compound has the formula:

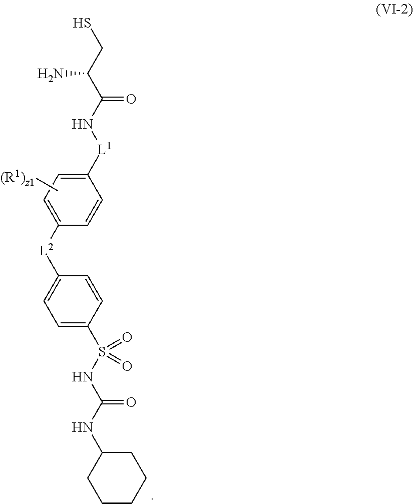

L¹, L², R¹, and z1 are as described herein, including in embodiments.

In embodiments, the compound has the formula:

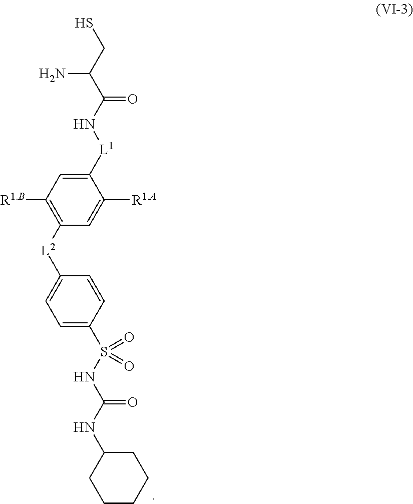

L¹ and L² are as described herein, including in embodiments. R^{1.A} and R^{1.B} may each independently be hydrogen or any value of R¹ as described herein, including in embodiments.

In embodiments, the compound has the formula:

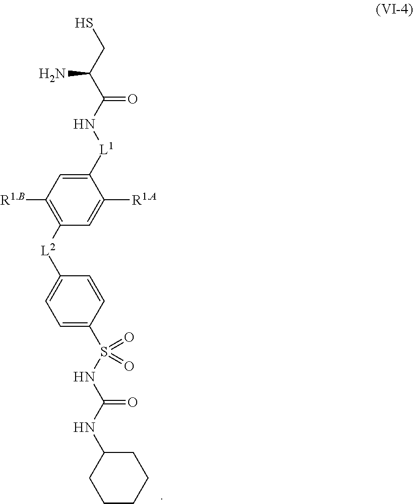

L¹ and L² are as described herein, including in embodiments. R^{1.A} and R^{1.B} may each independently be hydrogen or any value of R¹ as described herein, including in embodiments.

In embodiments, the compound has the formula:

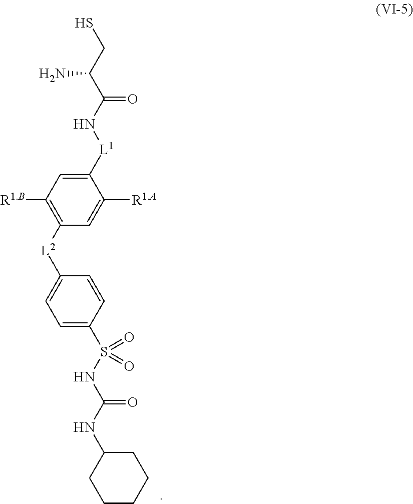

L¹ and L² are as described herein, including in embodiments. R^{1.A} and R^{1.B} may each independently be hydrogen or any value of R¹ as described herein, including in embodiments.

In embodiments, the compound has the formula:

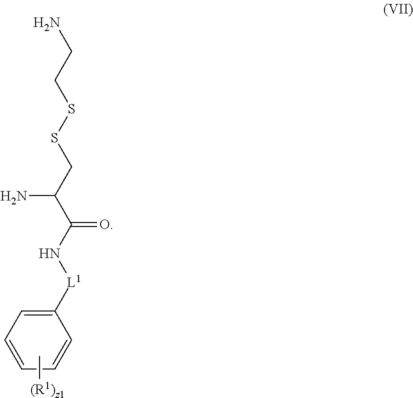

L¹, R¹, and z1 are as described herein, including in embodiments.

In embodiments, the compound has the formula:

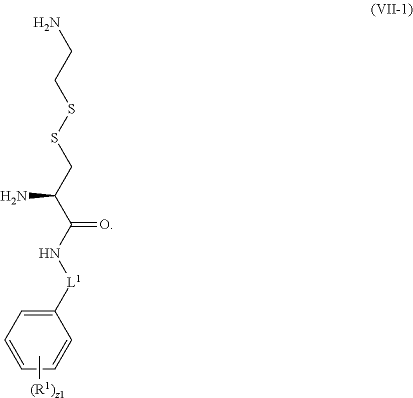

L¹, R¹, and z1 are as described herein, including in embodiments.

In embodiments, the compound has the formula:

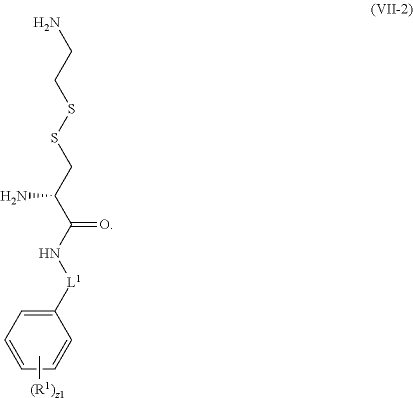

L¹, R¹, and z1 are as described herein, including in embodiments.

In embodiments, the compound has the formula:

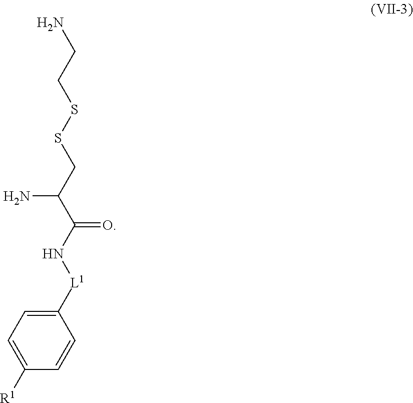

L¹ and R¹ are as described herein, including in embodiments.

In embodiments, the compound has the formula:

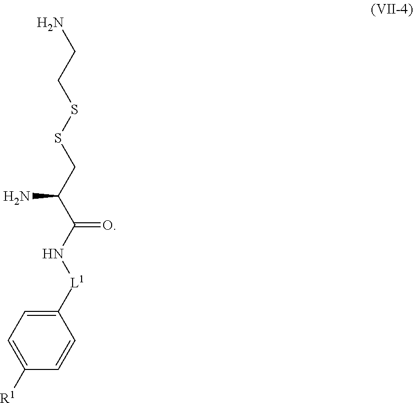

L¹ and R¹ are as described herein, including in embodiments.

In embodiments, the compound has the formula:

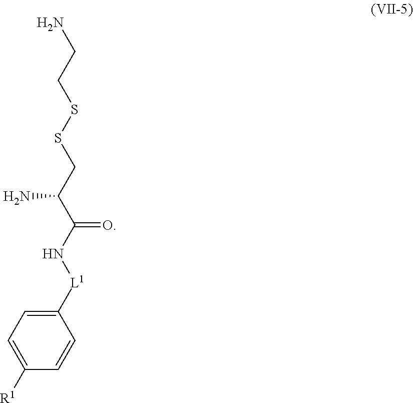

L¹ and R¹ are as described herein, including in embodiments.

In embodiments, the compound has the formula:

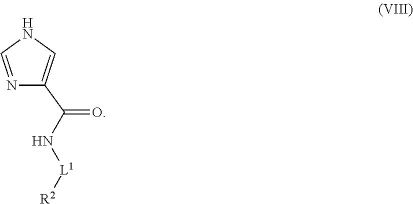

L¹ and R² are as described herein, including in embodiments.

In embodiments, the compound has the formula:

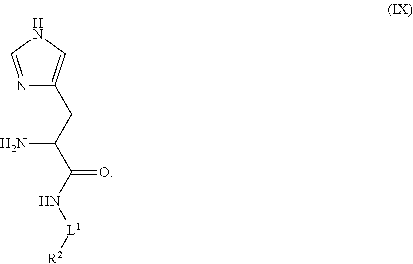

L¹ and R² are as described herein, including in embodiments.

In embodiments, the compound has the formula:

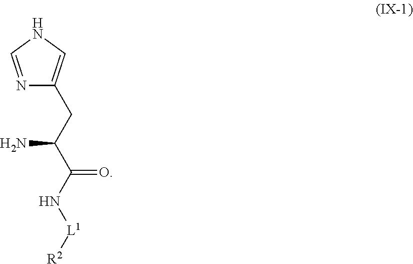

L¹ and R² are as described herein, including in embodiments.

In embodiments, the compound has the formula:

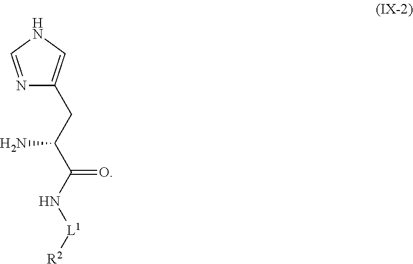

L¹ and R² are as described herein, including in embodiments.

In embodiments, a substituted L¹ (e.g., substituted alkylene and/or substituted heteroalkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, substituted or unsubstituted C₁-C₁₀ alkylene, or substituted or unsubstituted 2 to 10 membered heteroalkylene. In embodiments, L¹ is a bond. In embodiments, L¹ is substituted or unsubstituted C₁-C₁₀ alkylene. In embodiments, L¹ is substituted C₁-C₁₀ alkylene. In embodiments, L¹ is substituted methylene. In embodiments, L¹ is substituted ethylene. In embodiments, L¹ is substituted propylene. In embodiments, L¹ is substituted n-propylene. In embodiments, L¹ is substituted butylene. In embodiments, L¹ is substituted n-butylene. In embodiments, L¹ is substituted pentylene. In embodiments, L¹ is substituted n-pentylene. In embodiments, L¹ is substituted hexylene. In embodiments, L¹ is substituted n-hexylene. In embodiments, L¹ is substituted heptylene. In embodiments, L¹ is substituted n-heptylene. In embodiments, L¹ is substituted octylene. In embodiments, L¹ is substituted n-octylene. In embodiments, L¹ is substituted nonylene. In embodiments, L¹ is substituted n-nonylene. In embodiments, L¹ is substituted decylene. In embodiments, L¹ is substituted n-decylene. In embodiments, L¹ is oxo-substituted C₁-C₁₀ alkylene. In embodiments, L¹ is oxo-substituted methylene. In embodiments, L¹ is oxo-substituted ethylene. In embodiments, L¹ is oxo-substituted propylene. In embodiments, L¹ is oxo-substituted n-propylene. In embodiments, L¹ is oxo-substituted butylene. In embodiments, L¹ is oxo-substituted n-butylene. In embodiments, L¹ is oxo-substituted pentylene. In embodiments, L¹ is oxo-substituted n-pentylene. In embodiments, L¹ is oxo-substituted hexylene. In embodiments, L¹ is oxo-substituted n-hexylene. In embodiments, L¹ is oxo-substituted heptylene. In embodiments, L¹ is oxo-substituted n-heptylene. In embodiments, L¹ is oxo-substituted octylene. In embodiments, L¹ is oxo-substituted n-octylene. In embodiments, L¹ is oxo-substituted nonylene. In embodiments, L¹ is oxo-substituted n-nonylene. In embodiments, L¹ is oxo-substituted decylene. In embodiments, L¹ is oxo-substituted n-decylene. In embodiments, L¹ is substituted 2 to 10 membered heteroalkylene. In embodiments, L¹ is -(substituted C₁-C₆ alkylene)-NH—. In embodiments, L¹ is oxo-substituted 2 to 10 membered heteroalkylene. In embodiments, L¹ is -(oxo-substituted C₁-C₆ alkylene)-NH—.

In embodiments, L¹ is a bond, unsubstituted C₁-C₁₀ alkylene, or unsubstituted 2 to 10 membered heteroalkylene. In embodiments, L¹ is a bond. In embodiments, L¹ is unsubstituted C₁-C₁₀ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted tert-butylene. In embodiments, L¹ is unsubstituted pentylene. In embodiments, L¹ is unsubstituted n-pentylene. In embodiments, L¹ is unsubstituted hexylene. In embodiments, L¹ is unsubstituted n-hexylene. In embodiments, L¹ is unsubstituted heptylene. In embodiments, L¹ is unsubstituted n-heptylene. In embodiments, L¹ is unsubstituted octylene. In embodiments, L¹ is unsubstituted n-octylene. In embodiments, L¹ is unsubstituted nonylene. In embodiments, L¹ is unsubstituted n-nonylene. In embodiments, L¹ is unsubstituted decylene. In embodiments, L¹ is unsubstituted n-decylene. In embodiments, L¹ is unsubstituted 2 to 10 membered heteroalkylene. In embodiments, L¹ is -(unsubstituted C₁-C₆ alkylene)-NH—. In embodiments, L¹ is —CH₂—NH—. In embodiments, L¹ is —CH₂—CH₂—NH—. In embodiments, L¹ is —(CH₂)₃—NH—. In embodiments, L¹ is —(CH₂)₄—NH—. In embodiments, L¹ is —(CH₂)₅—NH—. In embodiments, L¹ is —(CH₂)₆—NH—.

In embodiments, a substituted L² (e.g., substituted alkylene and/or substituted heteroalkylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a bond, substituted or unsubstituted C₁-C₁₀ alkylene, or substituted or unsubstituted 2 to 10 membered heteroalkylene. In embodiments, L² is a bond. In embodiments, L² is substituted or unsubstituted C₁-C₁₀ alkylene. In embodiments, L² is substituted C₁-C₁₀ alkylene. In embodiments, L² is substituted methylene. In embodiments, L² is substituted ethylene. In embodiments, L² is substituted propylene. In embodiments, L² is substituted n-propylene. In embodiments, L² is substituted butylene. In embodiments, L² is substituted n-butylene. In embodiments, L² is substituted pentylene. In embodiments, L² is substituted n-pentylene. In embodiments, L² is substituted hexylene. In embodiments, L² is substituted n-hexylene. In embodiments, L² is substituted heptylene. In embodiments, L² is substituted n-heptylene. In embodiments, L² is substituted octylene. In embodiments, L² is substituted n-octylene. In embodiments, L² is substituted nonylene. In embodiments, L² is substituted n-nonylene. In embodiments, L² is substituted decylene. In embodiments, L² is substituted n-decylene. In embodiments, L² is oxo-substituted C₁-C₁₀ alkylene. In embodiments, L² is oxo-substituted methylene. In embodiments, L² is oxo-substituted ethylene. In embodiments, L² is oxo-substituted propylene. In embodiments, L² is oxo-substituted n-propylene. In embodiments, L² is oxo-substituted butylene. In embodiments, L² is oxo-substituted n-butylene. In embodiments, L² is oxo-substituted pentylene. In embodiments, L² is oxo-substituted n-pentylene. In embodiments, L² is oxo-substituted hexylene. In embodiments, L² is oxo-substituted n-hexylene. In embodiments, L² is oxo-substituted heptylene. In embodiments, L² is oxo-substituted n-heptylene. In embodiments, L² is oxo-substituted octylene. In embodiments, L² is oxo-substituted n-octylene. In embodiments, L² is oxo-substituted nonylene. In embodiments, L² is oxo-substituted n-nonylene. In embodiments, L² is oxo-substituted decylene. In embodiments, L² is oxo-substituted n-decylene.

In embodiments, L² is a bond, unsubstituted C₁-C₁₀ alkylene, or unsubstituted 2 to 10 membered heteroalkylene. In embodiments, L² is a bond. In embodiments, L² is unsubstituted C₁-C₁₀ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted isopropylene. In embodiments, L² is unsubstituted butylene. In embodiments, L² is unsubstituted n-butylene. In embodiments, L² is unsubstituted tert-butylene. In embodiments, L² is unsubstituted pentylene. In embodiments, L² is unsubstituted n-pentylene. In embodiments, L² is unsubstituted hexylene. In embodiments, L² is unsubstituted n-hexylene. In embodiments, L² is unsubstituted heptylene. In embodiments, L² is unsubstituted n-heptylene. In embodiments, L² is unsubstituted octylene. In embodiments, L² is unsubstituted n-octylene. In embodiments, L² is unsubstituted nonylene. In embodiments, L² is unsubstituted n-nonylene. In embodiments, L² is unsubstituted decylene. In embodiments, L² is unsubstituted n-decylene. In embodiments, L² is unsubstituted 2 to 10 membered heteroalkylene. In embodiments, L² is -(unsubstituted C₁-C₆ alkylene)-NH—. In embodiments, L² is —CH₂—NH—. In embodiments, L² is —CH₂—CH₂—NH—. In embodiments, L² is —(CH₂)₃—NH—. In embodiments, L² is —(CH₂)₄—NH—. In embodiments, L² is —(CH₂)₅—NH—. In embodiments, L² is —(CH₂)₆—NH—.

In embodiments, L² is a bond or oxo-substituted 2 to 10 membered heteroalkylene. In embodiments, L² is a bond. In embodiments, L² is substituted 2 to 10 membered heteroalkylene. In embodiments, L² is oxo-substituted 2 to 10 membered heteroalkylene. In embodiments, L² is —C(O)NH-(substituted C₁-C₈ alkylene)-. In embodiments, L² is —C(O)NH-(substituted C₁-C₆ alkylene)-. In embodiments, L² is —C(O)NH-(unsubstituted C₁-C₈ alkylene)-. In embodiments, L² is —C(O)NH-(unsubstituted C₁-C₆ alkylene)-. In embodiments, L² is —C(O)NH—CH₂—. In embodiments, L² is —C(O)NH—CH₂—CH₂—. In embodiments, L² is —C(O)NH—(CH₂)₃—. In embodiments, L² is —C(O)NH—(CH₂)₄—. In embodiments, L² is —C(O)NH—(CH₂)₅—. In embodiments, L² is —C(O)NH—(CH₂)₆—. In embodiments, L² is —C(O)NH—(CH₂)₇—. In embodiments, L² is —C(O)NH—(CH₂)₈—.

In embodiments, a substituted R¹ (e.g., substituted alkyl and/or substituted heteroalkyl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is independently a halogen, unsubstituted C₁-C₁₀ alkyl, or substituted or unsubstituted 2 to 10 membered heteroalkyl. In embodiments, R¹ is independently a halogen, unsubstituted C₁-C₆ alkyl, or unsubstituted C₁-C₆ alkoxy. In embodiments, R¹ is independently —F. In embodiments, R¹ is independently —Cl. In embodiments, R¹ is independently —Br. In embodiments, R¹ is independently —I. In embodiments, R¹ is independently an unsubstituted C₁-C₁₀ alkyl. In embodiments, R¹ is independently an unsubstituted C₁-C₆ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted tert-butyl. In embodiments, R¹ is independently unsubstituted pentyl. In embodiments, R¹ is independently unsubstituted n-pentyl. In embodiments, R¹ is independently an unsubstituted hexyl. In embodiments, R¹ is independently an unsubstituted n-hexyl. In embodiments, R¹ is independently an unsubstituted heptyl. In embodiments, R¹ is independently an unsubstituted n-heptyl. In embodiments, R¹ is independently an unsubstituted octyl. In embodiments, R¹ is independently an unsubstituted n-octyl. In embodiments, R¹ is independently an unsubstituted nonyl. In embodiments, R¹ is independently an unsubstituted n-nonyl. In embodiments, R¹ is independently an unsubstituted decyl. In embodiments, R¹ is independently an unsubstituted n-decyl. In embodiments, R¹ is independently an unsubstituted C₁-C₁₀ alkoxy. In embodiments, R¹ is independently an unsubstituted C₁-C₆ alkoxy. In embodiments, R¹ is independently unsubstituted —O-methyl. In embodiments, R¹ is independently unsubstituted —O-ethyl. In embodiments, R¹ is independently unsubstituted —O-propyl. In embodiments, R¹ is independently unsubstituted —O-n-propyl. In embodiments, R¹ is independently unsubstituted —O-isopropyl. In embodiments, R¹ is independently unsubstituted —O-butyl. In embodiments, R¹ is independently unsubstituted —O-n-butyl. In embodiments, R¹ is independently unsubstituted —O-tert-butyl. In embodiments, R¹ is independently unsubstituted —O-pentyl. In embodiments, R¹ is independently unsubstituted —O-n-pentyl. In embodiments, R¹ is independently unsubstituted —O-hexyl. In embodiments, R¹ is independently unsubstituted —O-n-hexyl. In embodiments, R¹ is independently unsubstituted —O-heptyl. In embodiments, R¹ is independently unsubstituted —O-n-heptyl. In embodiments, R¹ is independently unsubstituted —O-octyl. In embodiments, R¹ is independently unsubstituted —O-n-octyl. In embodiments, R¹ is independently unsubstituted —O-nonyl. In embodiments, R¹ is independently unsubstituted —O-n-nonyl. In embodiments, R¹ is independently unsubstituted —O-decyl. In embodiments, R¹ is independently unsubstituted —O-n-decyl.

In embodiments, a substituted R² (e.g., substituted alkyl and/or substituted heteroalkyl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is an unsubstituted C₁-C₂₀ alkyl, or unsubstituted 2 to 20 membered heteroalkyl. In embodiments, R² is hydrogen. In embodiments, R² is an unsubstituted C₁-C₁₀ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted tert-butyl. In embodiments, R² is unsubstituted pentyl. In embodiments, R² is unsubstituted n-pentyl. In embodiments, R² is unsubstituted hexyl. In embodiments, R² is unsubstituted n-hexyl. In embodiments, R² is unsubstituted heptyl. In embodiments, R² is unsubstituted n-heptyl. In embodiments, R² is unsubstituted octyl. In embodiments, R² is unsubstituted n-octyl. In embodiments, R² is unsubstituted nonyl. In embodiments, R² is unsubstituted n-nonyl. In embodiments, R² is unsubstituted decyl. In embodiments, R² is unsubstituted n-decyl. In embodiments, R² is independently an unsubstituted C₁-C₁₀ alkoxy. In embodiments, R² is unsubstituted —O-methyl. In embodiments, R² is unsubstituted —O-ethyl. In embodiments, R² is unsubstituted —O-propyl. In embodiments, R² is unsubstituted —O-n-propyl. In embodiments, R² is unsubstituted —O-isopropyl. In embodiments, R² is unsubstituted —O-butyl. In embodiments, R² is unsubstituted —O-n-butyl. In embodiments, R² is unsubstituted —O-tert-butyl. In embodiments, R² is unsubstituted —O-pentyl. In embodiments, R² is unsubstituted —O-n-pentyl. In embodiments, R² is unsubstituted —O-hexyl. In embodiments, R² is unsubstituted —O-n-hexyl. In embodiments, R² is unsubstituted —O-heptyl. In embodiments, R² is unsubstituted —O-n-heptyl. In embodiments, R² is unsubstituted —O-octyl. In embodiments, R² is unsubstituted —O-n-octyl. In embodiments, R² is unsubstituted —O-nonyl. In embodiments, R² is unsubstituted —O-n-nonyl. In embodiments, R² is unsubstituted —O-decyl. In embodiments, R² is unsubstituted —O-n-decyl.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7.

In embodiments, when z1 is an integer from 2 to 7, each R¹ may optionally be different.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², R^{L2.1}, R^{L2.2}, and R^{L2.3}, respectively.

In embodiments, the compound has the formula:

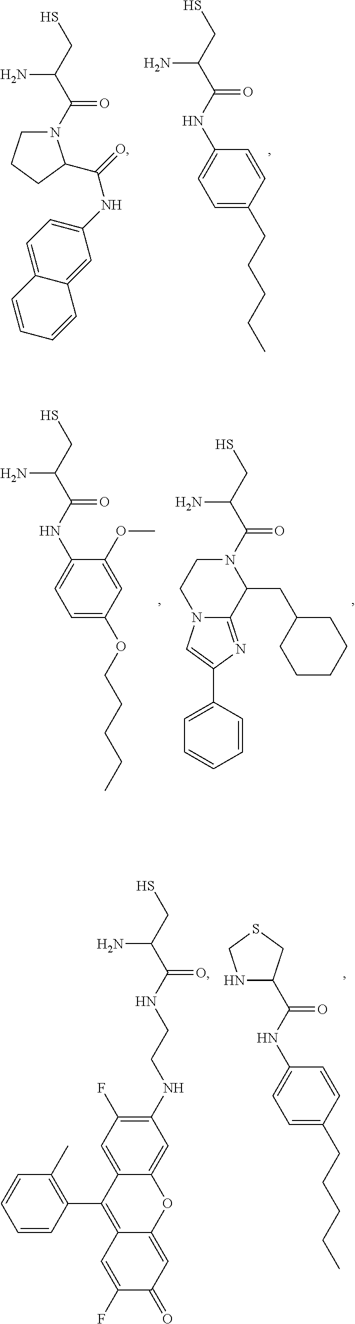

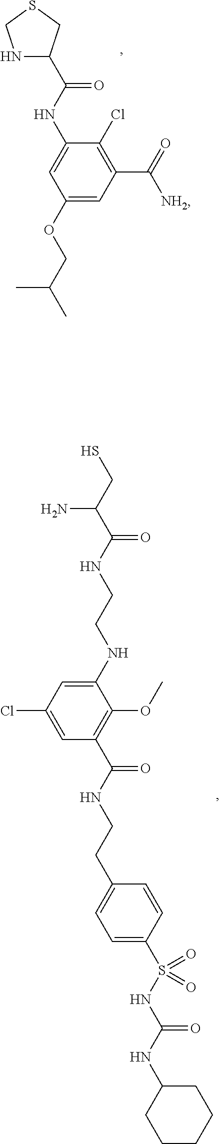 [SMILES: CC(C)COc1cc(NC(=O)C2CSCN2)c(Cl)c(C(N)=O)c1 ; COc1c(NCCNC(=O)C(N)CS)cc(Cl)cc1C(=O)NCCc1ccc(S(=O)(=O)NC(=O)NC2CCCCC2)cc1]

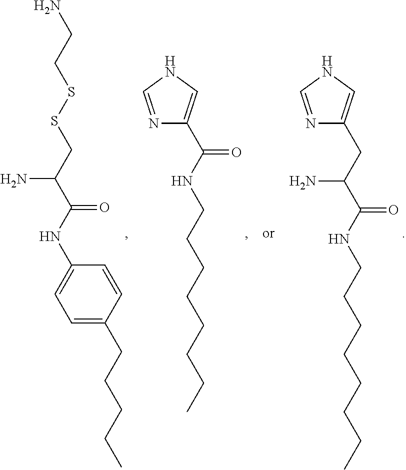 [SMILES: CCCCCc1ccc(NC(=O)C(N)CSSCCN)cc1 ; CCCCCCCCNC(=O)c1c[nH]cn1 ; CCCCCCCCNC(=O)C(N)Cc1c[nH]cn1]

In embodiments, the compound has the formula

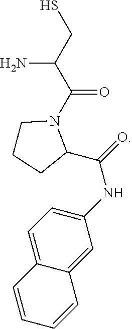 [SMILES: NC(CS)C(=O)N1CCCC1C(=O)Nc1ccc2ccccc2c1]

In embodiments, the compound has the formula

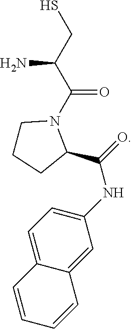 [SMILES: N[C@@H](CS)C(=O)N1CCC[C@@H]1C(=O)Nc1ccc2ccccc2c1]

In embodiments, the compound has the formula

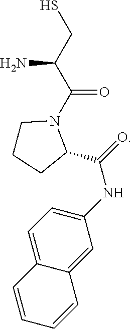 [SMILES: N[C@@H](CS)C(=O)N1CCC[C@H]1C(=O)Nc1ccc2ccccc2c1]

In embodiments, the compound has the formula

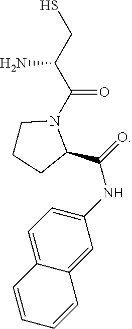 [SMILES: N[C@H](CS)C(=O)N1CCC[C@@H]1C(=O)Nc1ccc2ccccc2c1]

In embodiments, the compound has the formula

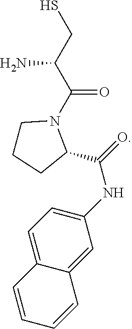 [SMILES: N[C@H](CS)C(=O)N1CCC[C@H]1C(=O)Nc1ccc2ccccc2c1]

In embodiments, the compound has the formula

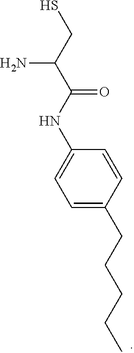 [SMILES: CCCCCc1ccc(NC(=O)C(N)CS)cc1]

In embodiments, the compound has the formula

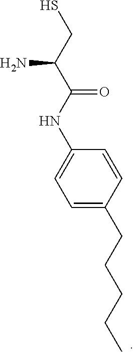 [SMILES: CCCCCc1ccc(NC(=O)[C@@H](N)CS)cc1]

In embodiments, the compound has the formula

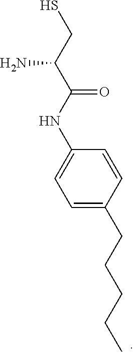 [SMILES: CCCCCc1ccc(NC(=O)[C@H](N)CS)cc1]

In embodiments, the compound has the formula

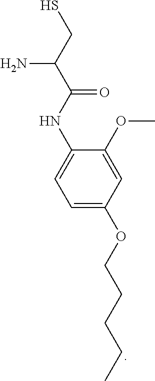 [SMILES: CCCCCOc1ccc(NC(=O)C(N)CS)c(OC)c1]

In embodiments, the compound has the formula

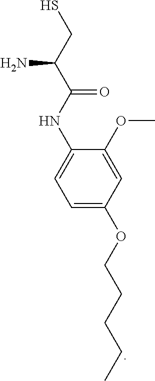 [SMILES: CCCCCOc1ccc(NC(=O)[C@@H](N)CS)c(OC)c1]

In embodiments, the compound has the formula

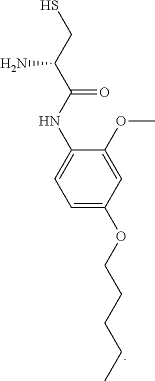 [SMILES: CCCCCOc1ccc(NC(=O)[C@H](N)CS)c(OC)c1]

In embodiments, the compound has the formula

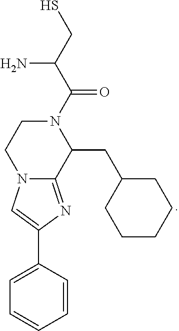 [SMILES: NC(CS)C(=O)N1CCn2cc(-c3ccccc3)nc2C1CC1CCCCC1]

In embodiments, the compound has the formula

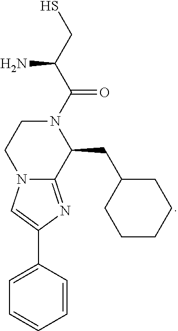 [SMILES: N[C@@H](CS)C(=O)N1CCn2cc(-c3ccccc3)nc2[C@@H]1CC1CCCCC1]

In embodiments, the compound has the formula

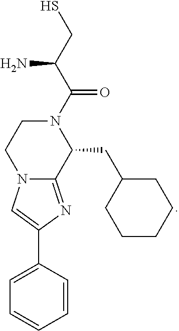 [SMILES: N[C@@H](CS)C(=O)N1CCn2cc(-c3ccccc3)nc2[C@H]1CC1CCCCC1]

In embodiments, the compound has the formula

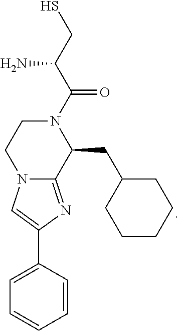 [SMILES: N[C@H](CS)C(=O)N1CCn2cc(-c3ccccc3)nc2[C@@H]1CC1CCCCC1]

In embodiments, the compound has the formula

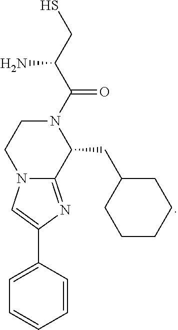 [SMILES: N[C@H](CS)C(=O)N1CCn2cc(-c3ccccc3)nc2[C@H]1CC1CCCCC1]

In embodiments, the compound has the formula

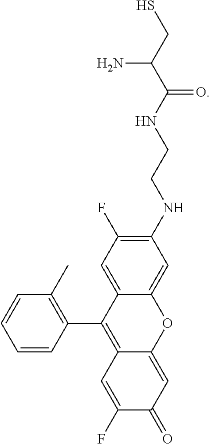 [SMILES: Cc1ccccc1-c1c2cc(F)c(=O)cc-2oc2cc(NCCNC(=O)C(N)CS)c(F)cc12]

In embodiments, the compound has the formula

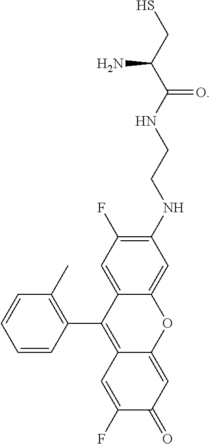 [SMILES: Cc1ccccc1-c1c2cc(F)c(=O)cc-2oc2cc(NCCNC(=O)[C@@H](N)CS)c(F)cc12]

In embodiments, the compound has the formula

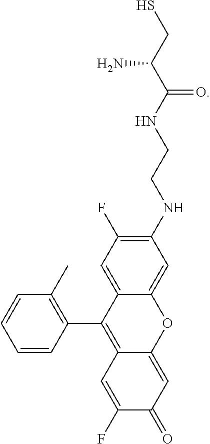 [SMILES: Cc1ccccc1-c1c2cc(F)c(=O)cc-2oc2cc(NCCNC(=O)[C@H](N)CS)c(F)cc12]

In embodiments, the compound has the formula

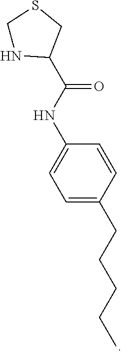 [SMILES: CCCCCc1ccc(NC(=O)C2CSCN2)cc1]

In embodiments, the compound has the formula

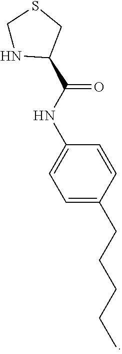 [SMILES: CCCCCc1ccc(NC(=O)[C@@H]2CSCN2)cc1]

In embodiments, the compound has the formula

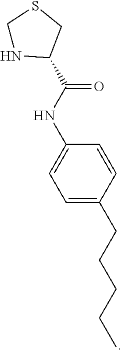 [SMILES: CCCCCc1ccc(NC(=O)[C@H]2CSCN2)cc1]

In embodiments, the compound has the formula

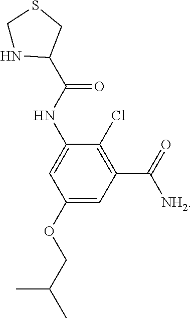 [SMILES: CC(C)COc1cc(NC(=O)C2CSCN2)c(Cl)c(C(N)=O)c1]

In embodiments, the compound has the formula

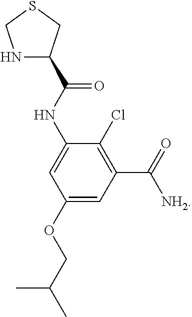 [SMILES: CC(C)COc1cc(NC(=O)[C@@H]2CSCN2)c(Cl)c(C(N)=O)c1]

In embodiments, the compound has the formula

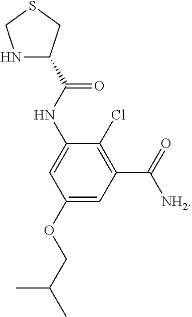 [SMILES: CC(C)COc1cc(NC(=O)[C@H]2CSCN2)c(Cl)c(C(N)=O)c1]

In embodiments, the compound has the formula

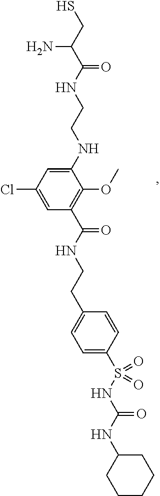 [SMILES: COc1c(NCCNC(=O)C(N)CS)cc(Cl)cc1C(=O)NCCc1ccc(S(=O)(=O)NC(=O)NC2CCCCC2)cc1]

In embodiments, the compound has the formula

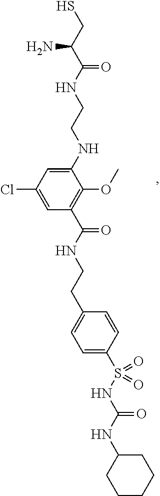 [SMILES: COc1c(NCCNC(=O)[C@@H](N)CS)cc(Cl)cc1C(=O)NCCc1ccc(S(=O)(=O)NC(=O)NC2CCCCC2)cc1]

In embodiments, the compound has the formula

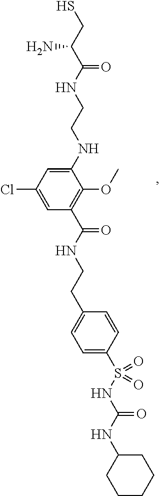 [SMILES: COc1c(NCCNC(=O)[C@H](N)CS)cc(Cl)cc1C(=O)NCCc1ccc(S(=O)(=O)NC(=O)NC2CCCCC2)cc1]

In embodiments, the compound has the formula

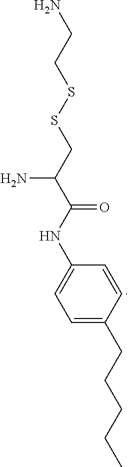 [SMILES: CCCCCc1ccc(NC(=O)C(N)CSSCCN)cc1]

In embodiments, the compound has the formula

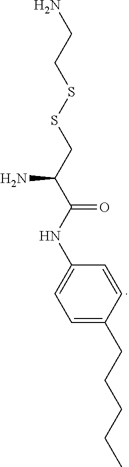 [SMILES: CCCCCc1ccc(NC(=O)[C@@H](N)CSSCCN)cc1]

In embodiments, the compound has the formula

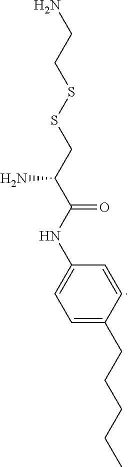 [SMILES: CCCCCc1ccc(NC(=O)[C@H](N)CSSCCN)cc1]

In embodiments, the compound has the formula

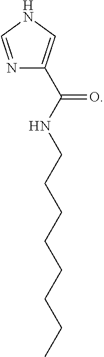 [SMILES: CCCCCCCCNC(=O)c1c[nH]cn1]

In embodiments, the compound has the formula

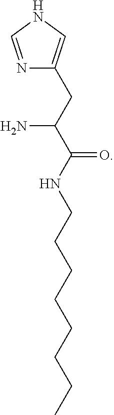 [SMILES: CCCCCCCCNC(=O)C(N)Cc1c[nH]cn1]

In embodiments, the compound has the formula

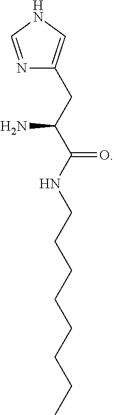 [SMILES: CCCCCCCCNC(=O)[C@@H](N)Cc1c[nH]cn1]

In embodiments, the compound has the formula

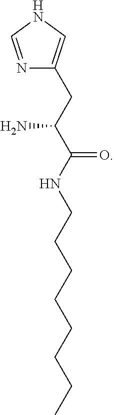 [SMILES: CCCCCCCCNC(=O)[C@H](N)Cc1c[nH]cn1]

In an aspect is provided a compound, or salt thereof, having the formula:

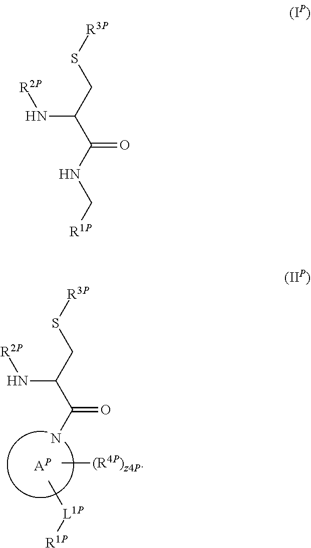

Ring A^{P} is a heterocycloalkyl or heteroaryl.

L^{1P} is L^{101P}-L^{102P}-L^{103P}.

L^{101P} is a bond, —S(O)₂—, —N(R^{101P})—, —O—, —S—, —C(O)—, —C(O)N(R^{101P})—, —N(R^{101P})C(O)—, —N(R^{101P})C(O)NH—, —NHC(O)N(R^{101P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{102P} is a bond, —S(O)₂—, —N(R^{102P})—, —O—, —S—, —C(O)—, —C(O)N(R^{102P})—, —N(R^{102P})C(O)—, —N(R^{102P})C(O)NH—, —NHC(O)N(R^{102P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{103P} is a bond, —S(O)₂—, —N(R^{103P})—, —O—, —S—, —C(O)—, —C(O)N(R^{103P})—, —N(R^{103P})C(O)—, —N(R^{103P})C(O)NH—, —NHC(O)N(R^{103P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R^{101P}, R^{102P}, and R^{103P} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl.

R^{1P} is hydrogen, halogen, —CX^{1P}₃, —CHX^{1P}₂, —CH₂X^{1P}, —OCX^{1P}₃, —OCH₂X^{1P}, —OCHX^{1P}₂, —CN, —SO_{n1P}R^{1DP}, —SO_{v1P}NR^{1AP}R^{1BP}, —NHC(O)NR^{1AP}R^{1BP}, —N(O)_{m1P}, —NR^{1AP}R^{1BP}, —C(O)R^{1CP}, —C(O)OR^{1CP}, —C(O)NR^{1AP}R^{1BP}, —OR^{1DP}, —NR^{1AP}SO₂R^{1DP}, —NR^{1AP}C(O)R^{1CP}, —NR^{1AP}C(O)OR^{1CP}, —NR^{1AP}OR^{1CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2P} is hydrogen, halogen, —CX^{2P}₃, —CHX^{2P}₂, —CH₂X^{2P}, —OCX^{2P}₃, —OCH₂X^{2P}, —OCHX^{2P}₂, —CN, —SO_{n2P}R^{2DP}, —SO_{v2P}NR^{2AP}R^{2BP}, —NHC(O)NR^{2AP}R^{2BP}, —N(O)_{m2P}, —NR^{2AP}R^{2BP}, —C(O)R^{2CP}, —C(O)OR^{2CP}, —C(O)NR^{2AP}R^{2BP}, —OR^{2DP}, —NR^{2AP}SO₂R^{2DP}, —NR^{2AP}C(O)R^{2CP}, —NR^{2AP}C(O)OR^{2CP}, —NR^{2AP}OR^{2CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3P} is hydrogen, halogen, —CX^{3P}₃, —CHX^{3P}₂, —CH₂X^{3P}, —OCX^{3P}₃, —OCH₂X^{3P}, —OCHX^{3P}₂, —CN, —SO_{n3P}R^{3DP}, —SO_{v3P}NR^{3AP}R^{3BP}, —NHC(O)NR^{3AP}R^{3BP}, —N(O)_{m3P}, —NR^{3AP}R^{3BP}, —C(O)R^{3CP}, —C(O)OR^{3CP}, —C(O)NR^{3AP}R^{3BP}, —OR^{3DP}, —NR^{3AP}SO₂R^{3DP}, —NR^{3AP}C(O)R^{3CP}, —NR^{3AP}C(O)OR^{3CP}, —NR^{3AP}OR^{3CP}, —N₃, —SR^{3AP}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1AP}, R^{1BP}, R^{1CP}, R^{1DP}, R^{2AP}, R^{2BP}, R^{2CP}, R^{2DP}, R^{3AP}, R^{3BP}, R^{3CP}, and R^{3DP} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1AP} and R^{1BP} substituents bonded to the same nitrogen atom may be joined to form a substituted or unsubstituted heterocycloalkyl or substituted or unsubstituted heteroaryl; R^{2AP} and R^{2BP} substituents bonded to the same nitrogen atom may be joined to form a substituted or unsubstituted heterocycloalkyl or substituted or unsubstituted heteroaryl; R^{3AP} and R^{3BP} substituents bonded to the same nitrogen atom may be joined to form a substituted or unsubstituted heterocycloalkyl or substituted or unsubstituted heteroaryl.

R^{2P} and R^{3P} substituents may be joined to form a substituted or unsubstituted heterocycloalkyl, or substituted or unsubstituted heteroaryl.

R^{4P} is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

n1P, n2P, and n3P are independently an integer from 0 to 4.

m1P, m2P, m3P, v1P, v2P, and v3P are independently 1 or 2.

X^{1P}, X^{2P}, and X^{3P} are independently —F, —Cl, —Br, or —I.

z4P is an integer from 0 to 6.

In embodiments, a substituted L^{1P} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{1P} is substituted with a plurality of groups selected from substituent groups, size-limited substituent groups, and lower substituent groups; each substituent group, size-limited substituent group, and/or lower substituent group may optionally be different. In embodiments, when L^{1P} is substituted, it is substituted with at least one substituent group. In embodiments, when L^{1P} is substituted, it is substituted with at least one size-limited substituent group. In embodiments, when L^{1P} is substituted, it is substituted with at least one lower substituent group.

In embodiments, L^{1P} is independently a bond. In embodiments, L^{1P} is independently an unsubstituted alkylene. In embodiments, L^{1P} is independently an unsubstituted phenylene.

In embodiments, L^{1P} is independently a bond. In embodiments, L^{1P} is independently an unsubstituted alkylene (e.g., C₁-C₈, C₁-C₆, C₁-C₄, or C₁-C₂). In embodiments, L^{1P} is independently an unsubstituted phenylene.

In embodiments, a substituted L^{101P} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{101P} is substituted with a plurality of groups selected from substituent groups, size-limited substituent groups, and lower substituent groups; each substituent group, size-limited substituent group, and/or lower substituent group may optionally be different. In embodiments, when L^{101P} is substituted, it is substituted with at least one substituent group. In embodiments, when L^{101P} is substituted, it is substituted with at least one size-limited substituent group. In embodiments, when L^{101P} is substituted, it is substituted with at least one lower substituent group.

In embodiments, L^{101P} is a bond, —S(O)₂—, —N(R^{101P})—, —O—, —S—, —C(O)—, —C(O)N(R^{101P})—, —N(R^{101P})C(O)—, —N(R^{101P})C(O)NH—, —NHC(O)N(R^{101P})—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{102P} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{102P} is substituted with a plurality of groups selected from substituent groups, size-limited substituent groups, and lower substituent groups; each substituent group, size-limited substituent group, and/or lower substituent group may optionally be different. In embodiments, when L^{102P} is substituted, it is substituted with at least one substituent group. In embodiments, when L^{102P} is substituted, it is substituted with at least one size-limited substituent group. In embodiments, when L^{102P} is substituted, it is substituted with at least one lower substituent group.

In embodiments, L^{102P} is a bond, —S(O)₂—, —N(R^{102P})—, —O—, —S—, —C(O)—, —C(O)N(R^{102P})—, —N(R^{102P})C(O)—, —N(R^{102P})C(O)NH—, —NHC(O)N(R^{102P})—, —C(O)O—, —C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{103P} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{103P} is substituted with a plurality of groups selected from substituent groups, size-limited substituent groups, and lower substituent groups; each substituent group, size-limited substituent group, and/or lower substituent group may optionally be different. In embodiments, when L^{103P} is substituted, it is substituted with at least one substituent group. In embodiments, when L^{103P} is substituted, it is substituted with at least one size-limited substituent group. In embodiments, when L^{103P} is substituted, it is substituted with at least one lower substituent group.

In embodiments, L^{103P} is a bond, —S(O)₂—, —N(R^{103P})—, —O—, —S—, —C(O)—, —C(O)N(R^{103P})—, —N(R^{103P})C(O)—, —N(R^{103P})C(O)NH—, —NHC(O)N(R^{103P})—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, R^{101P}, R^{102P}, and R^{103P} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R^{101P}, R^{102P}, and R^{103P} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, a substituted R^{1P} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1P} is substituted with a plurality of groups selected from substituent groups, size-limited substituent groups, and lower substituent groups; each substituent group, size-limited substituent group, and/or lower substituent group may optionally be different. In embodiments, when R^{1P} is substituted, it is substituted with at least one substituent group. In embodiments, when R^{1P} is substituted, it is substituted with at least one size-limited substituent group. In embodiments, when R^{1P} is substituted, it is substituted with at least one lower substituent group.

In embodiments, R^{1P} is hydrogen, halogen, —CX^{1P}₃, —CHX^{1P}₂, —CH₂X^{1P}, —OCX^{1P}₃, —OCH₂X^{1P}, —OCHX^{1P}₂, —CN, —SO_{n1P}R^{1DP}, —SO_{v1P}NR^{1AP}R^{1BP}, —NHC(O)NR^{1AP}R^{1BP}, —N(O)_{m1P}, —NR^{1AP}R^{1BP}, —C(O)R^{1CP}, —C(O)OR^{1CP}, —C(O)NR^{1AP}R^{1BP}, —OR^{1DP}, —NR^{1AP}SO₂R^{1DP}, —NR^{1AP}C(O)R^{1CP}, —NR^{1AP}C(O)OR^{1CP}, —NR^{1AP}OR^{1CP}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{1P} is independently a substituted or unsubstituted alkyl. In embodiments, R^{1P} is independently a substituted or unsubstituted C₄-C₁₂ alkyl. In embodiments, R^{1P} is independently a substituted or unsubstituted heteroalkyl. In embodiments, R^{1P} is independently a substituted or unsubstituted 4 to 12 membered heteroalkyl.

In embodiments, a substituted R^{1AP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1AP} is substituted with a plurality of groups selected from substituent groups, size-limited substituent groups, and lower substituent groups; each substituent group, size-limited substituent group, and/or lower substituent group may optionally be different. In embodiments, when R^{1AP} is substituted, it is substituted with at least one substituent group. In embodiments, when R^{1AP} is substituted, it is substituted with at least one size-limited substituent group. In embodiments, when R^{1AP} is substituted, it is substituted with at least one lower substituent group.

In embodiments, a substituted R^{1BP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1BP} is substituted with a plurality of groups selected from substituent groups, size-limited substituent groups, and lower substituent groups; each substituent group, size-limited substituent group, and/or lower substituent group may optionally be different. In embodiments, when R^{1BP} is substituted, it is substituted with at least one substituent group. In embodiments, when R^{1BP} is substituted, it is substituted with at least one size-limited substituent group. In embodiments, when R^{1BP} is substituted, it is substituted with at least one lower substituent group.

In embodiments, a substituted ring formed when R^{1AP} and R^{1BP} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1AP} and R^{1BP} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{1AP} and R^{1BP} substituents bonded to the same nitrogen atom are joined is substituted, it is substituted with at least one substituent group. In embodiments, when the ring formed when R^{1AP} and R^{1BP} substituents bonded to the same nitrogen atom are joined is substituted, it is substituted with at least one size-limited substituent group. In embodiments, when the ring formed when R^{1AP} and R^{1BP} substituents bonded to the same nitrogen atom are joined is substituted, it is substituted with at least one lower substituent group.

In embodiments, a substituted R^{1CP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1CP} is substituted with a plurality of groups selected from substituent groups, size-limited substituent groups, and lower substituent groups; each substituent group, size-limited substituent group, and/or lower substituent group may optionally be different. In embodiments, when R^{1CP} is substituted, it is substituted with at least one substituent group. In embodiments, when R^{1CP} is substituted, it is substituted with at least one size-limited substituent group. In embodiments, when R^{1CP} is substituted, it is substituted with at least one lower substituent group.

In embodiments, a substituted R^{1DP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1DP} is substituted with a plurality of groups selected from substituent groups, size-limited substituent groups, and lower substituent groups; each substituent group, size-limited substituent group, and/or lower substituent group may optionally be different. In embodiments, when R^{1DP} is substituted, it is substituted with at least one substituent group. In embodiments, when R^{1DP} is substituted, it is substituted with at least one size-limited substituent group. In embodiments, when R^{1DP} is substituted, it is substituted with at least one lower substituent group.

In embodiments, a substituted R^{2P} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2P} is substituted with a plurality of groups selected from substituent groups, size-limited substituent groups, and lower substituent groups; each substituent group, size-limited substituent group, and/or lower substituent group may optionally be different. In embodiments, when R^{2P} is substituted, it is substituted with at least one substituent group. In embodiments, when R^{2P} is substituted, it is substituted with at least one size-limited substituent group. In embodiments, when R^{2P} is substituted, it is substituted with at least one lower substituent group.

In embodiments, R^{2P} is hydrogen, halogen, —CX^{2P}₃, —CHX^{2P}₂, —CH₂X^{2P}, —OCX^{2P}₃, —OCH₂X^{2P}, —OCHX^{2P}₂, —CN, —SO_{n2P}R^{2DP}, —SO_{v2P}NR^{2AP}R^{2BP}, —NHC(O)NR^{2AP}R^{2BP}, —N(O)_{m2P}, —NR^{2AP}R^{2BP}, —C(O)R^{2CP}, —C(O)OR^{2CP}, —C(O)NR^{2AP}R^{2BP}, —OR^{2DP}, —NR^{2AP}SO₂R^{2DP}, —NR^{2AP}C(O)R^{2CP}, —NR^{2AP}C(O)OR^{2CP}, —NR^{2AP}OR^{2CP}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{2P} is independently hydrogen.

In embodiments, a substituted R^{2AP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2AP} is substituted with a plurality of groups selected from substituent groups, size-limited substituent groups, and lower substituent groups; each substituent group, size-limited substituent group, and/or lower substituent group may optionally be different. In embodiments, when R^{2AP} is substituted, it is substituted with at least one substituent group. In embodiments, when R^{2AP} is substituted, it is substituted with at least one size-limited substituent group. In embodiments, when R^{2AP} is substituted, it is substituted with at least one lower substituent group.

In embodiments, a substituted R^{2BP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2BP} is substituted with a plurality of groups selected from substituent groups, size-limited substituent groups, and lower substituent groups; each substituent group, size-limited substituent group, and/or lower substituent group may optionally be different. In embodiments, when R^{2BP} is substituted, it is substituted with at least one substituent group. In embodiments, when R^{2BP} is substituted, it is substituted with at least one size-limited substituent group. In embodiments, when R^{2BP} is substituted, it is substituted with at least one lower substituent group.

In embodiments, a substituted ring formed when R^{2AP} and R^{2BP} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2AP} and R^{2BP} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{2AP} and R^{2BP} substituents bonded to the same nitrogen atom are joined is substituted, it is substituted with at least one substituent group. In embodiments, when the ring formed when R^{2AP} and R^{2BP} substituents bonded to the same nitrogen atom are joined is substituted, it is substituted with at least one size-limited substituent group. In embodiments, when the ring formed when R^{2AP} and R^{2BP} substituents bonded to the same nitrogen atom are joined is substituted, it is substituted with at least one lower substituent group.

In embodiments, a substituted R^{2CP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2CP} is substituted with a plurality of groups selected from substituent groups, size-limited substituent groups, and lower substituent groups; each substituent group, size-limited substituent group, and/or lower substituent group may optionally be different. In embodiments, when R^{2CP} is substituted, it is substituted with at least one substituent group. In embodiments, when R^{2CP} is substituted, it is substituted with at least one size-limited substituent group. In embodiments, when R^{2CP} is substituted, it is substituted with at least one lower substituent group.

In embodiments, a substituted R^{2DP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2DP} is substituted with a plurality of groups selected from substituent groups, size-limited substituent groups, and lower substituent groups; each substituent group, size-limited substituent group, and/or lower substituent group may optionally be different. In embodiments, when R^{2DP} is substituted, it is substituted with at least one substituent group. In embodiments, when R^{2DP} is substituted, it is substituted with at least one size-limited substituent group. In embodiments, when R^{2DP} is substituted, it is substituted with at least one lower substituent group.

In embodiments, a substituted R^{3P} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3P} is substituted with a plurality of groups selected from substituent groups, size-limited substituent groups, and lower substituent groups; each substituent group, size-limited substituent group, and/or lower substituent group may optionally be different. In embodiments, when R^{3P} is substituted, it is substituted with at least one substituent group. In embodiments, when R^{3P} is substituted, it is substituted with at least one size-limited substituent group. In embodiments, when R^{3P} is substituted, it is substituted with at least one lower substituent group.

In embodiments, R^{3P} is hydrogen, halogen, —CX^{3P}₃, —CHX^{3P}₂, —CH₂X^{3P}, —OCX^{3P}₃, —OCH₂X^{3P}, —OCHX^{3P}₂, —CN, —SO_{n3P}R^{3DP}, —SO_{v3P}NR^{3AP}R^{3BP}, —NHC(O)NR^{3AP}R^{3BP}, —N(O)_{m3P}, —NR^{3AP}R^{3BP}, —C(O)R^{3CP}, —C(O)OR^{3CP}, —C(O)NR^{3AP}R^{3BP}, —OR^{3DP}, —NR^{3AP}SO₂R^{3DP}, —NR^{3AP}C(O)R^{3CP}, —NR^{3AP}C(O)OR^{3CP}, —NR^{3AP}OR^{3CP}, —N₃, —SR^{3AP}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{3P} is independently hydrogen. In embodiments, R³ is independently —SR^{3AP}. In embodiments, R^{3AP} is independently an unsubstituted heteroaryl. In embodiments, R^{3AP} is independently an unsubstituted 2-pyridyl. In embodiments, R^{3AP} is independently unsubstituted tert-butyl. In embodiments, R^{3AP} is independently —CH₂CH₂NH₂.

In embodiments, a substituted R^{3AP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3AP} is substituted with a plurality of groups selected from substituent groups, size-limited substituent groups, and lower substituent groups; each substituent group, size-limited substituent group, and/or lower substituent group may optionally be different. In embodiments, when R^{3AP} is substituted, it is substituted with at least one substituent group. In embodiments, when R^{3AP} is substituted, it is substituted with at least one size-limited substituent group. In embodiments, when R^{3AP} is substituted, it is substituted with at least one lower substituent group.

In embodiments, a substituted R^{3BP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3BP} is substituted with a plurality of groups selected from substituent groups, size-limited substituent groups, and lower substituent groups; each substituent group, size-limited substituent group, and/or lower substituent group may optionally be different. In embodiments, when R^{3BP} is substituted, it is substituted with at least one substituent group. In embodiments, when R^{3BP} is substituted, it is substituted with at least one size-limited substituent group. In embodiments, when R^{3BP} is substituted, it is substituted with at least one lower substituent group.

In embodiments, a substituted ring formed when R^{3AP} and R^{3BP} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3AP} and R^{3BP} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{3AP} and R^{3BP} substituents bonded to the same nitrogen atom are joined is substituted, it is substituted with at least one substituent group. In embodiments, when the ring formed when R^{3AP} and R^{3BP} substituents bonded to the same nitrogen atom are joined is substituted, it is substituted with at least one size-limited substituent group. In embodiments, when the ring formed when R^{3AP} and R^{3BP} substituents bonded to the same nitrogen atom are joined is substituted, it is substituted with at least one lower substituent group.

In embodiments, a substituted R^{3CP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3CP} is substituted with a plurality of groups selected from substituent groups, size-limited substituent groups, and lower substituent groups; each substituent group, size-limited substituent group, and/or lower substituent group may optionally be different. In embodiments, when R^{3CP} is substituted, it is substituted with at least one substituent group. In embodiments, when R^{3CP} is substituted, it is substituted with at least one size-limited substituent group. In embodiments, when R^{3CP} is substituted, it is substituted with at least one lower substituent group.

In embodiments, a substituted R^{3DP} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3DP} is substituted with a plurality of groups selected from substituent groups, size-limited substituent groups, and lower substituent groups; each substituent group, size-limited substituent group, and/or lower substituent group may optionally be different. In embodiments, when R^{3DP} is substituted, it is substituted with at least one substituent group. In embodiments, when R^{3DP} is substituted, it is substituted with at least one size-limited substituent group. In embodiments, when R^{3DP} is substituted, it is substituted with at least one lower substituent group.

In embodiments, R^{1AP}, R^{1BP}, R^{1CP}, R^{1DP}, R^{2AP}, R^{2BP}, R^{2CP}, R^{2DP}, R^{3AP}, R^{3BP}, R^{3CP}, and R^{3DP} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1AP} and R^{1BP} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2AP} and R^{2BP} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3AP} and R^{3BP} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, a substituted ring formed when R^{2P} and R^{3P} substituents are joined (e.g., substituted heterocycloalkyl and/or substituted heteroaryl) is substituted with at least one substituent group, size-limited substituent group, or lower substituent group; wherein if the substituted ring formed when R^{2P} and R^{3P} substituents are joined is substituted with a plurality of groups selected from substituent groups, size-limited substituent groups, and lower substituent groups; each substituent group, size-limited substituent group, and/or lower substituent group may optionally be different. In embodiments, when the ring formed when R^{2P} and R^{3P} substituents are joined is substituted, it is substituted with at least one substituent group. In embodiments, when the ring formed when R^{2P} and R^{3P} substituents are joined is substituted, it is substituted with at least one size-limited substituent group. In embodiments, when the ring formed when R^{2P} and R^{3P} substituents are joined is substituted, it is substituted with at least one lower substituent group.

In embodiments, R^{2P} and R^{3P} substituents may be joined to form a substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered).

In embodiments, a substituted R^{4P} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4P} is substituted with a plurality of groups selected from substituent groups, size-limited substituent groups, and lower substituent groups; each substituent group, size-limited substituent group, and/or lower substituent group may optionally be different. In embodiments, when R^{4P} is substituted, it is substituted with at least one substituent group. In embodiments, when R^{4P} is substituted, it is substituted with at least one size-limited substituent group. In embodiments, when R^{4P} is substituted, it is substituted with at least one lower substituent group.

In embodiments, R^{4P} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{4P} is halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{4P} is independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{4P} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{4P} is halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound described herein (e.g., in the Compounds section, Examples Section, Methods Section, or in a claim, table, or figure).

### III. Pharmaceutical Compositions

In an aspect is provided a pharmaceutical composition including a compound described herein and a pharmaceutically acceptable excipient.

In embodiments, the pharmaceutical composition includes an effective amount of the compound. In embodiments, the pharmaceutical composition includes a therapeutically effective amount of the compound.

In embodiments, the pharmaceutical composition includes an effective amount of a second agent, wherein the second agent is an anti-cancer agent. In embodiments, the pharmaceutical composition includes the second agent in a therapeutically effective amount.

### IV. Methods

In an aspect is provided a method of treating a depalmitoylation-associated disease in a subject in need thereof, the method including administering to the subject an effective amount of a compound described herein. In embodiments, the method includes administering to the subject a therapeutically effective amount of a compound described herein.

In embodiments, the depalmitoylation-associated disease is a cancer, neurodegenerative disease, developmental disease, autoimmune disease, inflammatory disease, or infectious disease. In embodiments, the depalmitoylation-associated disease is a cancer or a neurodegenerative disease. In embodiments, the depalmitoylation-associated disease is a cancer. In embodiments, the depalmitoylation-associated disease is a neurodegenerative disease. In embodiments, the depalmitoylation-associated disease is a developmental disease. In embodiments, the depalmitoylation-associated disease is an autoimmune disease. In embodiments, the depalmitoylation-associated disease is an inflammatory disease. In embodiments, the depalmitoylation-associated disease is an infectious disease.

In embodiments, the depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis, or glioma. In embodiments, the depalmitoylation-associated disease is bladder cancer. In embodiments, the depalmitoylation-associated disease is head and neck cancer. In embodiments, the depalmitoylation-associated disease is Costello's Syndrome. In embodiments, the depalmitoylation-associated disease is melanoma. In embodiments, the depalmitoylation-associated disease is acute myeloid lymphoma (AML). In embodiments, the depalmitoylation-associated disease is non-small cell lung carcinoma. In embodiments, the depalmitoylation-associated disease is Alzheimer's disease. In embodiments, the depalmitoylation-associated disease is infantile neuronal ceroid lipofuscinosis. In embodiments, the depalmitoylation-associated disease is glioma.

In an aspect is provided a method of treating a disease, the method including administering to a subject in need thereof an effective amount of a compound described herein. In embodiments, the method includes administering to a subject in need thereof a therapeutically effective amount of a compound described herein. In embodiments, the disease is a cancer, a CNS disease, or a developmental disease.

In embodiments, the disease is a cancer. In embodiments, the cancer is bladder cancer, head and neck cancer, Costello's Syndrome, melanoma, acute myeloid lymphoma (AML), non-small cell lung carcinoma, Alzheimer's disease, infantile neuronal ceroid lipofuscinosis or glioma. In embodiments depalmitoylation-associated disease is bladder cancer. In embodiments depalmitoylation-associated disease is head and neck cancer. In embodiments depalmitoylation-associated disease is Costello's Syndrome. In embodiments depalmitoylation-associated disease is melanoma. In embodiments depalmitoylation-associated disease is acute myeloid lymphoma (AML). In embodiments depalmitoylation-associated disease is non-small cell lung carcinoma. In embodiments depalmitoylation-associated disease is Alzheimer's disease. In embodiments depalmitoylation-associated disease is infantile neuronal ceroid lipofuscinosis. In embodiments depalmitoylation-associated disease is glioma.

In embodiments, the disease is a CNS disease. In embodiments, the CNS disease is Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

In embodiments, the disease is a developmental disease. In embodiments, the developmental disease is developmental language disorder, learning disorder, motor disorder, or autism spectrum disorder.

In another aspect, a method of depalmitoylating a protein in a cell is provided. The method includes contacting the cell with an effective amount of a compound described herein including embodiments thereof. In embodiments the protein forms part of the plasma membrane of the cell. In embodiments, the protein is HRas or NRas. In embodiments the protein is HRas. In embodiments the protein is NRas.

In embodiments, the protein is HRas, NRas, EGFR, amyloid precursor protein (APP), BACE1, EZH2, PD-L1, flotillin-1, flotillin-2, calnexin, Gα(i), metadherin, CD44, or SNAP25. In embodiments, the protein is HRas. In embodiments, the protein is NRas. In embodiments, the protein is EGFR. In embodiments, the protein is amyloid precursor protein (APP). In embodiments, the protein is BACE1. In embodiments, the protein is EZH2. In embodiments, the protein is PD-L1. In embodiments, the protein is flotillin-1. In embodiments, the protein is flotillin-2. In embodiments, the protein is calnexin. In embodiments, the protein is Gα(i). In embodiments, the protein is metadherin. In embodiments, the protein is CD44. In embodiments, the protein is SNAP25.

In embodiments, the contacting occurs in vitro or in vivo. In embodiments, the contacting occurs in vitro. In embodiments, the contacting occurs in vivo. In embodiments, the cell forms part of an organism. In embodiments, the cell forms part of a mammalian subject. In embodiments, the mammalian subject suffers from a depalmitoylation-associated disease. In embodiments, the mammalian subject suffers from a cancer or a neurodegenerative disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. Embodiments

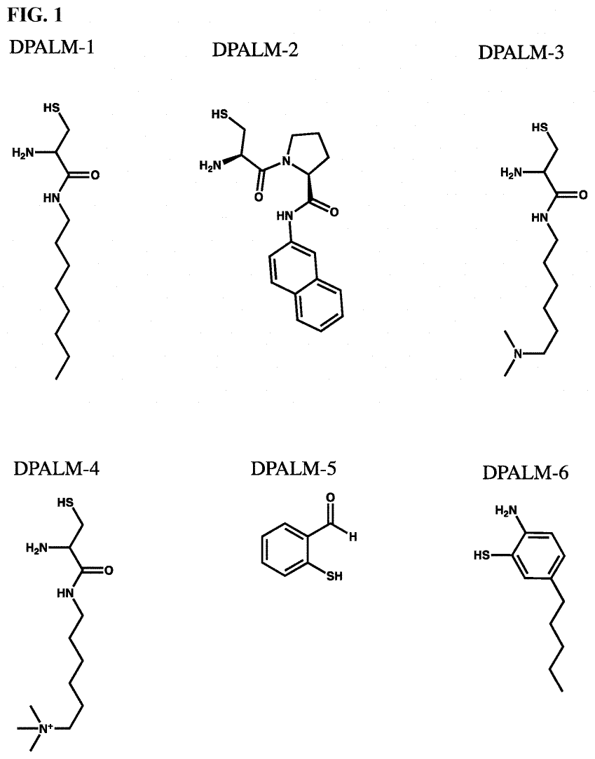

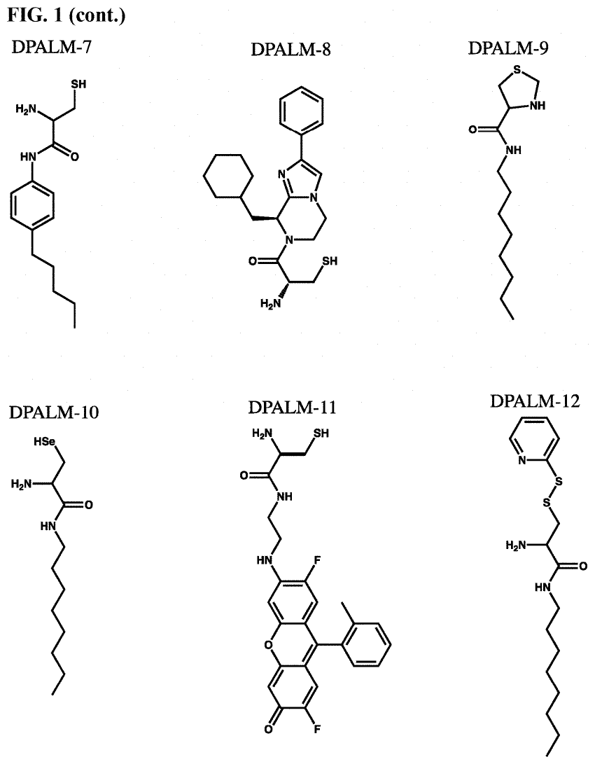

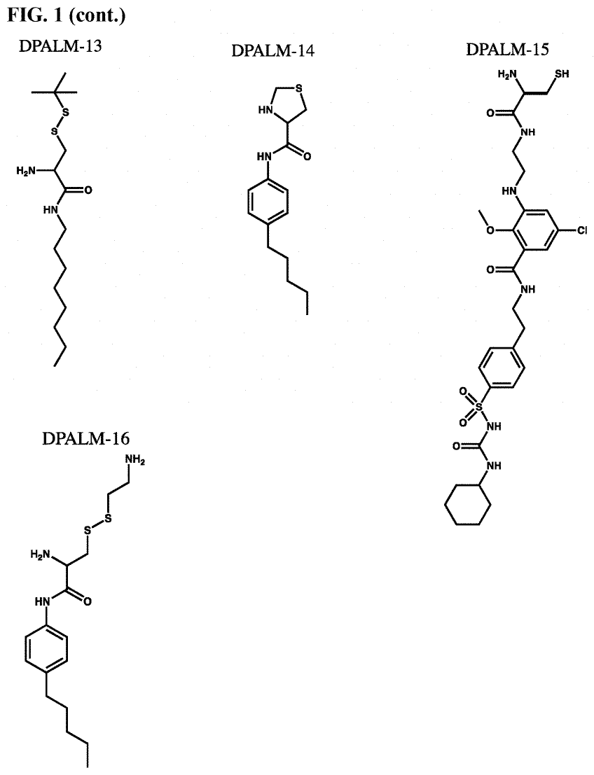

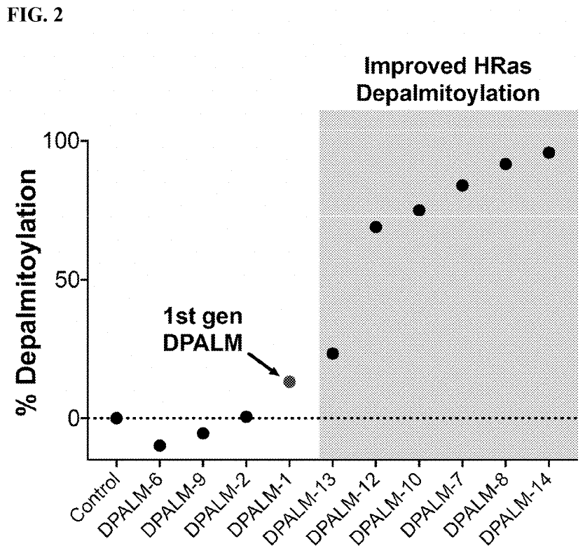

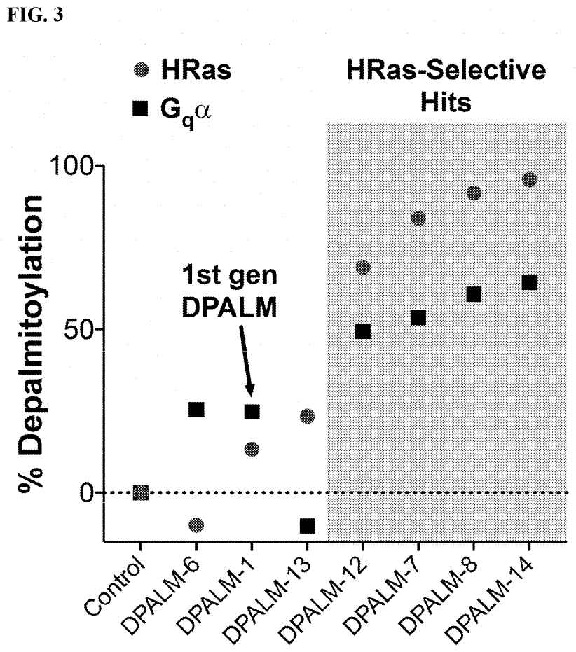

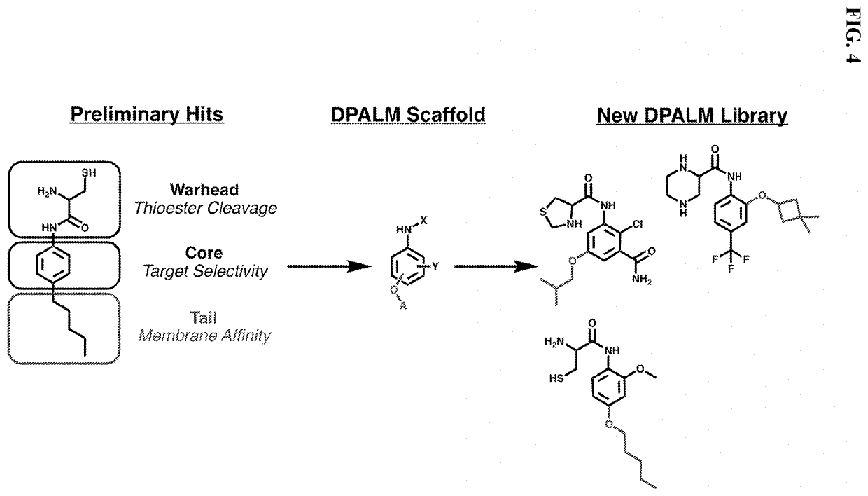

Embodiment P1. A compound, or salt thereof, having a formula as shown in FIG. 1, or having the formula

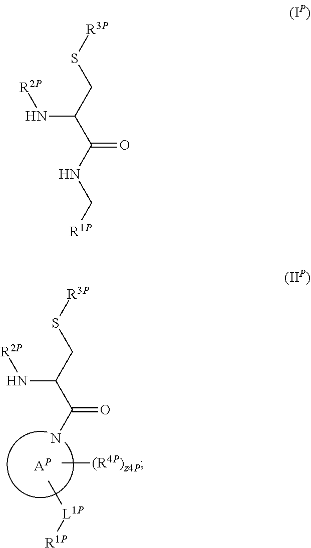

wherein
Ring A^{P} is a heterocycloalkyl or heteroaryl;
L^{1P} is L^{101P}-L^{102P}-L^{103P};
L^{101P} is a bond, —S(O)₂—, —N(R^{101P})—, —O—, —S—, —C(O)—, —C(O)N(R^{101P})—, —N(R^{101P})C(O)—, —N(R^{101P})C(O)NH—, —NHC(O)N(R^{101P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{102P} is a bond, —S(O)₂—, —N(R^{102P})—, —O—, —S—, —C(O)—, —C(O)N(R^{102P})—, —N(R^{102P})C(O)—, —N(R^{102P})C(O)NH—, —NHC(O)N(R^{102P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{103P} is a bond, —S(O)₂—, —N(R^{103P})—, —O—, —S—, —C(O)—, —C(O)N(R^{103P})—, —N(R^{103P})C(O)—, —N(R^{103P})C(O)NH—, —NHC(O)N(R^{103P})—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{101P}, R^{102P}, and R^{103P} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl, unsubstituted heteroalkyl, unsubstituted cycloalkyl, unsubstituted heterocycloalkyl, unsubstituted aryl, or unsubstituted heteroaryl.
R^{1P} is hydrogen, halogen, —CX^{1P}₃, —CHX^{1P}₂, —CH₂X^{1P}, —OCX^{1P}₃, —OCH₂X^{1P}, —OCHX^{1P}₂, —CN, —SO_{n1P}R^{1DP}, SO_{v1P}NR^{1AP}R^{1BP}, —NHC(O)NR^{1AP}R^{1BP}, —N(O)_{m1P}, —NR^{1AP}R^{1BP}, —C(O)R^{1CP}, —C(O)OR^{1CP}, —C(O)NR^{1AP}R^{1BP}, —OR^{1DP}, —NR^{1AP}SO₂R^{1DP}, —NR^{1AP}C(O)R^{1CP}, —NR^{1AP}C(O)OR^{1CP}, —NR^{1AP}OR^{1CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R^{2P} is hydrogen, halogen, —CX^{2P}₃, —CHX^{2P}₂, —CH₂X^{2P}, —OCX^{2P}₃, —OCH₂X^{2P}, —OCHX^{2P}₂, —CN, —SO_{n2P}R^{2DP}, —SO_{v2P}NR^{2AP}R^{2BP}, —NHC(O)NR^{2AP}R^{2BP}, —N(O)_{m2P}, —NR^{2AP}R^{2BP}, —C(O)R^{2CP}, —C(O)OR^{2CP}, —C(O)NR^{2AP}R^{2BP}, —OR^{2DP}, —NR^{2AP}SO₂R^{2DP}, —NR^{2AP}C(O)R^{2CP}, —NR^{2AP}C(O)OR^{2CP}, —NR^{2AP}OR^{2CP}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3P} is hydrogen, halogen, —CX^{3P}₃, —CHX^{3P}₂, —CH₂X^{3P}, —OCX^{3P}₃, —OCH₂X^{3P}, —OCHX^{3P}₂, —CN, —SO_{n3P}R^{3DP}, SO_{v3P}NR^{3AP}R^{3BP}, —NHC(O)NR^{3AP}R^{3BP}, —N(O)_{m3P}, —NR^{3AP}R^{3BP}, —C(O)R³CP, —C(O)OR^{3CP}, —C(O)NR^{3AP}R^{3BP}, —OR^{3DP}, —NR^{3AP}SO₂R^{3DP}, —NR^{3AP}C(O)R^{3CP}, —NR^{3AP}C(O)OR^{3CP}, —NR^{3AP}OR^{3CP}, —N₃, —SR^{3AP}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1AP}, R^{1BP}, R^{1CP}, R^{1DP}, R^{2AP}, R^{2BP}, R^{2CP}, R^{2DP}, R^{3AP}, R^{3BP}, R^{3CP}, and R^{3DP} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1AP} and R^{1BP} substituents bonded to the same nitrogen atom may be joined to form a substituted or unsubstituted heterocycloalkyl or substituted or unsubstituted heteroaryl; R^{2AP} and R^{2BP} substituents bonded to the same nitrogen atom may be joined to form a substituted or unsubstituted heterocycloalkyl or substituted or unsubstituted heteroaryl; R^{3AP} and R^{3BP} substituents bonded to the same nitrogen atom may be joined to form a substituted or unsubstituted heterocycloalkyl or substituted or unsubstituted heteroaryl;
R^{2P} and R^{3P} substituents may be joined to form a substituted or unsubstituted heterocycloalkyl, or substituted or unsubstituted heteroaryl;
R^{4P} is halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
n1P, n2P, and n3P are independently an integer from 0 to 4;
m1P, m2P, m3P, v1P, v2P, and v3P are independently 1 or 2;
X^{1P}, X^{2P}, and X^{3P} are independently —F, —Cl, —Br, or —I; and
z4P is an integer from 0 to 6.

Embodiment P2. A pharmaceutical composition comprising a compound of embodiment P1, or pharmaceutically acceptable salt thereof, and a pharmaceutically acceptable excipient.

Embodiment P3. A method of treating a disease, said method comprising administering to a subject in need thereof an effective amount of a compound of embodiment P1, wherein the disease is a cancer, a CNS disease, or a developmental disease.

### VI. Additional Embodiments

Embodiment 1. A compound, or a pharmaceutically acceptable salt thereof, having the formula:

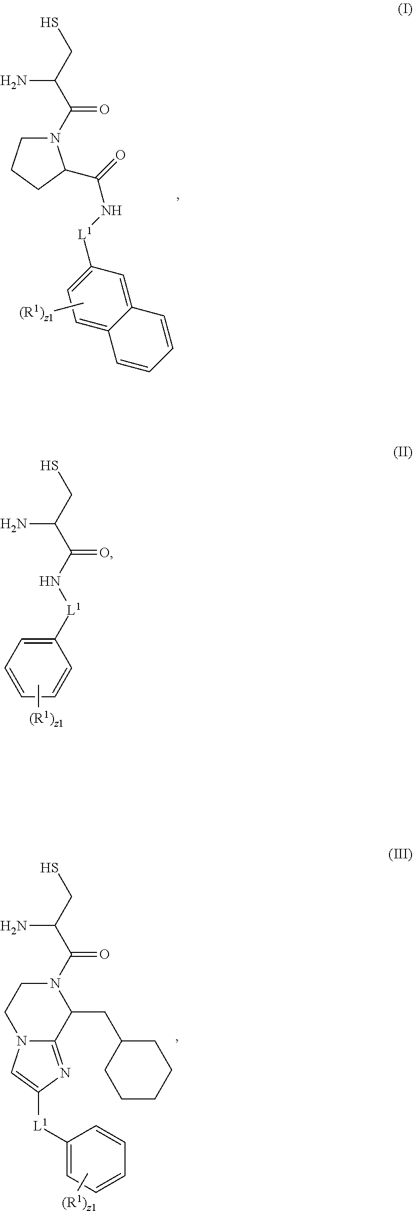

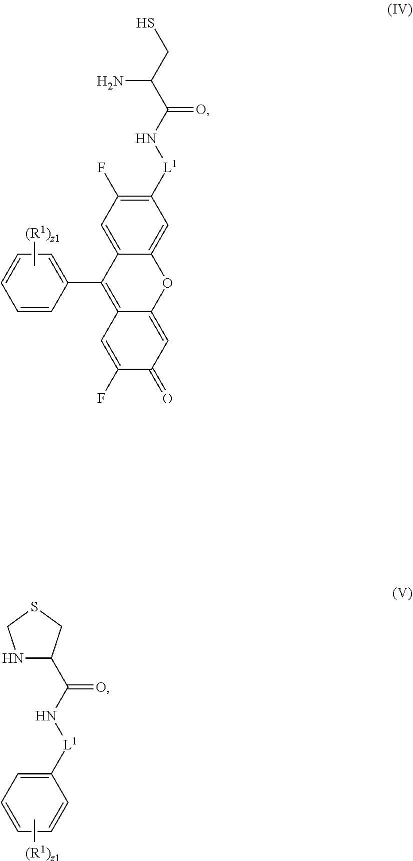

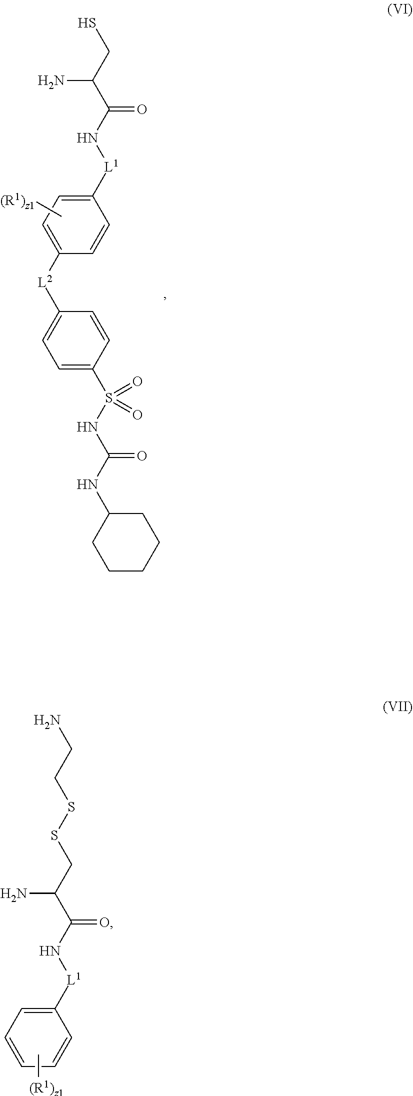

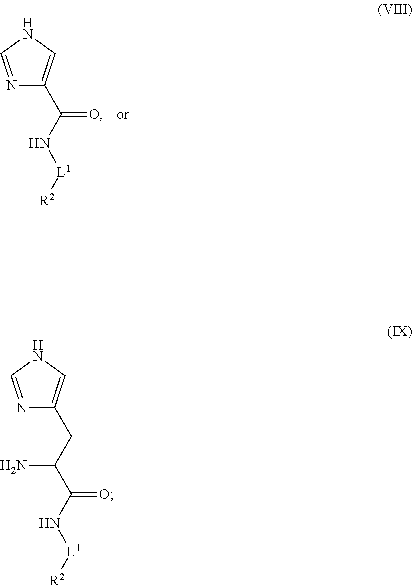

wherein
L¹ is a bond, substituted or unsubstituted C₁-C₁₀ alkylene, or substituted or unsubstituted 2 to 10 membered heteroalkylene;
L² is a bond, substituted or unsubstituted C₁-C₁₀ alkylene, or substituted or unsubstituted 2 to 10 membered heteroalkylene;
R¹ is independently a halogen, substituted or unsubstituted C₁-C₂₀ alkyl, or substituted or unsubstituted 2 to 20 membered heteroalkyl;
R² is hydrogen, halogen, substituted or unsubstituted C₁-C₂₀ alkyl, or substituted or unsubstituted 2 to 20 membered heteroalkyl; and
z1 is an integer from 0 to 7.

Embodiment 2. The compound of embodiment 1, wherein L¹ is a bond, unsubstituted C₁-C₁₀ alkylene, or unsubstituted 2 to 10 membered heteroalkylene.

Embodiment 3. The compound of embodiment 1, wherein L¹ is a bond.

Embodiment 4. The compound of embodiment 1, wherein L¹ is -(unsubstituted C₁-C₆ alkylene)-NH—.

Embodiment 5. The compound of embodiment 1, wherein L¹ is —CH₂—CH₂—NH—.

Embodiment 6. The compound of one of embodiments 1 to 5, wherein L² is a bond or oxo-substituted 2 to 10 membered heteroalkylene.

Embodiment 7. The compound of one of embodiments 1 to 5, wherein L² is —C(O)NH-(unsubstituted C₁-C₆ alkylene)-.

Embodiment 8. The compound of one of embodiments 1 to 5, wherein L² is —C(O)NH—CH₂—CH₂—.

Embodiment 9. The compound of one of embodiments 1 to 8, wherein R¹ is independently a halogen, unsubstituted C₁-C₁₀ alkyl, or substituted or unsubstituted 2 to 10 membered heteroalkyl.

Embodiment 10. The compound of one of embodiments 1 to 8, wherein R¹ is independently a halogen, unsubstituted C₁-C₆ alkyl, or unsubstituted C₁-C₆ alkoxy.

Embodiment 11. The compound of one of embodiments 1 to 8, wherein R¹ is independently —Cl.

Embodiment 12. The compound of one of embodiments 1 to 8, wherein R¹ is independently an unsubstituted C₁-C₆ alkyl.

Embodiment 13. The compound of one of embodiments 1 to 8, wherein R¹ is independently an unsubstituted C₁-C₆ alkoxy.

Embodiment 14. The compound of one of embodiments 1 to 8, wherein z1 is 0.

Embodiment 15. The compound of one of embodiments 1 to 13, wherein z1 is 1, 2, or 3.

Embodiment 16. The compound of one of embodiments 1 to 5, wherein R² is an unsubstituted C₁-C₂₀ alkyl, or unsubstituted 2 to 20 membered heteroalkyl.

Embodiment 17. The compound of one of embodiments 1 to 5, wherein R² is an unsubstituted C₁-C₁₀ alkyl.

Embodiment 18. The compound of embodiment 1, having the formula:

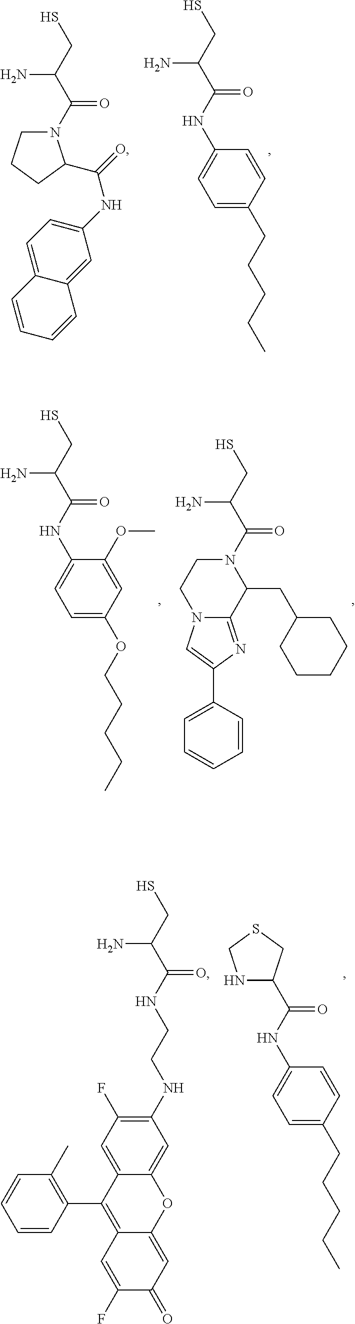

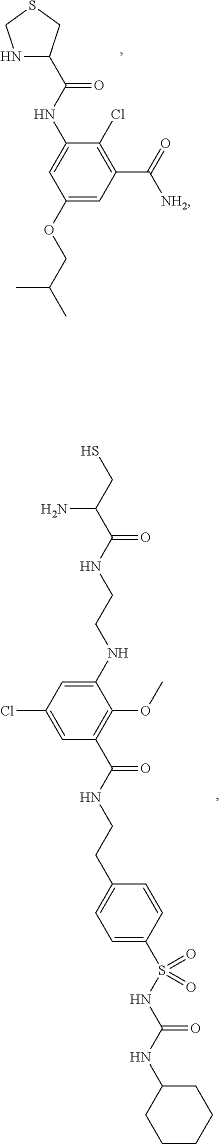 [SMILES: CC(C)COc1cc(NC(=O)C2CSCN2)c(Cl)c(C(N)=O)c1 ; COc1c(NCCNC(=O)C(N)CS)cc(Cl)cc1C(=O)NCCc1ccc(S(=O)(=O)NC(=O)NC2CCCCC2)cc1]

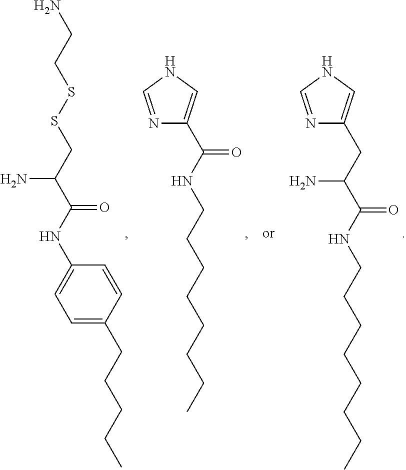 [SMILES: CCCCCc1ccc(NC(=O)C(N)CSSCCN)cc1 ; CCCCCCCCNC(=O)c1c[nH]cn1 ; CCCCCCCCNC(=O)C(N)Cc1c[nH]cn1]

Embodiment 19. A pharmaceutical composition comprising a compound of one of embodiments 1 to 18, or pharmaceutically acceptable salt thereof, and a pharmaceutically acceptable excipient.

Embodiment 20. A method of treating a depalmitoylation-associated disease in a subject in need thereof, said method comprising administering to the subject a therapeutically effective amount of a compound of one of embodiments 1 to 18.

Embodiment 21. The method of embodiment 20, wherein the depalmitoylation-associated disease is a cancer or a neurodegenerative disease.

Embodiment 22. The method of one of embodiments 20 to 21, wherein the depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis, or glioma.

Embodiment 23. A method of depalmitoylating a protein in a cell, said method comprising contacting the cell with an effective amount of a compound of one of embodiments 1 to 18.

Embodiment 24. The method of embodiment 23, wherein the protein forms part of the plasma membrane of the cell.

Embodiment 25. The method of embodiment 23, wherein the protein is HRas, NRas, EGFR, amyloid precursor protein (APP), BACE1, EZH2, PD-L1, flotillin-1, flotillin-2, calnexin, Gα(i), metadherin, CD44, or SNAP25.

Embodiment 26. The method of one of embodiments 23 to 25, wherein said contacting occurs in vitro or in vivo.

Embodiment 27. The method of one of embodiments 23 to 26, wherein the cell forms part of an organism.

Embodiment 28. The method of one of embodiments 23 to 26, wherein the cell forms part of a mammalian subject.

Embodiment 29. The method of embodiment 28, wherein said mammalian subject suffers from a cancer or a neurodegenerative disease.

### EXAMPLES

### Example 1: HRas-Selective Depalmitoylating Compounds

The ras genes have GTP/GDP binding and GTPase activity, and their normal function may be as G-like regulatory proteins involved in the normal control of cell growth. HRAS has been shown to be a proto-oncogene. When mutated, proto-oncogenes have the potential to cause normal cells to become cancerous. Mutations in the HRAS gene also have been associated with the progression of bladder cancer and an increased risk of tumor recurrence after treatment. Somatic mutations in the HRAS gene are probably involved in the development of several other types of cancer. These mutations lead to an HRas protein that is always active and can direct cells to grow and divide without control. NRas is an enzyme that in humans is encoded by the NRAS gene and was named NRAS, for its initial identification in human neuroblastoma cells.

The disclosed compounds show improved potency and selectivity toward the therapeutically relevant proteins HRas and NRas. Additionally, these compounds illustrate a general roadmap for the design and synthesis of depalmitoylating compounds with selectivity toward many different S-palmitoylated protein targets.

These compounds may show increased selectivity for HRas and NRas due to interactions between the molecule core and protein residues flanking the site of S-palmitoylation. By introducing different substituents into the molecule core, it may be possible to promote favorable interactions between these flanking residues and the depalmitoylating drug in a wide variety of protein targets.

We have conceived of a general strategy for the synthesis and screening of depalmitoylating small molecules with selectivity toward any S-palmitoylated protein.

These compounds show improved potency and selectivity toward the therapeutically relevant proteins HRas and NRas. Additionally, these compounds illustrate a general roadmap for the design and synthesis of depalmitoylating compounds with selectivity toward many different S-palmitoylated protein targets.

These compounds show increased selectivity for HRas and NRas due to interactions between the molecule core and protein residues flanking the site of S-palmitoylation. By introducing different substituents into the molecule core, it may be possible to promote favorable interactions between these flanking residues and the depalmitoylating drug in a wide variety of protein targets.

### Example 2: Experimental Procedures and Characterization Data

DPALM-1. Synthesis previously disclosed.

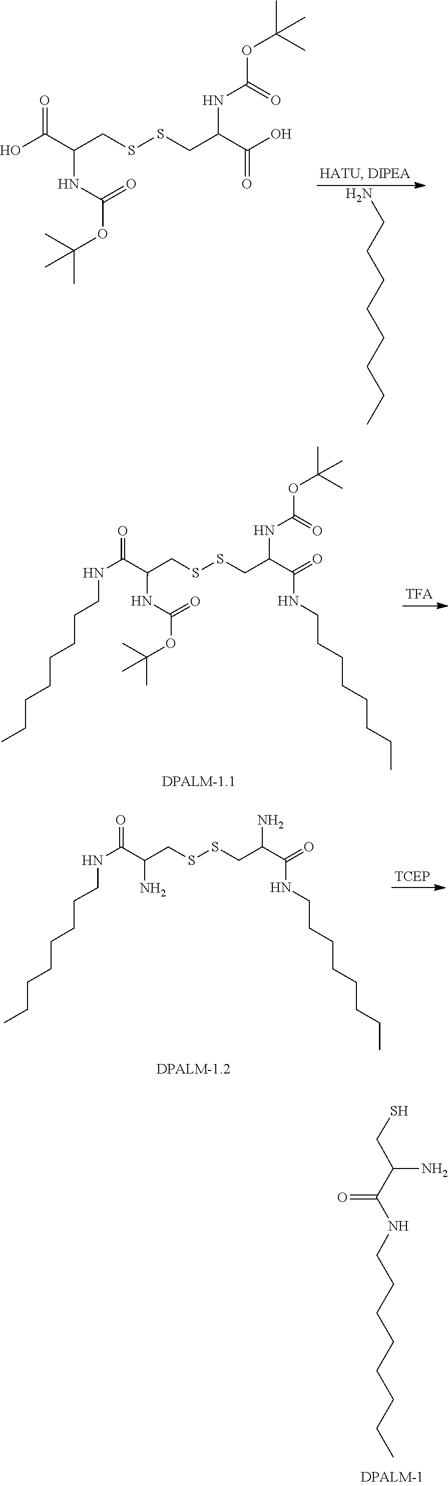

Synthesis of DPALM-2

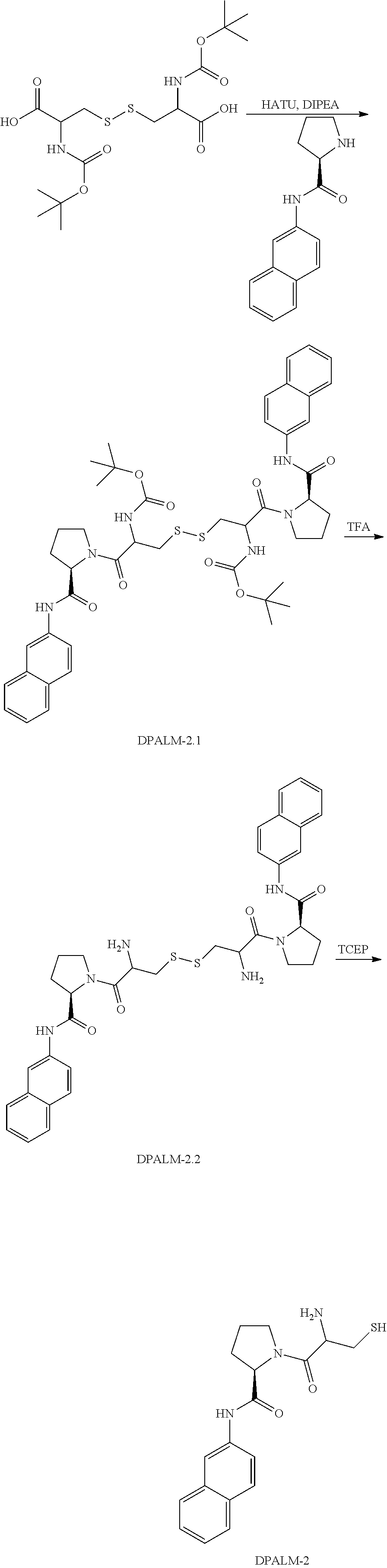 [SMILES: CC(C)(C)OC(=O)NC(CSSCC(NC(=O)OC(C)(C)C)C(=O)O)C(=O)O ; O=C(Nc1ccc2ccccc2c1)[C@H]1CCCN1 ; CC(C)(C)OC(=O)NC(CSSCC(NC(=O)OC(C)(C)C)C(=O)N1CCC[C@@H]1C(=O)Nc1ccc2ccccc2c1)C(=O)N1CCC[C@@H]1C(=O)Nc1ccc2ccccc2c1 ; NC(CSSCC(N)C(=O)N1CCC[C@@H]1C(=O)Nc1ccc2ccccc2c1)C(=O)N1CCC[C@@H]1C(=O)Nc1ccc2ccccc2c1 ; NC(CS)C(=O)N1CCC[C@@H]1C(=O)Nc1ccc2ccccc2c1]

DPALM-2.1. A solution of (Boc-Cys-OH)₂ (15 mg, 34.1 μmol) in CH₂Cl₂ (1 mL) was stirred at 0° C. for 10 min. HATU (42.7 mg, 112.4 μmol) and DIEA (71.2 μL, 408.6 μmol) were then added and the reaction stirred at 0° C. for 10 min. (S)—N-(naphthalen-2-yl)pyrrolidine-2-carboxamide (24.6 mg, 102.2 μmol) was then added and the reaction stirred for 1 h at rt. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography (1:9 MeOH:CH₂Cl₂) to afford 21.9 mg of DPALM-2.1 as a pale yellow solid (73% yield).

DPALM-2. A solution of DPALM-2.1 (21.6 mg, 24.4 μmol) in 1 mL of TFA/CH₂Cl₂ (1:1) was stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield 9.1 mg of DPALM-2.2 as a pale yellow solid (54% yield). DPALM-2.2 was then dissolved in DMSO containing 100 mM TCEP to yield the free thiol DPALM-2 in quantitative yield.

Synthesis of DPALM-3

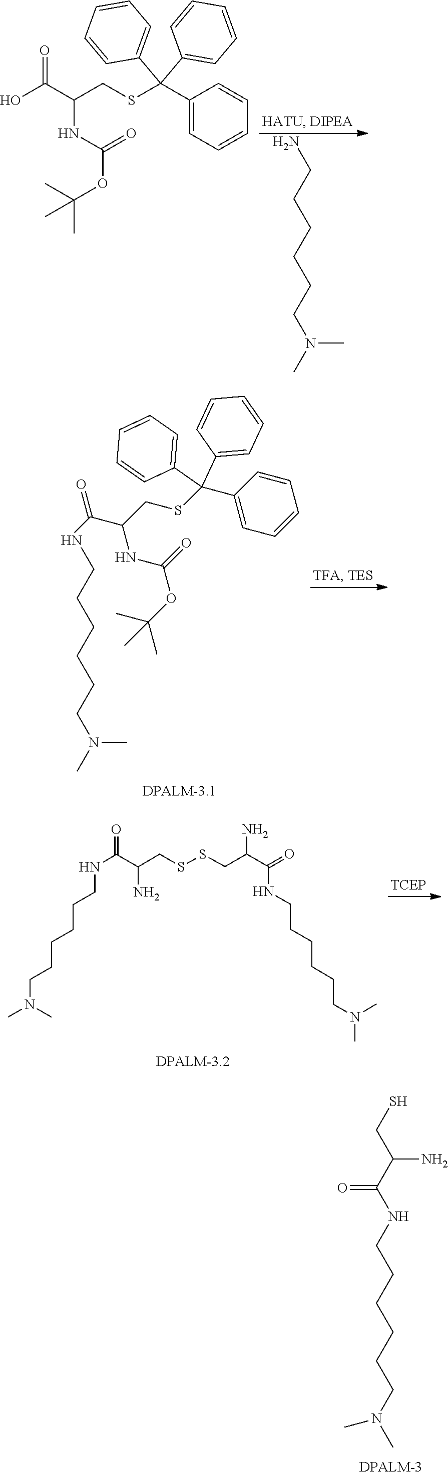

DPALM-3.1. A solution of N-Boc-L-Cys(Trt)-OH (250.0 mg, 539.3 μmol) in CH₂Cl₂ (5 mL) was stirred at 0° C. for 10 min, and then HATU (225.6 mg, 593.2 μmol) and DIEA (375.7 μL, 2.16 mmol) were successively added. After 10 min stirring at 0° C., 6-(dimethylamino)hexylamine (DMHA, 97.2 μL, 539.3 μmol) was added. After 1 h stirring at rt, the reaction mixture was concentrated, providing a yellow oil. Then, the corresponding residue was diluted in MeOH (500 μL), filtered using a 0.2 μm syringe-driven filter, and the crude solution was purified by HPLC, affording 295.1 mg of DPALM-3.1 as a colorless oil [93%, t_{R}=10.1 min (Zorbax SB-C18 semipreparative column, 50% Phase A in Phase B, 1 min, and then 50-5% Phase A in Phase B, 10 min)]. MS (ESI-TOF) [m/z (%)]: 590 ([MH]⁺, 100).

DPALM-3. A solution of DPALM-3.1 (30.0 mg, 50.91 μmol) in 500 μL of TFA/CH₂Cl₂/TES (225:225:50) was stirred at rt for 30 min. After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 9.8 mg of DPALM-3.2 as a colorless film [77%, t_{R}=5.7 min (Zorbax SB-C18 semipreparative column, 100% Phase A in Phase B, 4 min, and then 100-95% Phase A in Phase B, 16 min)]. MS (ESI-TOF) [m/z (%)]: 493 ([MH]⁺, 100]. HRMS (ESI-TOF) calculated for [C₂₂H₄₉N₆O₂S₂] ([MH]⁺) 493.3356, found 493.3353. DPALM-3.2 was then dissolved in DMSO containing 100 mM TCEP to yield the free thiol DPALM-3 in quantitative yield.

Synthesis of DPALM-4

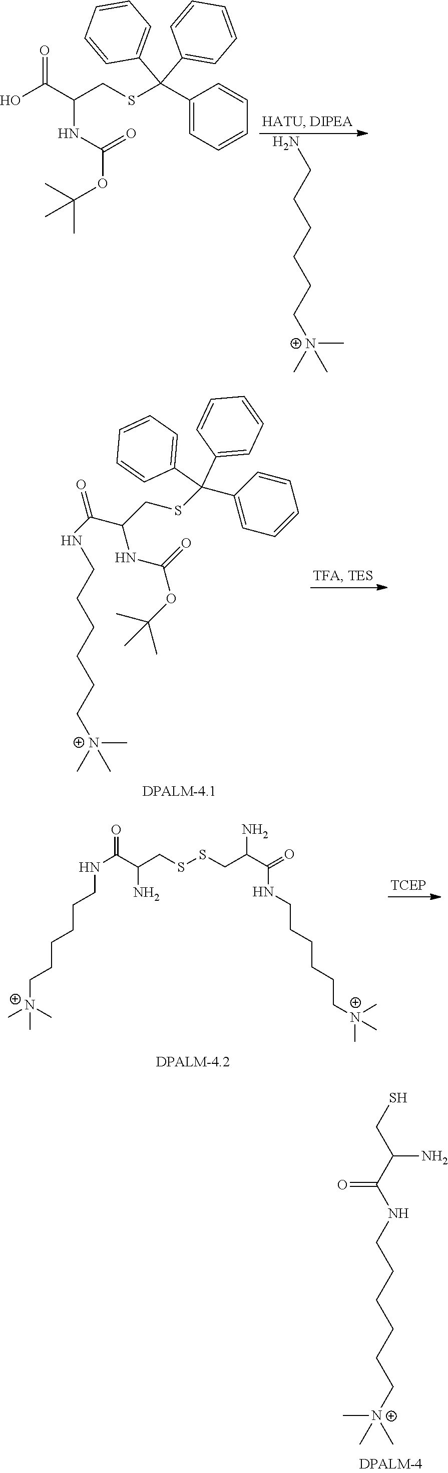

DPALM-4.1. A solution of N-Boc-L-Cys(Trt)-OH (21.7 mg, 46.9 μmol) in CH₂Cl₂ (500 μL) was stirred at 0° C. for 10 min, and then HATU (19.6 mg, 51.5 μmol) and DIEA (32.6 μL, 187.4 μmol) were successively added. After 10 min stirring at 0° C., (6-aminohexyl)trimethylammonium (AHTA) bromide hydrobromide (15.0 mg, 46.9 μmol) was added. After 1 h stirring at rt, the reaction mixture was concentrated, providing a yellow oil. Then, the corresponding residue was diluted in MeOH (250 μL), filtered using a 0.2 μm syringe-driven filter, and the crude solution was purified by HPLC, affording 25.4 mg of DPALM-4.1 as a colorless film [90%, t_{R}=9.8 min (Zorbax SB-C18 semipreparative column, 50% Phase A in Phase B, 1 min, and then 50-5% Phase A in Phase B, 10 min)]. MS (ESI-TOF) [m/z (%)]: 604 ([MH]⁺, 100).

DPALM-4. A solution of DPALM-4.1 (15.0 mg, 24.8 μmol) in 500 μL of TFA/CH₂Cl₂/TES (225:225:50) was stirred at rt for 30 min. After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 5.4 mg of DPALM-4.2 as a colorless film [83%, t_{R}=7.9 min (Zorbax SB-C18 semipreparative column, 100% Phase A in Phase B, 4 min, and then 100-95% Phase A in Phase B, 16 min)]. MS (ESI-TOF) [m/z (%)]: 523 ([MH]⁺, 21], 262 ([MH]²⁺, 100]. DPALM-4.2 was then dissolved in DMSO containing 100 mM TCEP to yield the free thiol DPALM-4 in quantitative yield.

Synthesis of DPALM-5

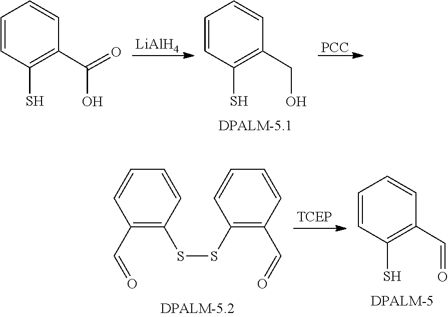

DPALM-5.1. LiAlH₄ (250 mg, 6.59 mmol) was suspended in 5 mL of anhydrous THF. 2-mercaptobenzoid acid (300 mg, 1.95 mmol) in 2 mL of anhydrous THF was added dropwise to the reaction mixture. The reaction was stirred at rt overnight, after which 3 mL EtOAc and 10 mL H₂SO₄ (10%) were added dropwise. The mixture was extracted 2× with EtOAc, the organic fraction washed 1× with brine and then dried with Na₂SO₄. Solvent was removed in vacuo to yield DPALM-5.1 as a yellow oil.

DPALM-5. A flame-dried flask was charged with PCC (342 mg, 1.59 mmol) and dissolved in 10 mL anhydrous CH₂Cl₂. DPALM-5.1 (100 mg, 0.71 mmol) was dissolved in 5 mL anhydrous CH₂Cl₂ and added dropwise to the reaction. The mixture was stirred at rt for 5 h, diluted with 10 mL CH₂Cl₂ and washed 3× with H₂O. The organic fraction was filtered through cotton and then passed through a silica plug with CH₂Cl₂ and dried in vacuo to yield DPALM-5.2 as a white solid. DPALM-5.2 was then dissolved in DMSO containing 100 mM TCEP to yield the free thiol DPALM-5 in quantitative yield.

Synthesis of DPALM-6

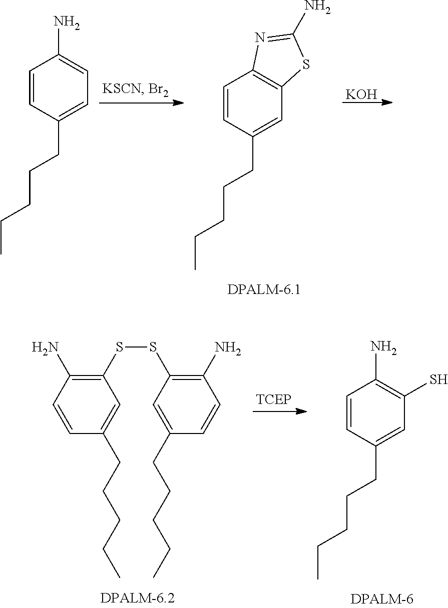

DPALM-6.1. A suspension of 4-pentylaniline (1.00 g, 6.13 mmol) and potassium thiocyanate (2.38 g, 24.50 mmol) in 9 mL AcOH was stirred for 10 min at rt. A solution of bromide (0.98 g, 6.13 mmol) in 4 mL AcOH was slowly added to the reaction over 20 min. The reaction was then stirred at rt for 24 h, after which the mixture was poured into 120 mL cold water made basic with 18 mL NH₄OH (28%). The mixture was extracted 3× with EtOAc, washed 1× with NaCl (Sat.), and dried with Na₂SO₄. Solvent was removed in vacuo and the residue purified by flash chromatography (1:1 EtOAc:Hexanes). Combined product fractions were dried to yield a pale yellow solid.

DPALM-6. DPALM-6.1 (186.4 mg, 0.85 mmol) was refluxed at 120° C. under a flow of N₂ in 20 mL of KOH (10 M) in ethylene glycol for 20 h. The reaction was acidified with HCl (10 M) and extracted 2× with CH₂Cl₂ and washed 1× with NaCl (sat.) before drying with Na₂SO₄. Solvent was removed in vacuo and the residue purified by flash chromatography (2:3 EtOAc:Hexanes). Product fractions were combined and dried to yield DPALM-6.2 as a yellow oil (98.6 mg, 60% yield). DPALM-6.2 was then dissolved in DMSO containing 100 mM TCEP to yield the free thiol DPALM-6 in quantitative yield.

Synthesis of DPALM-7

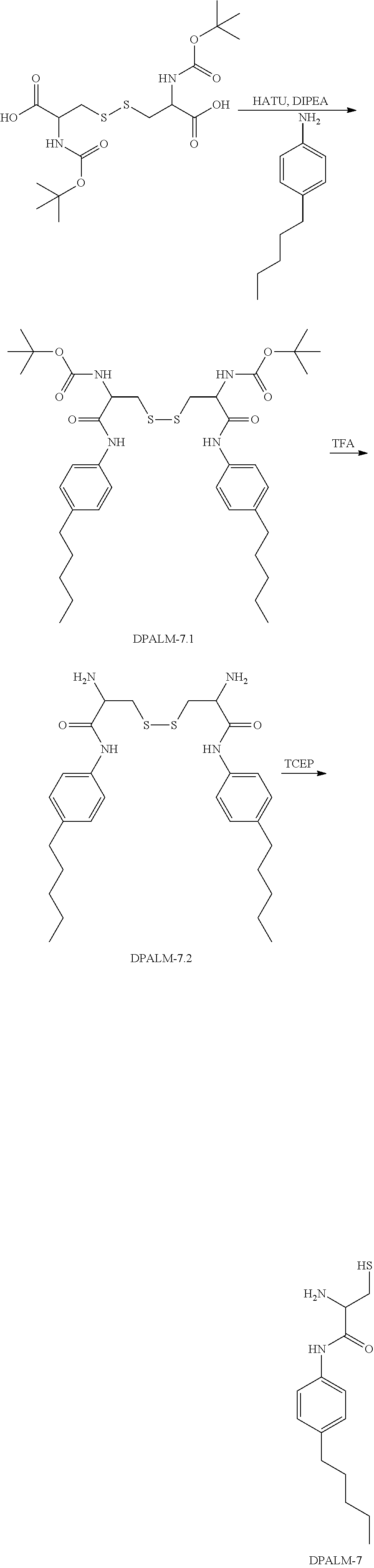

DPALM-7.1. A solution of (Boc-Cys-OH)₂ (100 mg, 227 μmol) in CH₂Cl₂ (5 mL) was stirred at 0° C. for 10 min. HATU (285 mg, 749 μmol) and DIEA (475 μL, 2.72 mmol) were then added and the reaction stirred at 0° C. for 10 min. 4-pentylaniline (121 μL, 681 μmol) was then added and the reaction stirred for 1 h at rt. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography to afford 148 mg of DPALM-7.1 as a white solid (89% yield).

DPALM-7. A solution of DPALM-7.1 (50 mg, 68.4 μmol) in 1 mL of TFA/CH₂Cl₂ (1:1) was stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield 28.3 mg of DPALM-7.2 as a white solid (78% yield). DPALM-7.2 was then dissolved in DMSO containing 100 mM TCEP to yield the free thiol DPALM-7 in quantitative yield.

DPALM-8. BIM-46187 is commercially available.

Synthesis of DPALM-9

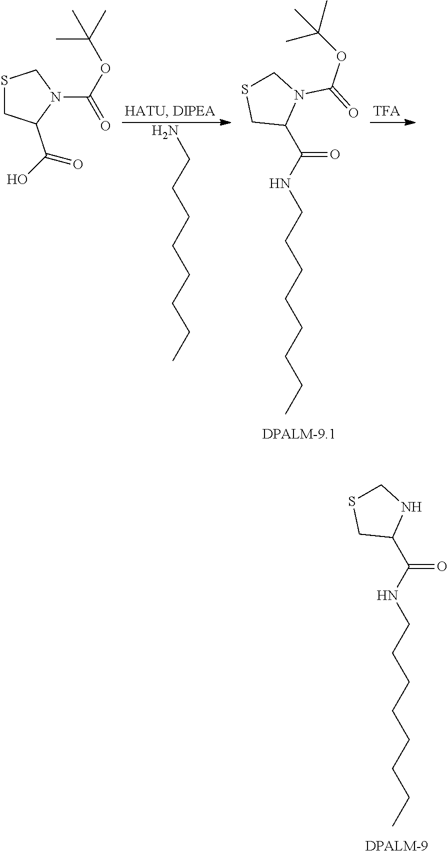

DPALM-9.1. A solution of Boc-Thz (100 mg, 429 μmol) in CH₂Cl₂ (2 mL) was stirred at 0° C. for 10 min. HATU (179 mg, 472 μmol) and DIEA (299 μL, 1.71 mmol) were then added and the reaction stirred at 0° C. for 10 min. octylamine (71 μL, 429 μmol) was then added and the reaction stirred for 1 h at rt. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography (1:1 EtOAc:Hexanes) to afford 126 mg of DPALM-9.1 as a yellow oil (85% yield).

DPALM-9. A solution of DPALM-9.1 (50 mg, 145 μmol) in 1 mL of TFA/CH₂Cl₂ (1:1) was stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield 29.1 mg of DPALM-9 as a white solid (82% yield).

Synthesis of DPALM-10

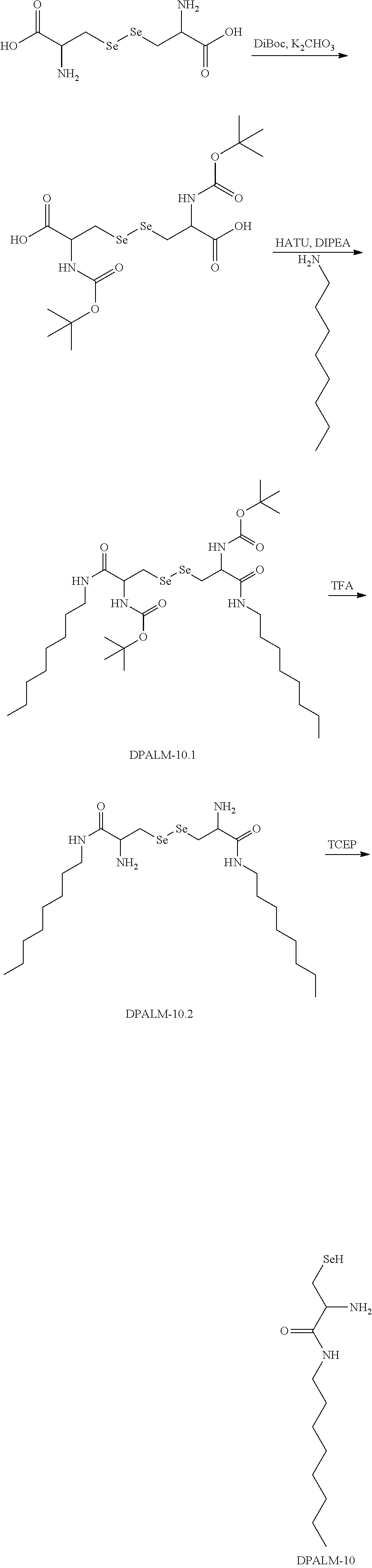

N-Boc-Seleno-L-cysteine. Seleno-L-cysteine (50 mg, 150 μmol) was suspended in 1 mL THF:H₂O (1:1). K₂CO₃ (62.5 mg, 449 μmol) and DiBoc (98 mg, 449 μmol) were added and the reaction stirred vigorously overnight at rt. The reaction mixture was diluted with H₂O and extracted 2× with Et₂O. The aqueous fraction was brought to pH 1-2 by adding 1 M HCl dropwise until the solution became cloudy. The aqueous fraction was then extracted 3× with EtOAc, the organic fraction dried with Na₂SO₄, filtered, and solvent removed in vacuo to yield 72.1 mg of N-Boc-Seleno-L-cysteine as a yellow oil (90% yield).

DPALM-10.1. A solution of N-Boc-Seleno-L-cysteine (72.1 mg, 135 μmol) in CH₂Cl₂ (1.5 mL) was stirred at 0° C. for 10 min. HATU (113 mg, 297 μmol) and DIEA (188 μL, 1.08 mmol) were then added and the reaction stirred at 0° C. for 10 min. Octylamine (38 μL, 270 μmol) was then added and the reaction stirred for 1 h at rt. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography (0-20% EtOAc in Hexanes) to afford 46.1 mg of DPALM-10.1 as a yellow solid (45% yield).

DPALM-10. A solution of DPALM-10.1 (46.1 mg, 61.9 μmol) in 1 mL of TFA/CH₂Cl₂ (1:1) was stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield 27.6 mg of DPALM-10.2 as a yellow solid (81% yield). DPALM-10.2 was then dissolved in DMSO containing 100 mM TCEP to yield the free thiol DPALM-10 in quantitative yield.

Synthesis of DPALM-11

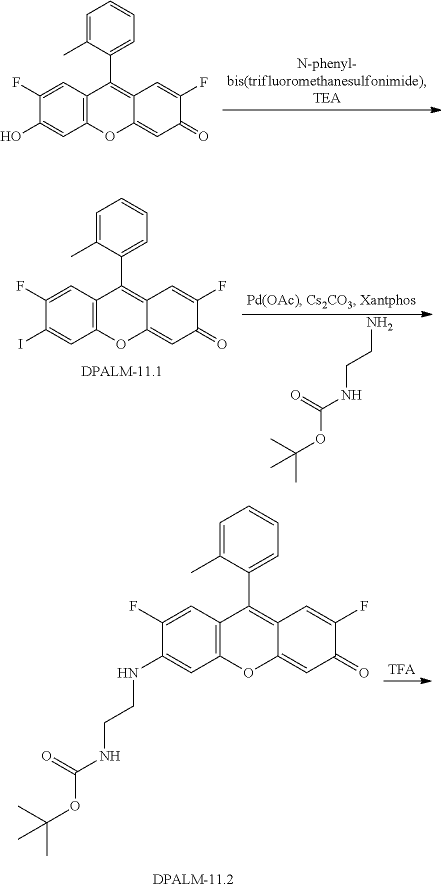 [SMILES: Cc1ccccc1-c1c2cc(F)c(=O)cc-2oc2cc(O)c(F)cc12 ; Cc1ccccc1-c1c2cc(F)c(=O)cc-2oc2cc(I)c(F)cc12 ; CC(C)(C)OC(=O)NCCN ; Cc1ccccc1-c1c2cc(F)c(=O)cc-2oc2cc(NCCNC(=O)OC(C)(C)C)c(F)cc12]

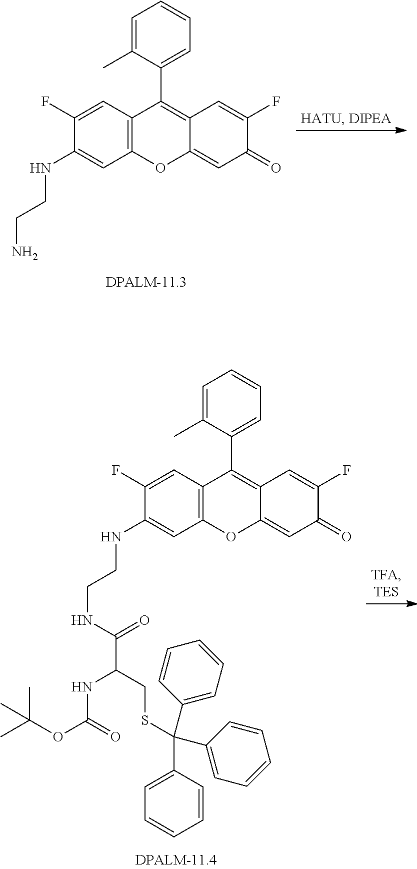 [SMILES: Cc1ccccc1-c1c2cc(F)c(=O)cc-2oc2cc(NCCN)c(F)cc12 ; Cc1ccccc1-c1c2cc(F)c(=O)cc-2oc2cc(NCCNC(=O)C(CSC(c3ccccc3)(c3ccccc3)c3ccccc3)NC(=O)OC(C)(C)C)c(F)cc12]

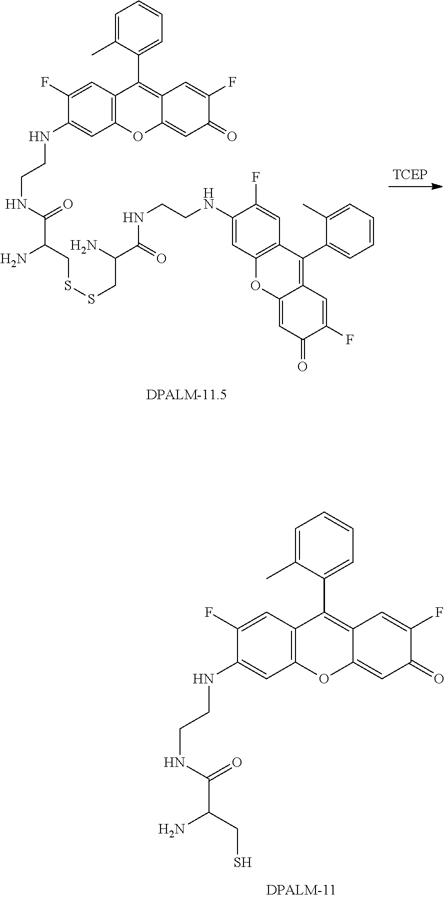 [SMILES: Cc1ccccc1-c1c2cc(F)c(=O)cc-2oc2cc(NCCNC(=O)C(N)CSSCC(N)C(=O)NCCNc3cc4oc5cc(=O)c(F)cc-5c(-c5ccccc5C)c4cc3F)c(F)cc12 ; Cc1ccccc1-c1c2cc(F)c(=O)cc-2oc2cc(NCCNC(=O)C(N)CS)c(F)cc12]

DPALM-11.1. To a dry, argon-flushed round-bottomed flask was added Pennsylvania Green (PennGreen, 50.0 mg, 147.8 μmol) and N-phenyl-bis(trifluoromethanesulfonimide) (63.4 mg, 177.4 μmol). Then, anhydrous 1,4-dioxane (1 mL) was added, followed by Et₃N (24.7 μL, 177.4 μmol). The flask was heated under Ar to 60° C. for 1 h [Note: Conversion to the intermediate triflate was observed by TLC and HPLC-MS]. The flask was removed from the oil bath and LiI (59.3 mg, 443.4 μmol) and a reflux condenser were added. The solution was refluxed (˜110° C.) for 4 h. The flask was cooled to rt. Then, aqueous 2M solution of NaOH (250 L) was added, and the solution was stirred for 1 h. Afterwards, H₂O (3 mL) was added, giving rise to a product slurry that was stirred for 1 h. After this time, the orange slurry was chilled to 4° C., filtered, and the product cake washed with cold H₂O (3×1 mL) to give the product containing residual 1,4-dioxane. This product cake was treated with Et₂O (250 μL), stirred vigorously for 1 min, and hexanes (250 μL) were added. The resulting slurry was filtered and the product cake extensively dried under high vacuum to provide 53.8 mg of DPALM-11.1 as an orange solid [81%]. MS (ESI-TOF) [m/z (%)]: 448 ([MH]⁺, 100).

DPALM-11.2. An oven-dried microwave test tube was charged with DPALM-11.1 (10.0 mg, 22.3 μmol), N-Boc-ethylenediamine (5.3 μL, 33.5 μmol), Pd(OAc)₂ (0.5 mg, 2.2 μmol), Cs₂CO₃ (14.5 mg, 44.6 μmol), and Xantphos (1.9 mg, 3.35 μmol). The test tube was slowly flushed with Ar. Then, anhydrous, degassed toluene (500 μL) was added. The test tube was sealed and heated to 120° C. in a microwave reactor for 1 h. The reaction tube was cooled to rt and diluted with CH₂Cl₂ (2.5 mL). Then, H₂O (1.5 mL) was added, and the organic layer was separated. The aqueous portion was extracted with CH₂Cl₂ (2×1.5 mL). The organic layers were combined, dried over anhydrous Na₂SO₄, and filtered. The solvent was removed under reduced pressure, giving an orange crude solid. The crude was purified by flash chromatography (0-5% MeOH in CH₂Cl₂), affording 6.1 mg of DPALM-11.2 as a red solid [57%, R_{f}=0.16 (2% MeOH in CH₂Cl₂)]. MS (ESI-TOF) [m/z (%)]: 481 ([MH]⁺, 100).

DPALM-11.3. A solution of DPALM-11.2 (3.5 mg, 7.3 μmol) in 500 μL of TFA/CH₂Cl₂ (1:1) was stirred at rt for 15 min. After removal of the solvent, the residue was dried under high vacuum for 3 h and used without further purification. MS (ESI-TOF) [m/z (%)]: 381 ([MH]⁺, 100).

DPALM-11.4. A solution of N-Boc-L-Cys(Trt)-OH (3.4 mg, 7.3 μmol) in CH₂Cl₂ (250 μL) was stirred at 0° C. for 10 min, and then HATU (3.1 mg, 8.0 μmol) and DIEA (5.1 μL, 29.2 μmol) were successively added. After 10 min stirring at 0° C., a DPALM-11.3 (2.77 mg, 7.3 μmol) solution in CH₂Cl₂ (250 μL) containing DIEA (2.5 μL, 14.6 μmol) was added. After 1 h stirring at rt, the reaction mixture was washed with HCl (5%) (3×250 μL) and NaHCO₃(sat) (3×250 μL). The organic layer was dried (Na₂SO₄), filtered and concentrated, providing a red orange solid, which was purified by flash chromatography (0-5% MeOH in CH₂Cl₂), affording 4.9 mg of DPALM-11.4 as a red solid [82%, R_{f}=0.23 (2% MeOH in CH₂Cl₂)]. MS (ESI-TOF) [m/z (%)]: 826 ([MH]⁺, 100).

DPALM-11. A solution of DPALM-11.4 (2.7 mg, 3.3 μmol) in 400 μL of TFA/CH₂Cl₂/TES (180:180:40) was stirred at rt for 30 min. After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 1.5 mg of DPALM-11.5 as a colorless film [92%, t_{R}=6.2 min (Zorbax SB-C18 semipreparative column, 50% Phase A in Phase B, 1 min, then 50-5% Phase A in Phase B, 5 min, and then 5% Phase A in Phase B, 7 min)]. As a disulfide (RS-SR): t_{R}=1.96 min (Eclipse Plus C8 analytical column, 5% Phase A in Phase B, 5.5 min). MS (ESI-TOF) [m/z (%)]: 965 ([MH]⁺, 5), 483 ([MH]²⁺, 100). DPALM-11.5 was then dissolved in DMSO containing 100 mM TCEP to yield the free thiol DPALM-11 in quantitative yield.

Synthesis of DPALM-12

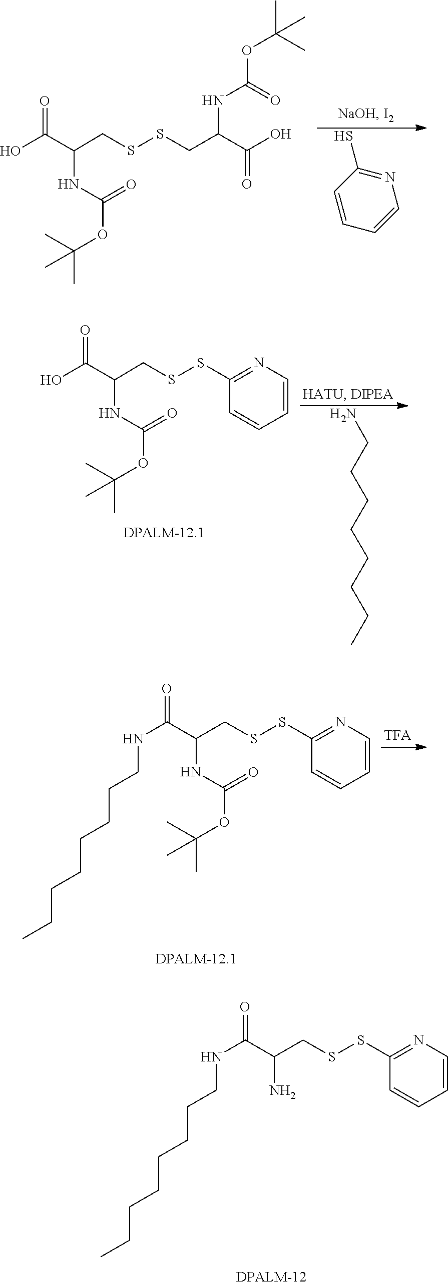

DPALM-12.1. A solution of (Boc-Cys-OH)₂ (250 mg, 568 μmol) in 1:1 THF:H₂O (20 mL) was stirred at 0° C. for 10 min. 2-thiopyridine (757 mg, 6.81 mmol) and 10 M NaOH (795 μL, 7.95 mmol) were added and the reaction mixture stirred for 5 min at 0° C. 12 (25 mg/mL in H₂O) was added dropwise until the reaction turned brown. The reaction was then allowed to react rt with stirring overnight. Solvent was removed in vacuo and the residue taken up in EtOAc and washed 2× with 1 M HCl and 1× with NaCl (sat.). The organic fraction was dried to yield a brown oil which was purified by flash chromatography (20-90% EtOAc in Hexanes+1% AcOH). Product fractions were combined and dried to yield DPALM-12.1 as a yellow solid (227 mg, 61% yield).

DPALM-12. A solution of DPALM 12.1 (20 mg, 60.5 μmol) in 700 μL CH₂Cl₂ was stirred at 0° C. for 10 min. HATU (25.3 mg, 66.6 μmol) and DIPEA (42.2 μL, 242 μmol) were added and the reaction stirred for 10 min at 0° C. Octylamine was added (10 μL, 60.5 μmol) and the reaction stirred at rt for 1 h. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography to afford DPALM-12.2 as a yellow oil. DPALM-12.2 was then dissolved in 1 mL of TFA/CH₂Cl₂ (1:1) and stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield 12.5 mg of DPALM-12 as a yellow solid (60% yield).

Synthesis of DPALM-13

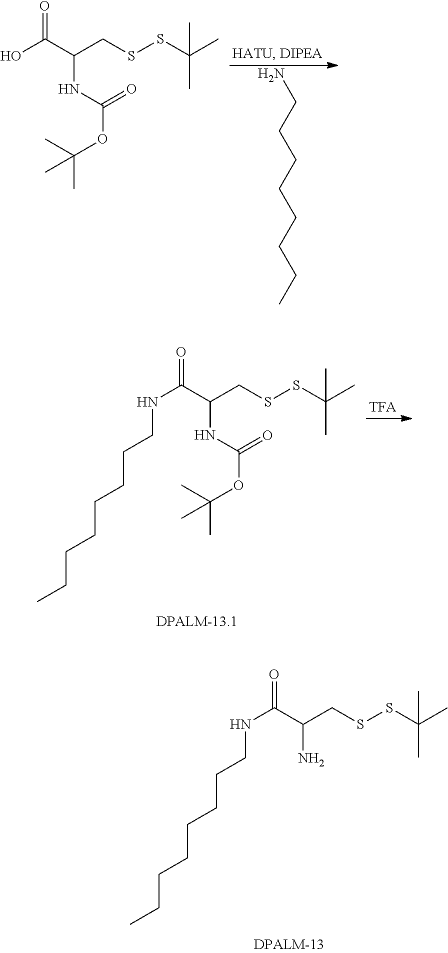

DPALM-13. A solution of S-isopropylthiol-N-Boc-cysteine (200 mg, 646 μmol) in 4 mL CH₂Cl₂ was stirred at 0° C. for 10 min. HATU (270 mg, 711 μmol) and DIPEA (450 μL, 2.59 mmol) were added and the reaction stirred for 10 min at 0° C. Octylamine was added (58.5 μL, 646 μmol) and the reaction stirred at rt for 1 h. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by HPLC to afford DPALM-13.1 as a yellow oil (C18-RP column, 50-95% MeOH in H₂O+0.1% FA). DPALM-13.1 was then dissolved in 1 mL of TFA/CH₂Cl₂ (1:1) and stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield DPALM-13 as a white solid.

Synthesis of DPALM-14

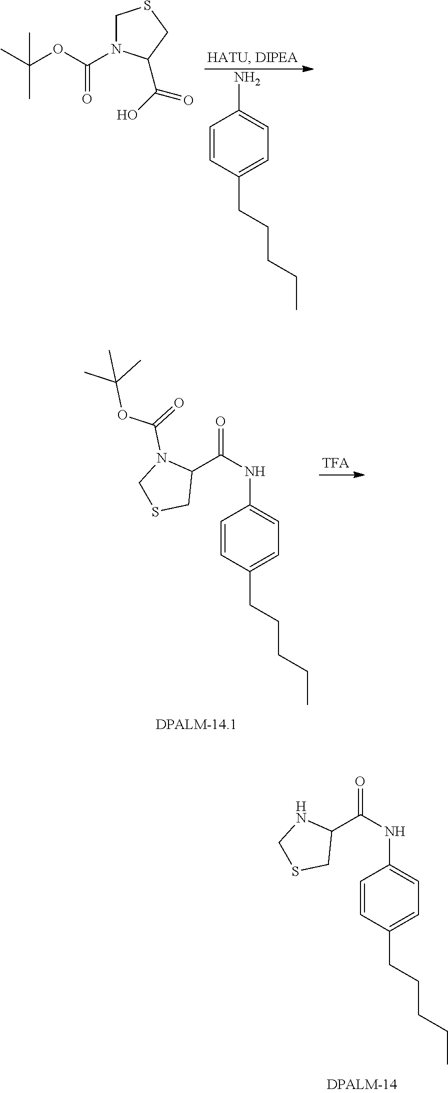

DPALM-14.1. A solution of N-Boc-Thz (250 mg, 1.07 mmol) in 3 mL CH₂Cl₂ was stirred at 0° C. for 10 min. HATU (448 mg, 1.18 mmol) and DIPEA (747 μL, 4.29 mmol) were added and the reaction stirred for 10 min at 0° C. 4-pentylaniline was added (190 μL, 1.07 mmol) and the reaction stirred at rt for 1 h. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by flash chromatography (1:4 EtOAc:Hexanes) to afford DPALM-14.1 as a yellow oil (339 mg, 84% yield).

DPALM-14. DPALM-14.1 (257 mg, 679 μmol) was dissolved in 1 mL of TFA/CH₂Cl₂ (1:1) and stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield DPALM-14 as a white solid (162 mg, 86% yield).

Synthesis of DPALM-15

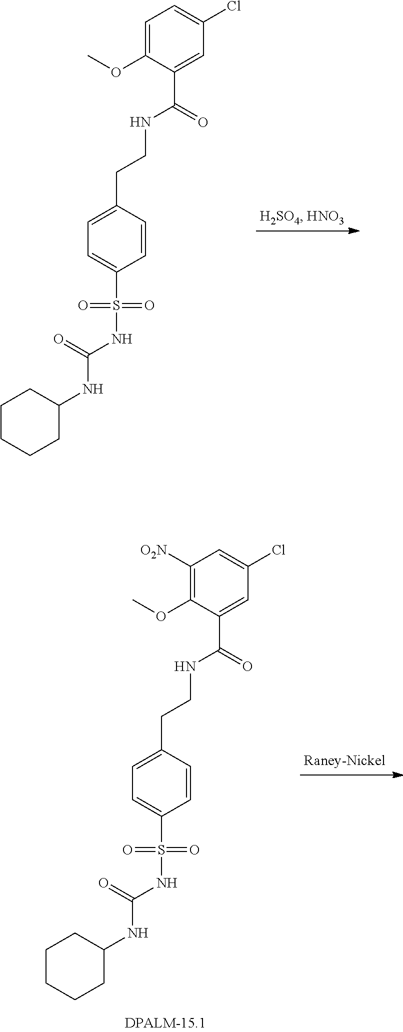 [SMILES: COc1ccc(Cl)cc1C(=O)NCCc1ccc(S(=O)(=O)NC(=O)NC2CCCCC2)cc1 ; COc1c(C(=O)NCCc2ccc(S(=O)(=O)NC(=O)NC3CCCCC3)cc2)cc(Cl)cc1[N+](=O)[O-]]

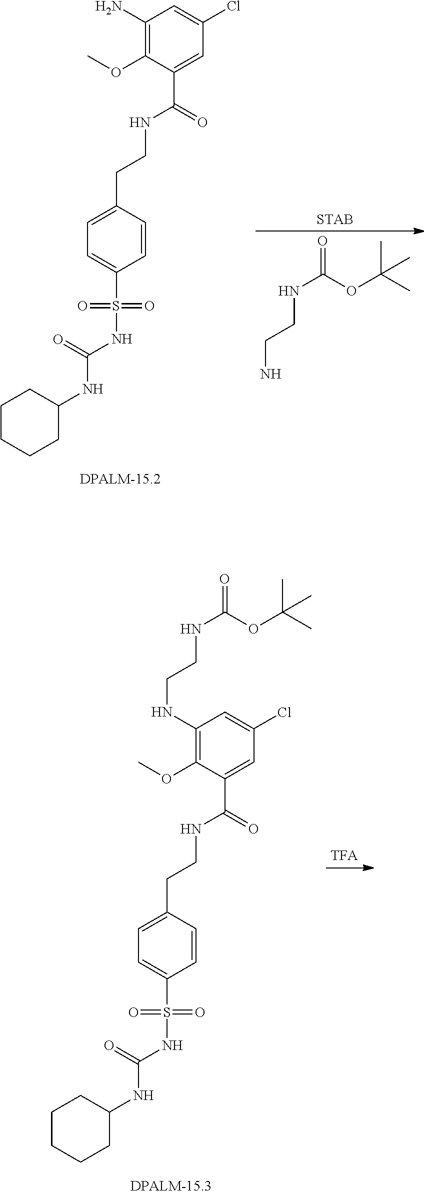 [SMILES: COc1c(N)cc(Cl)cc1C(=O)NCCc1ccc(S(=O)(=O)NC(=O)NC2CCCCC2)cc1 ; CC(C)(C)OC(=O)NCCN ; COc1c(NCCNC(=O)OC(C)(C)C)cc(Cl)cc1C(=O)NCCc1ccc(S(=O)(=O)NC(=O)NC2CCCCC2)cc1]

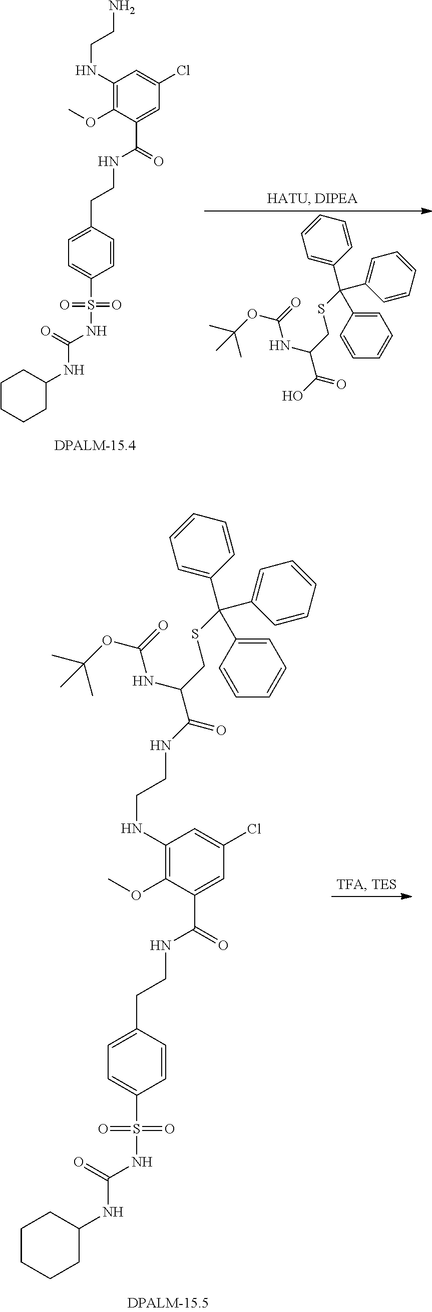 [SMILES: COc1c(NCCN)cc(Cl)cc1C(=O)NCCc1ccc(S(=O)(=O)NC(=O)NC2CCCCC2)cc1 ; CC(C)(C)OC(=O)NC(CSC(c1ccccc1)(c1ccccc1)c1ccccc1)C(=O)O ; COc1c(NCCNC(=O)C(CSC(c2ccccc2)(c2ccccc2)c2ccccc2)NC(=O)OC(C)(C)C)cc(Cl)cc1C(=O)NCCc1ccc(S(=O)(=O)NC(=O)NC2CCCCC2)cc1]

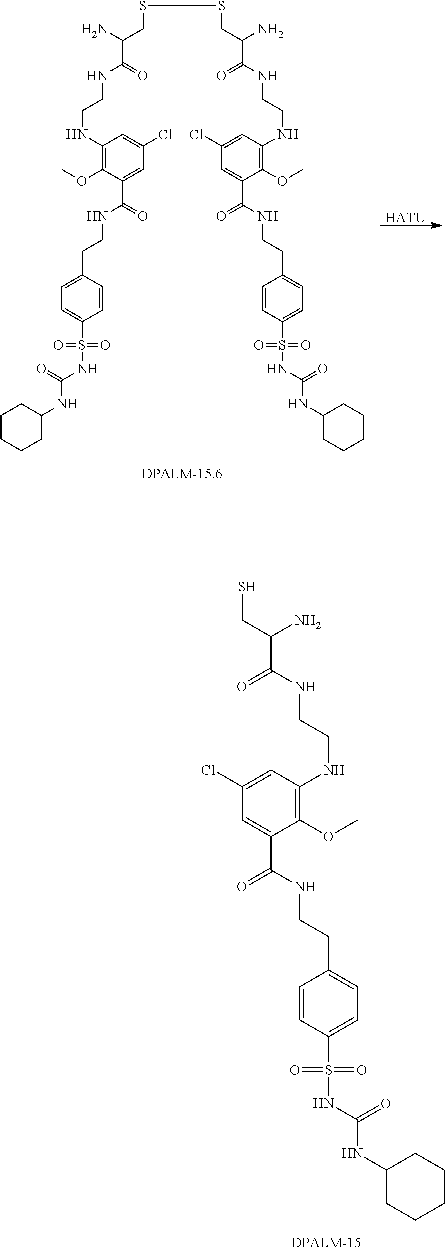 [SMILES: COc1c(NCCNC(=O)C(N)CSSCC(N)C(=O)NCCNc2cc(Cl)cc(C(=O)NCCc3ccc(S(=O)(=O)NC(=O)NC4CCCCC4)cc3)c2OC)cc(Cl)cc1C(=O)NCCc1ccc(S(=O)(=O)NC(=O)NC2CCCCC2)cc1 ; "HATU" ; COc1c(NCCNC(=O)C(N)CS)cc(Cl)cc1C(=O)NCCc1ccc(S(=O)(=O)NC(=O)NC2CCCCC2)cc1]

DPALM-15.1. Concentrated H₂SO₄ (10 mL) was added to concentrated HNO₃ (10 mL) in an ice bath. To the mixture, Glibenclamide (3.0 g, 6.1 mmol) as a suspension in 1,4-dioxane (4 mL) was slowly added. After 30 min stirring at 0° C., the mixture was warmed to rt and stirred for 1 h [Note: Keep the reaction scale and temperature. If not, the overheated reaction mixture will boil vigorously]. The reaction mixture was partitioned between H₂O (20 mL) and EtOAc (20 mL). The organic layer was washed with H₂O (2×10 mL), NaHCO₃ (sat.) (2×10 mL), NaCl (sat.) (2×10 mL), dried over Na₂SO₄ and concentrated in vacuo. The reaction crude was dissolved in hot EtOH (100 mL) and slowly cooled. The sonication of the mixture resulted in an emulsion-like precipitate. The resulting precipitate was collected by filtration to yield 352.9 mg of DPALM-15.1 as a white solid [11%]. MS (ESI-TOF) [m/z (%)]: 539 ([MH]⁺, 100).

DPALM-15.2. Raney-Nickel (2800, slurry, in H₂O, active catalyst) (150 μL) was added to DPALM-15.1 (185.0 mg, 343.8 μmol) in anhydrous THF (5 mL). Then, the mixture was hydrogenated at balloon pressure for 8 h. The resulting suspension was filtered through Celite, rinsed with THF (3×1 mL) and concentrated, obtaining 170.3 mg of DPALM-15.2 as a pale yellow foam [97%] [Note: The product was used in the next step without further purification]. MS (ESI-TOF) [m/z (%)]: 509 ([MH]⁺, 100).

DPALM-15.3. A solution of DPALM-15.2 (25.0 mg, 49.2 μmol) and N-Boc-2-aminoacetaldehyde (9.4 mg, 59.0 μmol) in DMF (250 μL) was stirred at rt for 30 min. Then, sodium triacetoxyborohydride (STAB, 52.1 mg, 246.0 μmol) was added, and the mixture was stirred at rt for 12 h. After removal of the solvent, the residue was diluted in MeOH (500 μL), filtered using a 0.2 μm syringe-driven filter, and the crude solution was purified by HPLC, affording 25.9 mg of DPALM-15.3 as a white solid [81%, t_{R}=8.6 min (Zorbax SB-C18 semipreparative column, 50% Phase A in Phase B, 1 min, then 50-5% Phase A in Phase B, 5 min, and then 5% Phase A in Phase B, 7 min)]. MS (ESI-TOF) [m/z (%)]: 674 ([M+Na]⁺, 12), 652 ([MH]⁺, 100).

DPALM-15.4. A solution of DPALM-15.3 (4.0 mg, 6.1 μmol) in 500 μL of TFA/CH₂Cl₂ (1:1) was stirred at rt for 15 min. After removal of the solvent, the residue was dried under high vacuum for 3 h and used without further purification. MS (ESI-TOF) [m/z (%)]: 552 ([MH]⁺, 100).

DPALM-15.5. A solution of N-Boc-L-Cys(Trt)-OH (2.9 mg, 6.1 μmol) in DMF (150 μL) was stirred at 0° C. for 10 min, and then HATU (2.6 mg, 6.7 μmol) and DIEA (4.3 μL, 24.6 μmol) were successively added. After 10 min stirring at 0° C., a DPALM-15.4 (3.4 mg, 6.1 μmol) solution in DMF (150 μL) containing DIEA (2.1 μL, 12.3 μmol) was added. After 1 h stirring at rt, the reaction mixture was concentrated under reduced pressure, giving a pale yellow solid. Then, the corresponding residue was diluted in MeOH (250 μL), filtered using a 0.2 μm syringe-driven filter, and the crude solution was purified by HPLC, affording 5.6 mg of DPALM-15.5 as a white solid [92%, t_{R}=9.8 min (Zorbax SB-C18 semipreparative column, 50% Phase A in Phase B, 1 min, then 50-5% Phase A in Phase B, 5 min, and then 5% Phase A in Phase B, 7 min)]. MS (ESI-TOF) [m/z (%)]: 997 ([MH]⁺, 100).

DPALM-15. A solution of DPALM-15.5 (4.0 mg, 4.0 μmol) in 400 μL of TFA/CH₂Cl₂/TES (180:180:40) was stirred at rt for 30 min. After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 2.2 mg of DPALM-15.6 as a white solid [84%, t_{R}=5.8 min (Zorbax SB-C18 semipreparative column, 50% Phase A in Phase B, 5 min, and then 5% Phase A in Phase B, 10 min)]. MS (ESI-TOF) [m/z (%)]: 1307 ([MH]⁺, 6), 654 ([MH]⁺², 100). DPALM-15.6 was then dissolved in DMSO containing 100 mM TCEP to yield the free thiol DPALM-15 in quantitative yield.

Synthesis of DPALM-16

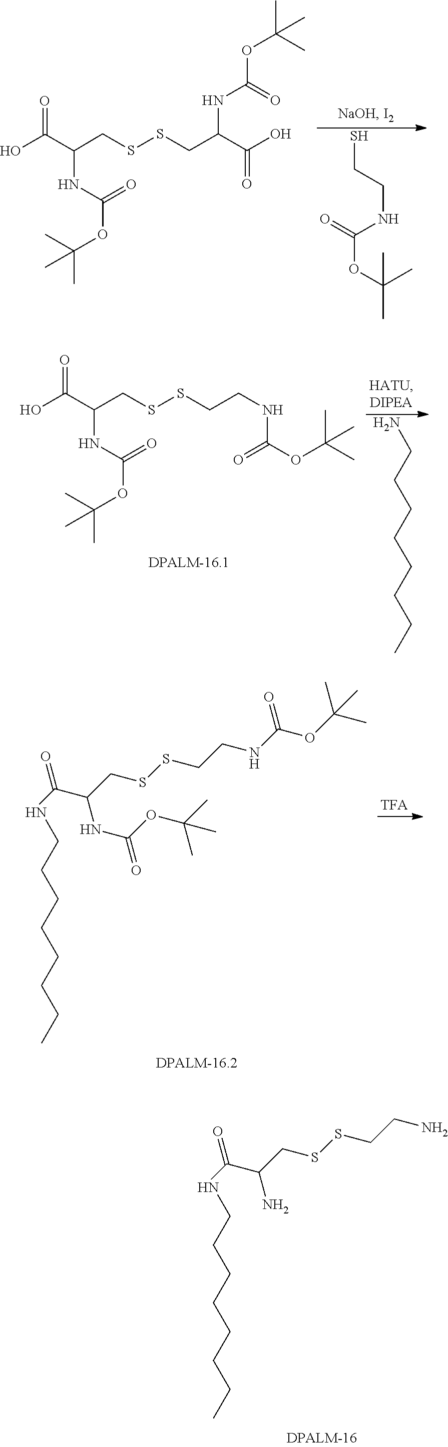

DPALM-16.1. A solution of (Boc-Cys-OH)₂ (50 mg, 113.5 μmol) in 1:1 THF:H₂O (4 mL) was stirred at 0° C. for 10 min. N-Boc-cystamine (241.4 mg, 1.36 mmol) and 10 M NaOH (84.1 μL, 1.59 mmol) were added and the reaction mixture stirred for 5 min at 0° C. I₂ (25 mg/mL in H₂O) was added dropwise until the reaction turned brown. The reaction was then allowed to react rt with stirring overnight. Solvent was removed in vacuo and the residue taken up in EtOAc and washed 2× with 1 M HCl and 1× with NaCl (sat.). The organic fraction was dried to yield a brown oil which was purified by flash chromatography (20-90% EtOAc in Hexanes+1% AcOH). Product fractions were combined and dried to yield DPALM-16.1 as a yellow solid (58.4 mg, 65% yield).

DPALM-16. A solution of DPALM 16.1 (25 mg, 63.4 μmol) in 700 μL CH₂Cl₂ was stirred at 0° C. for 10 min. HATU (26.5 mg, 69.7 μmol) and DIPEA (44.2 μL, 253.5 μmol) were added and the reaction stirred for 10 min at 0° C. Octylamine was added (5.7 μL, 63.4 μmol) and the reaction stirred at rt for 1 h. The reaction mixture was then diluted in CH₂Cl₂ and washed 2× with 1 M HCl and 1× with saturated NaHCO₃. The organic fraction was dried with Na₂SO₄, filtered, and solvent removed in vacuo to yield a yellow oil, which was purified by HPLC (C18-RP column 50-95% MeOH in H₂O+0.1% FA) to afford DPALM-16.2 as a white solid. DPALM-16.2 was then dissolved in 1 mL of TFA/CH₂Cl₂ (1:1) and stirred at rt for 30 min. After removal of the solvent, the residue was taken up in CH₂Cl₂ and washed 2× with 1 M NaOH. The aqueous fractions were then washed 1× with CH₂Cl₂. The organic fractions were combined and dried with Na₂SO₄, filtered, and solvent removed in vacuo to yield of DPALM-16 as a white solid.

1. A compound, or a pharmaceutically acceptable salt thereof, having the formula: 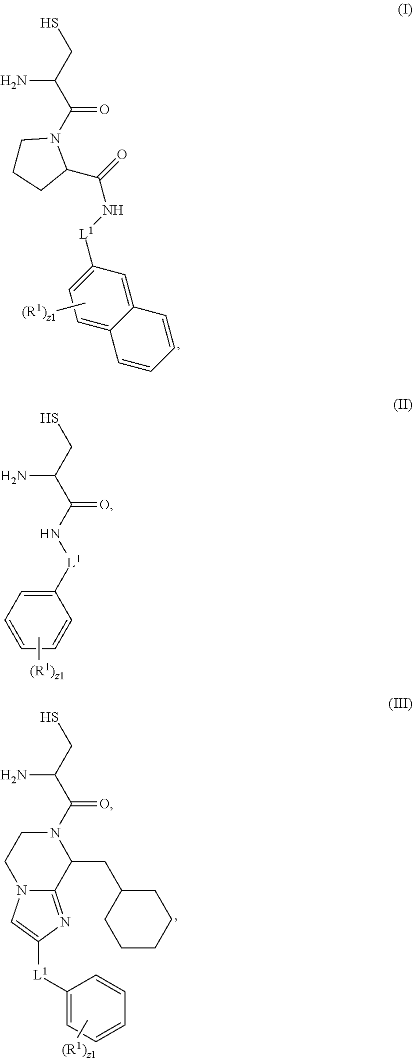 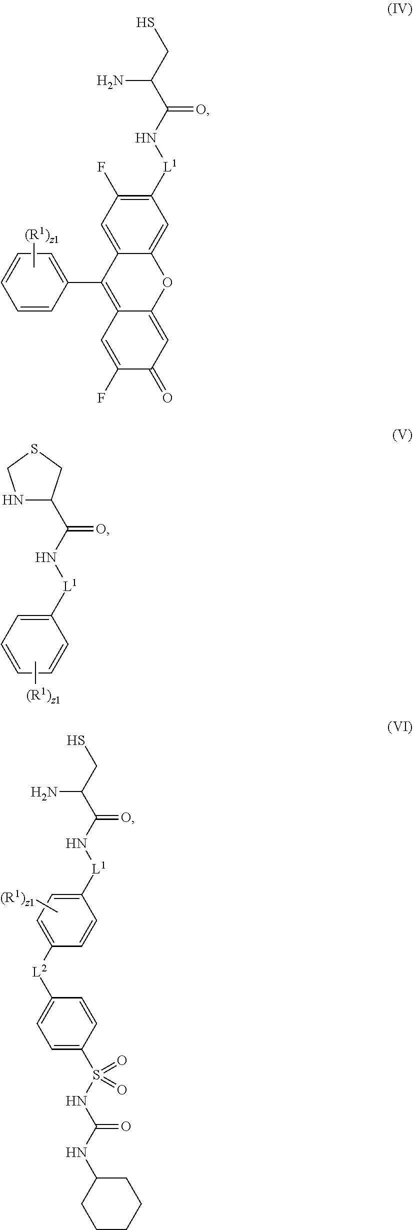 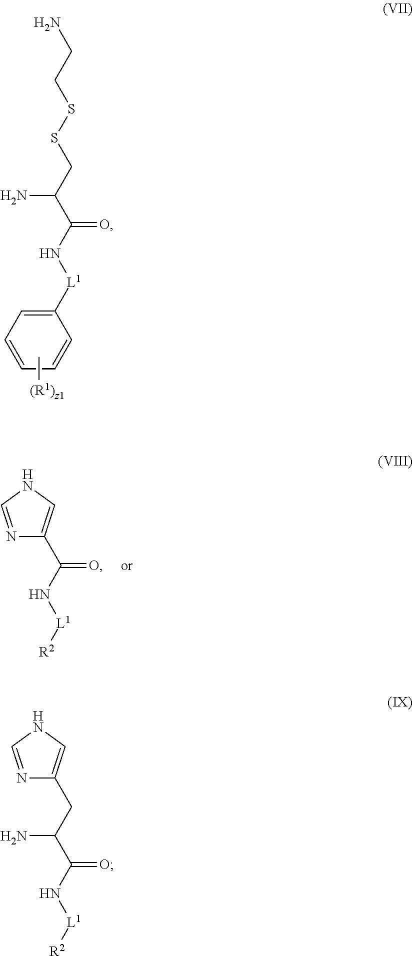
wherein
L¹ is a bond, substituted or unsubstituted C₁-C₁₀ alkylene, or substituted or unsubstituted 2 to 10 membered heteroalkylene;
L² is a bond, substituted or unsubstituted C₁-C₁₀ alkylene, or substituted or unsubstituted 2 to 10 membered heteroalkylene;
R¹ is independently a halogen, substituted or unsubstituted C₁-C₂₀ alkyl, or substituted or unsubstituted 2 to 20 membered heteroalkyl;
R² is hydrogen, halogen, substituted or unsubstituted C₁-C₂₀ alkyl, or substituted or unsubstituted 2 to 20 membered heteroalkyl; and
z1 is an integer from 0 to 7.

2. The compound of claim 1, wherein L¹ is a bond, unsubstituted C₁-C₁₀ alkylene, or unsubstituted 2 to 10 membered heteroalkylene.

3. The compound of claim 1, wherein L¹ is a bond.

4. (canceled)

5. The compound of claim 1, wherein L¹ is —CH₂—CH₂—NH—.

6. The compound of claim 1, wherein L² is a bond or oxo-substituted 2 to 10 membered heteroalkylene.

7. The compound of claim 1, wherein L² is —C(O)NH-(unsubstituted C₁-C₆ alkylene)-.

8. The compound of claim 1, wherein L² is —C(O)NH—CH₂—CH₂—.

9. The compound of claim 1, wherein R¹ is independently a halogen, unsubstituted C₁-C₁₀ alkyl, or substituted or unsubstituted 2 to 10 membered heteroalkyl.

10. The compound of claim 1, wherein R¹ is independently a halogen, unsubstituted C₁-C₆ alkyl, or unsubstituted C₁-C₆ alkoxy.

11. The compound of claim 1, wherein R¹ is independently —Cl.

12. (canceled)

13. (canceled)

14. The compound of claim 1, wherein z1 is 0, 1, 2, or 3.

15. (canceled)

16. The compound of claim 1, wherein R² is an unsubstituted C₁-C₂₀ alkyl, or unsubstituted 2 to 20 membered heteroalkyl.

17. (canceled)

18. The compound of claim 1, having the formula:

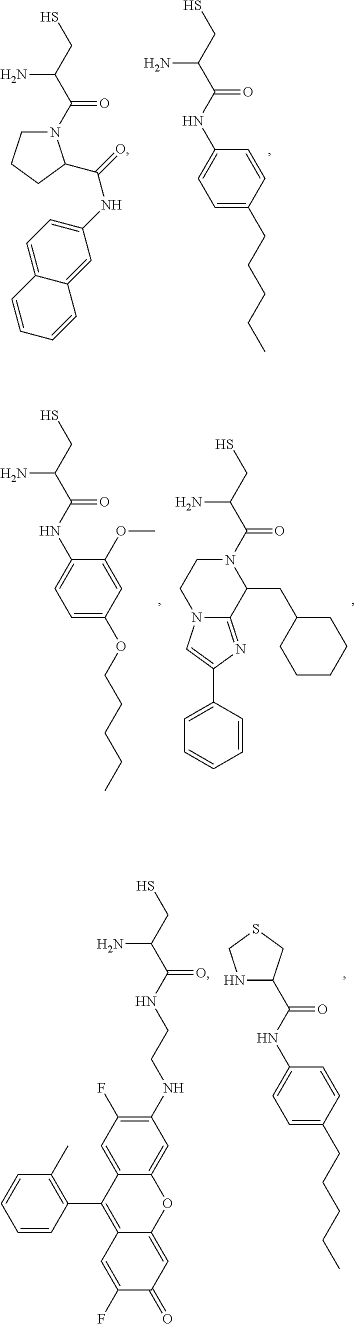

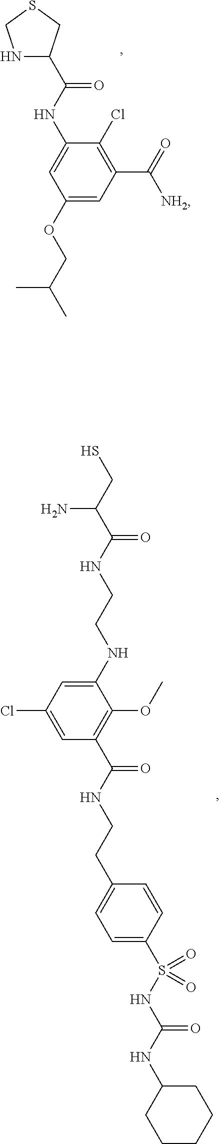 [SMILES: CC(C)COc1cc(NC(=O)C2CSCN2)c(Cl)c(C(N)=O)c1 ; COc1c(NCCNC(=O)C(N)CS)cc(Cl)cc1C(=O)NCCc1ccc(S(=O)(=O)NC(=O)NC2CCCCC2)cc1]

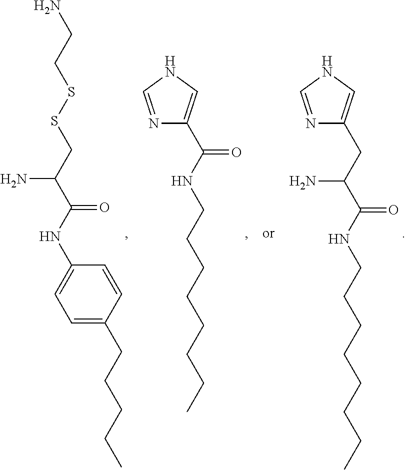 [SMILES: CCCCCc1ccc(NC(=O)C(N)CSSCCN)cc1 ; CCCCCCCCNC(=O)c1c[nH]cn1 ; CCCCCCCCNC(=O)C(N)Cc1c[nH]cn1]

19. A pharmaceutical composition comprising a compound of claim 1, or pharmaceutically acceptable salt thereof, and a pharmaceutically acceptable excipient.

20. A method of treating a depalmitoylation-associated disease in a subject in need thereof, said method comprising administering to the subject a therapeutically effective amount of a compound of claim 1.

21. The method of claim 20, wherein the depalmitoylation-associated disease is a cancer or a neurodegenerative disease.

22. The method of claim 20, wherein the depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis, or glioma.

23. A method of depalmitoylating a protein in a cell, said method comprising contacting the cell with an effective amount of a compound of claim 1.

24. (canceled)

25. The method of claim 23, wherein the protein is HRas, NRas, EGFR, amyloid precursor protein (APP), BACE1, EZH2, PD-L1, flotillin-1, flotillin-2, calnexin, Gα(i), metadherin, CD44, or SNAP25.

26. (canceled)

27. (canceled)

28. The method of claim 23, wherein the cell forms part of a mammalian subject, and wherein said mammalian subject suffers from a cancer or a neurodegenerative disease.

29. (canceled)
